(19) United States
(12) Patent Application Publication
(10) Pub. No.: US 2018/0311347 A1
(43) Pub. Date: Nov. 1, 2018

(54) POLYMORPHISMS IN CACHEXIA PREDICT CLINICAL OUTCOMES OF COLORECTAL CANCER PATIENTS RECEIVING IRINOTECAN AND BEVACIZUMAB

(71) Applicant: University of Southern California, Los Angeles, CA (US)

(72) Inventor: Heinz-Josef Lenz, Los Angeles, CA (US)

(21) Appl. No.: 15/962,185

(22) Filed: Apr. 25, 2018

Related U.S. Application Data
(60) Provisional application No. 62/489,906, filed on Apr. 25, 2017.

(51) Int. Cl. A61K 39/395; A61K 31/4745; C07K 16/22; A61K 45/06; A61P 35/00

(52) U.S. Cl. CPC A61K 39/3955; A61K 31/4745; A61K 2039/505; A61K 45/06; A61P 35/00; C07K 16/22

(57) ABSTRACT

Methods are provided for treating colorectal cancer patients with a therapy comprising irinotecan and bevacizumab. The methods entail administering the therapy to the patient if the patient has specific rs1792689, rs2268753, rs17776182, rs7570532 and/or rs4946935 polymorphisms.

### CROSS-REFERENCE TO RELATED APPLICATION

This application claims the benefit under 35 U.S.C. § 119(e) of U.S. Provisional application No. 62/489,906, filed Apr. 25, 2017, the contents of which are incorporated by reference in its entirety.

### BACKGROUND

In nature, organisms of the same species usually differ from each other in some aspects, e.g., their appearance. The differences are genetically determined and are referred to as polymorphism. Genetic polymorphism is the occurrence in a population of two or more genetically determined alternative phenotypes due to different alleles. Polymorphism can be observed at the level of the whole individual (phenotype), in variant forms of proteins and blood group substances (biochemical polymorphism), morphological features of chromosomes (chromosomal polymorphism) or at the level of DNA in differences of nucleotides (DNA polymorphism).

Polymorphism also plays a role in determining differences in an individual's response to drugs. Pharmacogenetics and pharmacogenomics are multidisciplinary research efforts to study the relationship between genotype, gene expression profiles, and phenotype, as expressed in variability between individuals in response to or toxicity from drugs. Indeed, it is now known that cancer chemotherapy is limited by the predisposition of specific populations to drug toxicity or poor drug response.

Although considerable research correlating gene expression and/or polymorphisms has been reported, much work remains to be done. This disclosure supplements the existing body of knowledge.

### SUMMARY

The Activin/TGFbeta/SMAD pathway, known as cachexia pathway, plays a critical role in development and progression of colorectal cancer. As described herein, single nucleotide polymorphisms (SNPs) of genes involved in the cachexia pathway predict clinical outcomes in irinotecan and bevacizumab (BV)-treated metastatic colorectal cancer (mCRC) patients.

It is described herein that colorectal cancer patients harboring certain genotypes are likely to experience more desirable clinical outcomes when treated with a therapy comprising, or consisting essentially of, or yet further consisting of, irinotecan and bevacizumab, as compared to those not having the genotype. More desirable clinical outcomes for a cancer patient following a therapy include, without limitation, higher likelihood to respond to the therapy, relatively longer progression free survival (PFS), relatively longer overall survival (OS), relatively longer time to tumor recurrence (TTR), lower likelihood to experience an adverse effect or toxicity, or relatively milder adverse effect or toxicity.

The findings of the present disclosure are summarized in the following table.

| | Nearby | Clinical | Favorable | Unfavorable |
|---|---|---|---|---|
| Polymorphism | Gene | Endpoint | Genotype | Genotype |
| rs1792689 | SMAD2 | PFS, OS | G/G | A/G or A/A |
| rs2268753 | ACVR2B | PFS, OS, TR | C/T or C/C | T/T |
| rs17776182 | INHBA | PFS, OS | G/G | A/G or A/A |
| rs7570532 | MSTN | OS | A/A | A/G |
| rs4946935 | FOXO3 | OS | A/G or G/G | A/A |

* The genotypes noted here only refer to one DNA strand; for instance, genotype C/G is equivalent to G/C on the opposite strand and should be understood to encompass both strands.

The rs1792689 polymorphism is located at chromosome position 26931010 on chromosome 18 according to the Genome Reference Consortium Human Build 38 patch release 2 (GRCh38.p2, NCBI). The rs1792689 polymorphism is located within the SMAD family member 2 (SMAD2; Mothers against decapentaplegic homolog 2) gene (in reverse orientation of contig). SMAD2 mediates the signal of the transforming growth factor (TGF)-beta, and thus regulates multiple cellular processes, such as cell proliferation, apoptosis, and differentiation. This protein is recruited to the TGF-beta receptors through its interaction with the SMAD anchor for receptor activation (SARA) protein. In response to TGF-beta signal, this protein is phosphorylated by the TGF-beta receptors. The following nucleotide sequence represents a region of human DNA comprising, or consisting essentially of, or yet further consisting of the rs1792689 polymorphism (in reverse orientation of SMAD2 gene):

(SEQ ID NO: 1)
TATCTACATTCTCTCTCAGGTGTTC[C/T]ATTTTGGATGATGGTGAATA
ATAAG

The rs2268753 polymorphism is located at chromosome position 38448698 on chromosome 3 according to the Genome Reference Consortium Human Build 38 patch release 2 (GRCh38.p2, NCBI). The rs2268753 polymorphism is located within the activin A receptor, type IIB (ACVR2B) gene. ACVR2B is a heteromeric transmembrane receptor with serine/threonine kinase activity that binds to activins, which are dimeric growth and differentiation factors belonging to the transforming growth factor-beta (TGF-beta) superfamily. The following nucleotide sequence represents a region of human DNA comprising, or consisting essentially of, or yet further consisting of the rs2268753 polymorphism:

(SEQ ID NO: 2)
GTATCGGTTCAGGAGTTTAGATCCA[C/T]TCACGGATACTGACCTGTCA
CCATG

The rs17776182 polymorphism is located at chromosome position 41708280 on chromosome 7 according to the Genome Reference Consortium Human Build 38 patch release 2 (GRCh38.p2, NCBI). The rs17776182 polymorphism is located within the INHBA antisense RNA 1 (INHBA-AS1) gene. INHBA is a subunit of both activin and inhibin. The following nucleotide sequence represents a region of human DNA comprising, or consisting essentially of, or yet further consisting of the rs17776182 polymorphism:

(SEQ ID NO: 3)
TGTTTTTAGATGAAGGTGGAAATAC[A/G]ATGAAGATGATGCTCTGTTA
GTTAT

The rs7570532 polymorphism is located at chromosome position 190058686 on chromosome 2 according to the Genome Reference Consortium Human Build 38 patch release 2 (GRCh38.p2, NCBI). The rs7570532 polymorphism is located within the MSTN gene. MSTN or myostatin (also known as growth differentiation factor 8, abbreviated GDF-8) is a myokine, a protein produced and released by myocytes that acts on muscle cells' autocrine function to inhibit myogenesis: muscle cell growth and differentiation. In humans it is encoded by the MSTN gene. Myostatin is a secreted growth differentiation factor that is a member of the TGF beta protein family. The following nucleotide sequence represents a region of human DNA comprising, or consisting essentially of, or yet further consisting of the rs7570532 polymorphism:

(SEQ ID NO: 17)
ATACTATTTAACCATAAAAAAGAGT[A/G]AAGGAATGTC
TTTTGCAGCAAATTA

The rs4946935 polymorphism is located at chromosome position 108679539 on chromosome 6 according to the Genome Reference Consortium Human Build 38 patch release 7 (GRCh38.p7, NCBI). The rs4946935 polymorphism is located within the FOXO3 gene. FOXO3 belongs to the 0 subclass of the forkhead family of transcription factors which are characterized by a distinct fork head DNA-binding domain. There are three other FOXO family members in humans, FOXO1, FOXO4 and FOXO6. These transcription factors share the ability to be inhibited and translocated out of the nucleus on phosphorylation by proteins such as Akt/PKB in the PI3K signaling pathway (aside from FOXO6, which may be constitutively nuclear). Other post-translational modifications including acetylation and methylation are seen and can result in increased or altered FOXO3a activity. This protein likely functions as a trigger for apoptosis through upregulation of genes necessary for cell death, or downregulation of anti-apoptotic proteins such as FLIP. The following nucleotide sequence represents a region of human DNA comprising, or consisting essentially of, or yet further consisting of the rs4946935 polymorphism: AAGGACCCACCAAAA CACCCCTAAT[A/G]TGGCTTTCTT TATCTCCCAA (SEQ ID NO:18)

In one aspect provided is a method for treating a colorectal cancer patient with a therapy comprising, or consisting essentially of, or yet further consisting of an effective amount of irinotecan and bevacizumab, wherein a sample isolated from the patient is characterized by a polymorphism of the group of (G/G) for rs1792689, (C/T) or (C/C) for rs2268753, (G/G) for rs17776182, (A/A) for rs7570532, and (A/G) or (G/G) for rs4946935.

In another aspect provided is a method for treating a colorectal cancer patient with a therapy excluding an effective amount of irinotecan and bevacizumab, wherein a sample isolated from the patient is characterized by a polymorphism of the group of (A/G) or (A/A) for rs1792689, (T/T) for rs2268753, (A/G) or (A/A) for rs17776182, (A/G) for rs7570532, and (A/A) for rs4946935.

In another aspect provided is a method for treating a colorectal cancer patient with an effective amount of a therapy comprising, or consisting essentially of, or yet further consisting of irinotecan and bevacizumab, the method comprising, or consisting essentially of, or yet further consisting of determining if the patient's sample comprises a polymorphism from the group of rs1792689, rs2268753, rs17776182, rs7570532 and rs4946935; and if the patient has (G/G) for rs1792689, (C/T) or (C/C) for rs2268753, (G/G) for rs17776182, (A/A) for rs7570532, and (A/G) or (G/G) for rs4946935, then administering an effective amount of the therapy.

In some embodiments, provided are methods for selecting a colorectal cancer patient for a therapy comprising, or alternatively consisting essentially of, or consisting of, irinotecan and bevacizumab, comprising, or alternatively consisting essentially of, or consisting of, screening a biological sample isolated from the patient for an rs1792689, rs2268753, and/or rs17776182 polymorphism, and selecting the patient for the therapy if the genotype of (G/G) for rs1792689, (C/T) or (C/C) for rs2268753, or (G/G) for rs17776182 is present in the sample. In some embodiments, the patient is not selected for a therapy comprising, or alternatively consisting essentially of, or consisting of, a therapeutically effective amount of irinotecan and bevacizumab if the genotype of (G/G) for rs1792689, (C/T) or (C/C) for rs2268753, or (G/G) for rs17776182 is not present in the sample. In some embodiments, the patient is not selected for a therapy comprising, or alternatively consisting essentially of, or consisting of, irinotecan and bevacizumab if the genotype of (A/G) or (A/A) for rs1792689, (T/T) for rs2268753, or (A/G) or (A/A) for rs17776182 is present in the sample. In some embodiments, the patient is selected for an irinotecan-free and/or oxaliplatin-free therapy if the genotype of (G/G) for rs1792689, (C/T) or (C/C) for rs2268753, or (G/G) for rs17776182 is not present in the sample. In some embodiments, the patient is selected for an irinotecan-free and/or bevacizumab-free therapy if the genotype of (A/G) or (A/A) for rs1792689, (T/T) for rs2268753, or (A/G) or (A/A) for rs17776182 is present in the sample.

Also provided, in some embodiments, are methods for classifying a colorectal cancer patient as eligible for a therapy comprising, or alternatively consisting essentially of, or consisting of, irinotecan and bevacizumab, comprising, or consisting essentially of, or yet further consisting of screening a biological sample isolated from the patient for an rs1792689, rs2268753, and/or rs17776182 polymorphism, and classifying the patient as eligible for the therapy if the genotype of (G/G) for rs1792689, (C/T) or (C/C) for rs2268753, or (G/G) for rs17776182 is present in the sample. In some embodiments, the method comprises classifying the patient as not eligible for the therapy comprising, or alternatively consisting essentially of, or consisting of, irinotecan and bevacizumab if the genotype of (G/G) for rs1792689, (C/T) or (C/C) for rs2268753, or (G/G) for rs17776182 is not present in the sample. In some embodiments, the patient is classified as not eligible for the therapy comprising, or alternatively consisting essentially of, or consisting of, irinotecan and bevacizumab if the genotype of (A/G) or (A/A) for rs1792689, (T/T) for rs2268753, or (A/G) or (A/A) for rs17776182 is present in the sample. In some embodiments, the method further comprises administering a therapy comprising, or alternatively consisting essentially of, or consisting of, a therapeutically effective amount of irinotecan and bevacizumab.

Also provided, in some embodiments, are methods for increasing the progression-free and/or overall survival of a colorectal cancer patient, comprising, or alternatively consisting essentially of, or consisting of, screening a biological sample isolated from the patient for an rs1792689, rs2268753, and/or rs17776182 polymorphism, and classifying the patient as eligible for the therapy with irinotecan and bevacizumab if the genotype of (G/G) for rs1792689, (C/T) or (C/C) for rs2268753, or (G/G) for rs17776182 is present in the sample or not eligible for the therapy comprising, or alternatively consisting essentially of, or consisting of, irinotecan and bevacizumab if the genotype of (G/G) for rs1792689, (C/T) or (C/C) for rs2268753, or (G/G) for rs17776182 is not present in the sample. In some embodiments, the patient is classified as not eligible for the therapy comprising, or alternatively consisting essentially of, or consisting of, irinotecan and bevacizumab if the genotype of (A/G) or (A/A) for rs1792689, (T/T) for rs2268753, or (A/G) or (A/A) for rs17776182 is present in the sample. In some embodiments, the method further comprises administering a therapy comprising, or alternatively consisting essentially of, or consisting of, a therapeutically effective amount of irinotecan and bevacizumab or an irinotecan-free and/or bevacizumab-free therapy in accordance with the classification.

Also provided, in some embodiments, are methods for identifying whether a colorectal cancer patient is likely to experience a relatively longer or shorter progression free survival (PFS) following a therapy comprising, or alternatively consisting essentially of, or consisting of, a therapeutically effective amount of irinotecan and bevacizumab, comprising, or alternatively consisting essentially of, or consisting of, screening a biological sample isolated from the patient for an rs1792689, rs2268753, and/or rs17776182 polymorphism, and identifying that the patient is likely to experience a longer progression free survival if the genotype of (G/G) for rs1792689, (C/T) or (C/C) for rs2268753, or (G/G) for rs17776182 is present in the sample, relative to a colorectal cancer patient not having the genotype. In some embodiments, the method comprises identifying that the patient is likely to experience a shorter progression free survival if the genotype of (G/G) for rs1792689, (C/T) or (C/C) for rs2268753, or (G/G) for rs17776182 is not present in the sample, relative to a colorectal cancer patient having the genotype or relative to a colorectal cancer patient having the genotype of (A/G) or (A/A) for rs1792689, (T/T) for rs2268753, or (A/G) or (A/A) for rs17776182. In some embodiments, the method comprises identifying that the patient is likely to experience a shorter progression free survival if the genotype of (A/G) or (A/A) for rs1792689, (T/T) for rs2268753, or (A/G) or (A/A) for rs17776182 is present in the sample, relative to a colorectal cancer patient not having the genotype or relative to a colorectal cancer patient having the genotype of (G/G) for rs1792689, (C/T) or (C/C) for rs2268753, or (G/G) for rs17776182.

Provided in one embodiment, is a method for selecting a colorectal cancer patient for a therapy comprising, or consisting essentially of, or yet further consisting of irinotecan and bevacizumab, comprising, or consisting essentially of, or yet further consisting of screening a biological sample isolated from the patient for an rs7570532 and/or rs4946935 polymorphism, and selecting the patient for the therapy if the genotype of (A/A) for rs7570532 or (A/G) or (G/G) for rs4946935 is present in the sample. In some aspects, the patient is not selected for the therapy if the genotype (A/A) for rs7570532 or (A/G) or (G/G) for rs4946935 is not present in the sample. In some embodiments, the patient is not selected for therapy if the genotype of (A/G) for rs7570532 or (A/A) for rs4946935 is present in the sample. In some embodiments, the patient is selected for an irinotecan-free and/or bevacizumab-free therapy if the genotype of (A/A) for rs7570532 or (A/G) or (G/G) for rs4946935 is not present in the sample. In some embodiments, the patient is selected for an irinotecan-free and/or bevacizumab-free therapy if the genotype of (A/G) for rs7570532 or (A/A) for rs4946935 is present in the sample.

Provided in one embodiment, is a method for classifying a colorectal cancer patient as eligible for a therapy comprising, or consisting essentially of, or yet further consisting of irinotecan and bevacizumab, comprising, or consisting essentially of, or yet further consisting of screening a biological sample isolated from the patient for an rs7570532 and/or rs4946935 polymorphism, and classifying the patient as eligible for the therapy if the genotype of (A/A) for rs7570532 or (A/G) or (G/G) for rs4946935 is present in the sample.

Provided in one embodiment is a method for identifying whether a colorectal cancer patient is likely to experience a relatively longer or shorter overall survival following a therapy comprising, or consisting essentially of, or yet further consisting of irinotecan and bevacizumab, comprising, or consisting essentially of, or yet further consisting of screening a biological sample isolated from the patient for an rs7570532 and/or rs4946935 polymorphism, and identifying that the patient is likely to experience a longer overall survival if the genotype of (A/A) for rs7570532 or (A/G) or (G/G) for rs4946935 is present in the sample, relative to a colorectal cancer patient not having the genotype.

In one embodiment, provided is a method for treating a colorectal cancer patient selected for treatment based on the presence of the genotype of (A/A) for rs7570532 or (A/G) or (G/G) for rs4946935 in a biological sample from the patient, comprising, or consisting essentially of, or yet further consisting of administering to the patient a therapy comprising, or consisting essentially of, or yet further consisting of a therapeutically effective amount of irinotecan and bevacizumab, or an equivalent of each thereof. In some embodiments, the patient is treated with an irinotecan-free and/or bevacizumab-free therapy if the genotype of (A/A) for rs7570532 or (A/G) or (G/G) for rs4946935 is not present in the sample. In some embodiments, the patient is treated with an irinotecan-free and/or bevacizumab-free therapy if the genotype of (A/G) for rs7570532 or (A/A) for rs4946935 is present in the sample. In some embodiments the patient was selected by a method comprising, consisting essentially of, or yet further consisting of screening a biological sample isolated from the patient for the genotypes.

Also provided, in some embodiments, is a method for increasing the overall survival of a colorectal cancer patient, comprising, or consisting essentially of, or yet further consisting of screening a biological sample isolated from the patient for an rs7570532 and/or rs4946935 polymorphism, and classifying the patient as eligible for the therapy with irinotecan and bevacizumab if the genotype of (A/A) for rs7570532 or (A/G) or (G/G) for rs4946935 is present in the sample or not eligible for the therapy comprising irinotecan and bevacizumab if the genotype of (A/A) for rs7570532 or (A/G) or (G/G) for rs4946935 is not present in the sample. In some embodiments, the patient is classified as not eligible for the therapy comprising, or alternatively consisting essentially of, or consisting of, irinotecan and bevacizumab if the genotype of (A/G) for rs7570532 or (A/A) for rs4946935 is present in the sample. In some embodiments, the method further comprises administering a therapy comprising, or alternatively consisting essentially of, or consisting of, a therapeutically effective amount of irinotecan and bevacizumab or an irinotecan-free and/or bevacizumab-free therapy in accordance with the classification.

Also provided, in some embodiments, are methods for treating a colorectal cancer patient selected for treatment based on the presence of the genotype of (G/G) for rs1792689, (C/T) or (C/C) for rs2268753, or (G/G) for rs17776182 in a biological sample from the patient, comprising, or alternatively consisting essentially of, or yet further consists of, administering to the patient a therapy comprising, or alternatively consisting essentially of, or yet further consists of, a therapeutically effective amount of irinotecan and bevacizumab. Also provided, in some embodiments, are methods for treating a colorectal cancer patient selected for treatment based on the absence of the genotype of (A/G) or (A/A) for rs1792689, (T/T) for rs2268753, or (A/G) or (A/A) for rs17776182 in a biological sample from the patient, comprising, or alternatively consisting essentially of, or yet further consists of, administering to the patient a therapy comprising, or alternatively consisting essentially of, or yet further consists of, a therapeutically effective amount of a therapeutically effective amount of irinotecan and bevacizumab.

In some embodiments, the method further comprises screening a biological sample isolated from the patient for the rs1792689, rs2268753, and/or rs17776182 polymorphism. Thus, also provided, in some embodiments, are methods for treating a colorectal cancer patient, comprising, or alternatively consisting essentially of, or yet further consists of, screening a biological sample isolated from the patient for rs1792689, rs2268753, and/or rs17776182 polymorphism and administering to the patient a therapy comprising, or alternatively consisting essentially of, or yet further consists of, a therapeutically effective amount of irinotecan and bevacizumab if the sample has the genotype of (G/G) for rs1792689, (C/T) or (C/C) for rs2268753, or (G/G) for rs17776182.

Also provided, in some embodiments, are methods for modifying the treatment of patient receiving a therapy comprising, or alternatively consisting essentially of, or yet further consists of, a therapeutically effective amount of irinotecan and bevacizumab based on the presence of the genotype of (G/G) for rs1792689, (C/T) or (C/C) for rs2268753, or (G/G) for rs17776182 in a biological sample from the patient. For example, provided are methods for modifying the treatment of patient receiving a therapy comprising, or alternatively consisting essentially of, or yet further consists of, a therapeutically effective amount of irinotecan and bevacizumab, comprising, or alternatively consisting essentially of, or yet further consists of, screening a biological sample isolated from the patient for an rs1792689, rs2268753, and/or rs17776182 polymorphism, and modifying the dosage or frequency of the therapy comprising, or alternatively consisting essentially of, or yet further consists of, a therapeutically effective amount of irinotecan and bevacizumab based on the genotype for rs1792689, rs2268753, and/or rs17776182. In some embodiments, the dosage or frequency of the therapy, or components thereof (e.g., one or more therapeutic agents of the therapy), is increased if the genotype of (G/G) for rs1792689, (C/T) or (C/C) for rs2268753, or (G/G) for rs17776182 is not present in the sample. In some embodiments, the dosage or frequency of the therapy, or components thereof, is increased if the genotype of (A/G) or (A/A) for rs1792689, (T/T) for rs2268753, or (A/G) or (A/A) for rs17776182 is present in the sample. In some embodiments, the therapy is discontinued if the genotype of (G/G) for rs1792689, (C/T) or (C/C) for rs2268753, or (G/G) for rs17776182 is not present in the sample. In some embodiments, the therapy is discontinued if the genotype of (A/G) or (A/A) for rs1792689, (T/T) for rs2268753, or (A/G) or (A/A) for rs17776182 is present in the sample. In some embodiments, the therapy is continued if the genotype of (G/G) for rs1792689, (C/T) or (C/C) for rs2268753, or (G/G) for rs17776182 is present in the sample.

Also provided, in some embodiments, are methods for modifying the treatment of patient receiving a therapy comprising, or alternatively consisting essentially of, or yet further consists of, a therapeutically effective amount of irinotecan and bevacizumab based on the presence of the genotype of (A/A) for rs7570532 or (A/G) or (G/G) for rs4946935 in a biological sample from the patient. For example, provided are methods for modifying the treatment of patient receiving a therapy comprising, or alternatively consisting essentially of, or yet further consists of, a therapeutically effective amount of irinotecan and bevacizumab, comprising, or alternatively consisting essentially of, or yet further consists of, screening a biological sample isolated from the patient for an rs7570532, and/or rs4946935 polymorphism, and modifying the dosage or frequency of the therapy comprising, or alternatively consisting essentially of, or yet further consists of, a therapeutically effective amount of irinotecan and bevacizumab based on the genotype for rs7570532, and/or rs4946935. In some embodiments, the dosage or frequency of the therapy, or components thereof (e.g., one or more therapeutic agents of the therapy), is increased if the genotype of (A/A) for rs7570532 or (A/G) or (G/G) for rs4946935 is not present in the sample. In some embodiments, the dosage or frequency of the therapy, or components thereof, is increased if the genotype of (A/G) for rs7570532 or (A/A) for rs4946935 is present in the sample. In some embodiments, the therapy is discontinued if the genotype of (A/A) for rs7570532 or (A/G) or (G/G) for rs4946935 is not present in the sample. In some embodiments, the therapy is discontinued if the genotype of (A/G) for rs7570532 or (A/A) for rs4946935 is present in the sample. In some embodiments, the therapy is continued if the genotype of (A/A) for rs7570532 or (A/G) or (G/G) for rs4946935 is present in the sample.

In some embodiments, screening a biological sample isolated from the patient for an rs1792689, rs2268753, rs17776182, rs7570532 and/or rs4946935 polymorphism comprises contacting the biological sample with a nucleic acid probe that specifically binds to nucleic acid containing the rs1792689, rs2268753, rs17776182, rs7570532 and/or rs4946935 polymorphism and overlaps the polymorphic site. For example, in some embodiments, the nucleic acid specifically binds to a nucleic acid having the sequence of any of SEQ ID NO: 1-3, 17 and 18 and overlaps the polymorphic site. In some embodiments, the nucleic acid is labeled with a detectable moiety. Having about 5, about 10, about 15, about 20, about 25, about 30, about 35, or about 40 nucleotides upstream and/or downstream of the polymorphic region.

In some embodiments, screening a biological sample isolated from the patient for an rs1792689, rs2268753, rs17776182, rs7570532 and/or rs4946935 polymorphism comprises amplifying nucleic acid containing the rs1792689, rs2268753, rs17776182, rs7570532 and/or rs4946935 polymorphism. In some embodiments, nucleic acid containing the rs1792689, rs2268753, rs17776182, rs7570532 and/or rs4946935 polymorphism is amplified using a forward primer and a reverse primer that flank each polymorphism. For example, nucleic acid containing the rs1792689 polymorphism is amplified using a forward primer comprising, or consisting essentially of, or yet further consisting of nucleic acid having the sequence of SEQ ID NO: 4 and a reverse primer comprising, or consisting essentially of, or yet further consisting of nucleic acid having the sequence of SEQ ID NO: 5, nucleic acid containing the rs2268753 polymorphism is amplified using a forward primer comprising, or consisting essentially of, or yet further consisting of nucleic acid having the sequence of SEQ ID NO: 6 and a reverse primer comprising, or consisting essentially of, or yet further consisting of nucleic acid having the sequence of SEQ ID NO: 7, and/or nucleic acid containing the rs17776182 polymorphism is amplified using a forward primer comprising, or consisting essentially of, or yet further consisting of nucleic acid having the sequence of SEQ ID NO: 8 and a reverse primer comprising, or consisting essentially of, or yet further consisting of nucleic acid having the sequence of SEQ ID NO: 9. For example, nucleic acid containing the rs7570532 polymorphism is amplified using a forward primer comprising, or consisting essentially of, or yet further consisting of nucleic acid having the sequence of SEQ ID NO: 13 and a reverse primer comprising, or consisting essentially of, or yet further consisting of nucleic acid having the sequence of SEQ ID NO: 14, and/or nucleic acid containing the rs4946935 polymorphism is amplified using a forward primer comprising, or consisting essentially of, or yet further consisting of nucleic acid having the sequence of SEQ ID NO: 15 and a reverse primer comprising, or consisting essentially of, or yet further consisting of nucleic acid having the sequence of SEQ ID NO: 16.

In some aspects, therapy comprising, or alternatively consisting essentially of, or yet further consists of, irinotecan and bevacizumab further comprises, or alternatively consisting essentially of, or yet further consists of, a therapeutically effective amount of folinic acid and/or a pyrimidine analog. In some aspects, the therapy comprises FOLFIRI (leucovorin+Fluorouracil (5-FU)+irinotecan). In some aspects, the therapy comprises therapeutically effective amounts of irinotecan and oxaliplatin. In some aspects, the therapy comprises FOLFOXFIRI (leucovorin+Fluorouracil (5-FU)+oxaliplatin+irinotecan).

In some aspects, the patient has a wild-type KRAS and/or BRAF gene.

In some aspects, the patient suffers from non-metastatic colorectal cancer or metastatic colorectal cancer.

In some aspects, the biological sample is a tissue or a cell sample. In some aspects, the sample comprises at least one of a tumor cell, a normal cell adjacent to a tumor, a normal cell corresponding to the tumor tissue type, a blood cell, a peripheral blood lymphocyte, or combinations thereof.

In some aspects, the sample is at least one of blood, plasma, serum, an original sample recently isolated from the patient, a fixed tissue, a frozen tissue, a biopsy tissue, a resection tissue, a microdissected tissue, or combinations thereof.

In some aspects, the screening the rs1792689, rs2268753, rs17776182, rs7570532 and/or rs4946935 polymorphism is by a method comprising, or consisting essentially of, or yet further consisting of PCR, RT-PCR, real-time PCR, PCR-RFLP, sequencing, or nucleic probe hybridization in solution or on a solid support, such as a chip or a microarray. In some aspects, the patient is a human patient.

Also provided, in some embodiments, are kits for screening for selecting a colorectal cancer patient for a therapy comprising, or alternatively consisting essentially of, or yet further consists of, irinotecan and bevacizumab or for classifying a colorectal cancer patient as eligible for a therapy comprising, or alternatively consisting essentially of, or yet further consists of, irinotecan and bevacizumab. In some embodiments, the kit comprises primer for amplification of nucleic acid containing a rs1792689, rs2268753, rs17776182, rs7570532 and/or rs4946935 polymorphism. In some embodiments, the kit comprises a nucleic acid probe that specifically binds to nucleic acid containing the rs1792689, rs2268753, and/or rs17776182 polymorphism and overlaps the polymorphic site. In some embodiments, the kit comprises a nucleic acid probe that specifically binds to nucleic acid containing the rs7570532 and/or rs4946935 polymorphism and overlaps the polymorphic site. For example, in some embodiments, the nucleic acid probe specifically binds to a nucleic acid having the sequence of any of SEQ ID NO: 1-3, 17 and 18 and overlaps the polymorphic site. In some embodiments, the nucleic acid probe has about 5, about 10, about 15, about 20, about 25, about 30, about 35 or about 40 or more contiguous nucleotides of any of SEQ ID NO: 1-3, 17 and 18 and overlaps the polymorphic site.

### BRIEF DESCRIPTION OF THE DRAWINGS

FIGS. 1A-1B: Kaplan-Meier cumulative overall survival probability curves stratified by ACVR2B rs2268753 genotype in the training cohort (FIG. 2A) and control cohort (FIG. 2B) in RAS mutant patients.

FIGS. 2A-2B: Activin/myostatin signaling in the skeletal muscle (FIG. 2A) and cancer cell (FIG. 2B). Referring to FIG. 2A activin or myostatin binds to type IIB activin receptor (ACVR2B) on the muscle membrane, resulting in the phosphorylation and recruitment of type I activin receptor transmembrane kinase, ALK4 or ALK5. This binding induces the COOH-terminal phosphorylation of Smad2 and Smad3 and the recruitment of Smad4 into a Smad complex. The Smad complex translocates into the nucleus to elicit transcriptional changes, which result in muscle wasting. Activin/myostatin binding to the receptor also suppresses Akt activity and consequently reduces FOXO phosphorylation. Dephosphorylated FOXOs can enter the nucleus to activate transcription of ubiquitine proteosome E3 ligase (MuRF1, Atrogin-1), and other atrogenes. Referring to FIG. 2B, in tumor cells, activated RAS signaling induces the phosphorylation of Smad2 and Smad3 at the linker region, and dually phosphorylated Smad2 or Smad3 upregulates the transcription of c-Myc and MMP-9.

### DETAILED DESCRIPTION

Throughout this disclosure, various publications, patents and published patent specifications are referenced by an identifying citation. The disclosures of these publications, patents and published patent specifications are hereby incorporated by reference into the present disclosure to more fully describe the state of the art to which this disclosure pertains.

As used herein, certain terms may have the following defined meanings. As used in the specification and claims, the singular form “a,” “an” and “the” include singular and plural references unless the context clearly dictates otherwise. For example, the term “a cell” includes a single cell as well as a plurality of cells, including mixtures thereof.

As used herein, the term “comprising” is intended to mean that the methods include the recited elements, but not excluding others. “Consisting essentially of” when used to define methods, shall mean excluding other elements of any essential significance to the method. “Consisting of” shall mean excluding more than trace elements of other ingredients for claimed compositions and substantial method steps. Embodiments defined by each of these transition terms are within the scope of this disclosure. Accordingly, it is intended that the methods can include additional steps and components (comprising) or alternatively including steps of no significance (consisting essentially of) or alternatively, intending only the stated method steps (consisting of).

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about”. The term “about” also includes the exact value “X” in addition to minor increments of “X” such as “X+0.1” or “X—0.1.” It also is to be understood, although not always explicitly stated, that the reagents described herein are merely exemplary and that equivalents of such are known in the art.

The term “chemotherapy” encompasses cancer therapies that employ chemical or biological agents or other therapies, such as radiation therapies, e.g., a small molecule drug or a large molecule, such as antibodies, RNAi and gene therapies. Non-limiting examples of chemotherapies are provided below. Unless specifically excluded, when a specific therapy is recited, equivalents of the therapy are within the scope of this invention.

Bevacizumab (BV) is sold under the trade name Avastin® by Genentech. It is a humanized monoclonal antibody that binds to and inhibits the biologic activity of human vascular endothelial growth factor (VEGF). Biological equivalent antibodies are identified herein as modified antibodies which bind to the same epitope of the antigen, prevent the interaction of VEGF to its receptors (Flt01, KDR a.k.a. VEGFR2) and produce a substantially equivalent response, e.g., the blocking of endothelial cell proliferation and angiogenesis. Irinotecan and bevacizumab is also in the class of cancer drugs that inhibit angiogenesis (angiogenesis inhibitors).

Irinotecan (CPT-11) is sold under the trade name of Camptosar®. It is a semi-synthetic analogue of the alkaloid camptothecin, which is activated by hydrolysis to SN-38 and targets topoisomerase I. Chemical equivalents are those that inhibit the interaction of topoisomerase I and DNA to form a catalytically active topoisomerase I-DNA complex. Chemical equivalents inhibit cell cycle progression at G2-M phase resulting in the disruption of cell proliferation. An equivalent of irinotecan is a composition that inhibits a topoisomerase. Non-limiting examples of an equivalent of irinotecan include topotecan, camptothecin and lamellarin D, etoposide, or doxorubicin.

Oxaliplatin (trans-/-diaminocyclohexane oxalatoplatinum; L-OHP; CAS No. 61825-94-3) is sold under the trade name of Elotaxin. It is a platinum derivative that causes cell cytotoxicity. Oxaliplatin forms both inter- and intra-strand cross links in DNA, which prevent DNA replication and transcription, causing cell death. Non-limiting examples of an equivalent of oxaliplatin include carboplatin and cisplatin.

Topoisomerase inhibitors are agents designed to interfere with the action of topoisomerase enzymes (topoisomerase I and II), which are enzymes that control the changes in DNA structure by catalyzing the breaking and rejoining of the phosphodiester backbone of DNA strands during the normal cell cycle. In one aspect, topoisomerase inhibitors include irinotecan, topotecan, camptothecin and lamellarin D, or compounds targeting topoisomerase IA. In another aspect, topoisomerase inhibitors include etoposide, doxorubicin or compounds targeting topoisomerase II.

Pyrimidine antimetabolite includes, without limitation, fluorouracil (5-FU), its equivalents and prodrugs. In one embodiment, a pyrimidine antimetabolite is a chemical that inhibits the use of a pyrimidine. The presence of antimetabolites can have toxic effects on cells, such as halting cell growth and cell division, so these compounds can be used as chemotherapy for cancer.

Fluorouracil (5-FU) belongs to the family of therapy drugs called pyrimidine based anti-metabolites. It is a pyrimidine analog, which is transformed into different cytotoxic metabolites that are then incorporated into DNA and RNA thereby inducing cell cycle arrest and apoptosis. Chemical equivalents are pyrimidine analogs which result in disruption of DNA replication. Chemical equivalents inhibit cell cycle progression at S phase resulting in the disruption of cell cycle and consequently apoptosis. Equivalents to 5-FU include prodrugs, analogs and derivative thereof such as 5′-deoxy-5-fluorouridine (doxifluroidine), 1-tetrahydrofuranyl-5-fluorouracil (ftorafur), Capecitabine (Xeloda), S-1 (MBMS-247616, consisting of tegafur and two modulators, a 5-chloro-2,4-dihydroxypyridine and potassium oxonate), ralititrexed (tomudex), nolatrexed (Thymitaq, AG337), LY231514 and ZD9331, as described for example in Papamicheal (1999) The Oncologist 4:478-487.

“5-FU based adjuvant therapy” refers to 5-FU alone or alternatively the combination of 5-FU with other treatments, that include, but are not limited to radiation, methyl-CCNU, leucovorin, oxaliplatin, irinotecin, mitomycin, cytarabine, levamisole. Specific treatment adjuvant regimens are known in the art as FOLFOX, FOLFOX4, FOLFIRI, MOF (semustine (methyl-CCNU), vincrisine (Oncovin) and 5-FU). For a review of these therapies see Beaven and Goldberg (2006) Oncology 20(5):461-470. An example of such is an effective amount of 5-FU and Leucovorin. Other chemotherapeutics can be added, e.g., oxaliplatin or irinotecan.

Capecitabine is a prodrug of (5-FU) that is converted to its active form by the tumor-specific enzyme PynPase following a pathway of three enzymatic steps and two intermediary metabolites, 5′-deoxy-5-fluorocytidine (5′-DFCR) and 5′-deoxy-5-fluorouridine (5′-DFUR). Capecitabine is marketed by Roche under the trade name Xeloda®.

A therapy comprising, or consisting essentially of, or yet further consisting of a pyrimidine antimetabolite includes, without limitation, a pyrimidine antimetabolite alone or alternatively the combination of a pyrimidine antimetabolite with other treatments, that include, but are not limited to, radiation, methyl-CCNU, leucovorin, oxaliplatin, irinotecin, mitomycin, cytarabine, levamisole. Specific treatment adjuvant regimens are known in the art as FOLFOX, FOLFOX4, FOLFOX6, FOLFIRI, MOF (semustine (methyl-CCNU), vincrisine (Oncovin) and 5-FU). For a review of these therapies see Beaven and Goldberg (2006) Oncology 20(5):461-470. An example of such is an effective amount of 5-FU and Leucovorin. Other chemotherapeutics can be added, e.g., oxaliplatin or irinotecan.

FOLFIRI is a chemotherapy regimen for treatment of colorectal cancer. It is made up of the following drugs: FOL—folinic acid (leucovorin), a vitamin B derivative used as a “rescue” drug for high doses of the drug methotrexate and that modulates/potentiates/reduces the side effects of fluorouracil; F—fluorouracil (5-FU), a pyrimidine analog and antimetabolite which incorporates into the DNA molecule and stops synthesis; and IRI—irinotecan (Camptosar), a topoisomerase inhibitor, which prevents DNA from uncoiling and duplicating.

FOLFOX is a chemotherapy regimen for treatment of colorectal cancer. is made up of the following drugs: FOL—folinic acid (leucovorin), F—fluorouracil (5-FU), and OX—oxaliplatin.

FOLFOXFIRI is a chemotherapy regimen for treatment of colorectal cancer. It is made up of the following drugs: FOL—folinic acid (leucovorin), F—fluorouracil (5-FU), OX—oxaliplatin and IRI—irinotecan (Camptosar).

The phrase “first line” or “second line” or “third line” refers to the order of treatment received by a patient. First line therapy regimens are treatments given first, whereas second or third line therapy are given after the first line therapy or after the second line therapy, respectively. The National Cancer Institute defines first line therapy as “the first treatment for a disease or condition. In patients with cancer, primary treatment can be surgery, chemotherapy, radiation therapy, or a combination of these therapies. First line therapy is also referred to those skilled in the art as “primary therapy and primary treatment.” See National Cancer Institute website at cancer.gov. Typically, a patient is given a subsequent chemotherapy regimen because the patient did not shown a positive clinical or sub-clinical response to the first line therapy or the first line therapy has stopped.

In one aspect, the term “equivalent” or “biological equivalent” of an antibody means the ability of the antibody to selectively bind its epitope protein or fragment thereof as measured by ELISA or other suitable methods. Biologically equivalent antibodies include, but are not limited to, those antibodies, peptides, antibody fragments, antibody variant, antibody derivative and antibody mimetics that bind to the same epitope as the reference antibody.

In one aspect, the term “equivalent” of “chemical equivalent” of a chemical means the ability of the chemical to selectively interact with its target protein, DNA, RNA or fragment thereof as measured by the inactivation of the target protein, incorporation of the chemical into the DNA or RNA or other suitable methods. Chemical equivalents include, but are not limited to, those agents with the same or similar biological activity and include, without limitation a pharmaceutically acceptable salt or mixtures thereof that interact with and/or inactivate the same target protein, DNA, or RNA as the reference chemical.

The term “allele,” which is used interchangeably herein with “allelic variant” refers to alternative forms of a gene or portions thereof. Alleles occupy the same locus or position on homologous chromosomes. When a subject has two identical alleles of a gene, the subject is said to be homozygous for the gene or allele. When a subject has two different alleles of a gene, the subject is said to be heterozygous for the gene. Alleles of a specific gene can differ from each other in a single nucleotide, or several nucleotides, and can include substitutions, deletions and insertions of nucleotides. An allele of a gene can also be a form of a gene containing a mutation.

As used herein, the term “determining the genotype of a cell or tissue sample” intends to identify the genotypes of polymorphic loci of interest in the cell or tissue sample. In one aspect, a polymorphic locus is a single nucleotide polymorphic (SNP) locus. If the allelic composition of a SNP locus is heterozygous, the genotype of the SNP locus will be identified as “X/Y” wherein X and Y are two different nucleotides. If the allelic composition of a SNP locus is heterozygous, the genotype of the SNP locus will be identified as “X/X” wherein X identifies the nucleotide that is present at both alleles.

The term “genetic marker” refers to an allelic variant of a polymorphic region of a gene of interest and/or the expression level of a gene of interest.

The term “polymorphism” refers to the coexistence of more than one form of a gene or portion thereof. A portion of a gene of which there are at least two different forms, i.e., two different nucleotide sequences, is referred to as a “polymorphic region of a gene.” A polymorphic region can be a single nucleotide, the identity of which differs in different alleles.

The term “genotype” refers to the specific allelic composition of an entire cell or a certain gene and in some aspects a specific polymorphism associated with that gene, whereas the term “phenotype” refers to the detectable outward manifestations of a specific genotype.

The terms “KRAS wild-type” and “BRAF wild-type” refers to a genotype of a cell or patient in which no mutation is detected in the corresponding gene. In some aspects, no mutation is detected that affects the function or activity of the gene.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

The term “isolated” as used herein refers to molecules or biological or cellular materials being substantially free from other materials. In one aspect, the term “isolated” refers to nucleic acid, such as DNA or RNA, or protein or polypeptide, or cell or cellular organelle, or tissue or organ, separated from other DNAs or RNAs, or proteins or polypeptides, or cells or cellular organelles, or tissues or organs, respectively, that are present in the natural source. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides. The term “isolated” is also used herein to refer to cells or tissues that are isolated from other cells or tissues and is meant to encompass both cultured and engineered cells or tissues.

The term “treating” as used herein is intended to encompass curing as well as ameliorating at least one symptom of the condition or disease. For example, in the case of cancer, a response to treatment includes a reduction in cachexia, increase in survival time, elongation in time to tumor progression, reduction in tumor mass, reduction in tumor burden and/or a prolongation in time to tumor metastasis, time to tumor recurrence, tumor response, complete response, partial response, stable disease, progressive disease, progression free survival, overall survival, each as measured by standards set by the National Cancer Institute and the U.S. Food and Drug Administration for the approval of new drugs.

“An effective amount” or “therapeutically effect amount” intends to indicate the amount of a compound or agent administered or delivered to the patient which is most likely to result in the desired response to treatment. The amount is empirically determined by the patient's clinical parameters including, but not limited to the stage of disease, age, gender, histology, and likelihood for tumor recurrence. These terms may be used interchangeably.

A “patient” as used herein intends an animal patient, a mammal patient or yet further a human patient. For the purpose of illustration only, a mammal includes but is not limited to a simian, a murine, a bovine, an equine, a porcine or an ovine subject.

The term “clinical outcome”, “clinical parameter”, “clinical response”, or “clinical endpoint” refers to any clinical observation or measurement relating to a patient's reaction to a therapy. Non-limiting examples of clinical outcomes include tumor response (TR), overall survival (OS), progression free survival (PFS), disease free survival, time to tumor recurrence (TTR), time to tumor progression (TTP), relative risk (RR), toxicity or side effect.

The term “suitable for a therapy” or “suitably treated with a therapy” shall mean that the patient is likely to exhibit one or more desirable clinical outcomes as compared to patients having the same disease and receiving the same therapy but possessing a different characteristic that is under consideration for the purpose of the comparison. In one aspect, the characteristic under consideration is a genetic polymorphism or a somatic mutation. In another aspect, the characteristic under consideration is expression level of a gene or a polypeptide. In one aspect, a more desirable clinical outcome is relatively higher likelihood of or relatively better tumor response such as tumor load reduction. In another aspect, a more desirable clinical outcome is relatively longer overall survival. In yet another aspect, a more desirable clinical outcome is relatively longer progression free survival or time to tumor progression. In yet another aspect, a more desirable clinical outcome is relatively longer disease free survival. In further another aspect, a more desirable clinical outcome is relative reduction or delay in tumor recurrence. In another aspect, a more desirable clinical outcome is relatively decreased metastasis. In another aspect, a more desirable clinical outcome is relatively lower relative risk. In yet another aspect, a more desirable clinical outcome is relatively reduced toxicity or side effects. In some embodiments, more than one clinical outcomes are considered simultaneously. In one such aspect, a patient possessing a characteristic, such as a genotype of a genetic polymorphism, can exhibit more than one more desirable clinical outcomes as compared to patients having the same disease and receiving the same therapy but not possessing the characteristic. As defined herein, the patient is considered suitable for the therapy. In another such aspect, a patient possessing a characteristic can exhibit one or more desirable clinical outcome but simultaneously exhibit one or more less desirable clinical outcome. The clinical outcomes will then be considered collectively, and a decision as to whether the patient is suitable for the therapy will be made accordingly, taking into account the patient's specific situation and the relevance of the clinical outcomes. In some embodiments, progression free survival or overall survival is weighted more heavily than tumor response in a collective decision making.

A “tumor response” (TR) refers to a tumor's response to therapy. A “complete response” (CR) to a therapy refers to the clinical status of a patient with evaluable but non-measurable disease, whose tumor and all evidence of disease have disappeared following administration of the therapy. In this context, a “partial response”(PR) refers to a response that is anything less than a complete response. “Stable disease” (SD) indicates that the patient is stable following the therapy. “Progressive disease” (PD) indicates that the tumor has grown (i.e. become larger) or spread (i.e. metastasized to another tissue or organ) or the overall cancer has gotten worse following the therapy. For example, tumor growth of more than 20 percent since the start of therapy typically indicates progressive disease. “Non-response” (NR) to a therapy refers to status of a patient whose tumor or evidence of disease has remained constant or has progressed.

“Overall Survival” (OS) refers to the length of time of a cancer patient remaining alive following a cancer therapy.

“Progression free survival” (PFS) or “Time to Tumor Progression” (TTP) refers to the length of time following a therapy, during which the tumor in a cancer patient does not grow. Progression-free survival includes the amount of time a patient has experienced a complete response, partial response or stable disease.

“Disease free survival” refers to the length of time following a therapy, during which a cancer patient survives with no signs of the cancer or tumor.

“Time to Tumor Recurrence (TTR)” refers to the length of time, following a cancer therapy such as surgical resection or chemotherapy, until the tumor has reappeared (come back). The tumor may come back to the same place as the original (primary) tumor or to another place in the body.

“Relative Risk” (RR), in statistics and mathematical epidemiology, refers to the risk of an event (or of developing a disease) relative to exposure. Relative risk is a ratio of the probability of the event occurring in the exposed group versus a non-exposed group.

The term “identify” or “identifying” is to associate or affiliate a patient closely to a group or population of patients who likely experience the same or a similar clinical response to a therapy.

The term “selecting” a patient for a therapy refers to making an indication that the selected patient is suitable for the therapy. Such an indication can be made in writing by, for instance, a handwritten prescription or a computerized report making the corresponding prescription or recommendation.

When a genetic marker or polymorphism “is used as a basis” for identifying or selecting a patient for a treatment described herein, the genetic marker or polymorphism is measured before and/or during treatment, and the values obtained are used by a clinician in assessing any of the following: (a) probable or likely suitability of an individual to initially receive treatment(s); (b) probable or likely unsuitability of an individual to initially receive treatment(s); (c) responsiveness to treatment; (d) probable or likely suitability of an individual to continue to receive treatment(s); (e) probable or likely unsuitability of an individual to continue to receive treatment(s); (f) adjusting dosage; (g) predicting likelihood of clinical benefits; or (h) toxicity. As would be well understood by one in the art, measurement of the genetic marker or polymorphism in a clinical setting is a clear indication that this parameter was used as a basis for initiating, continuing, adjusting and/or ceasing administration of the treatments described herein.

“Having the same cancer” is used when comparing one patient to another or alternatively, one patient population to another patient population. For example, the two patients or patient population will each have or be suffering from colon cancer.

A “normal cell corresponding to the tumor tissue type” refers to a normal cell from a same tissue type as the tumor tissue. A non-limiting examples is a normal lung cell from a patient having lung tumor, or a normal colon cell from a patient having colon tumor.

The term “amplification” or “amplify” as used herein means one or more methods known in the art for copying a target nucleic acid, thereby increasing the number of copies of a selected nucleic acid sequence. Amplification can be exponential or linear. A target nucleic acid can be either DNA or RNA. The sequences amplified in this manner form an “amplicon.” While the exemplary methods described hereinafter relate to amplification using the polymerase chain reaction (“PCR”), numerous other methods are known in the art for amplification of nucleic acids (e.g., isothermal methods, rolling circle methods, etc.). The skilled artisan will understand that these other methods can be used either in place of, or together with, PCR methods.

The term “complement” as used herein means the complementary sequence to a nucleic acid according to standard Watson/Crick base pairing rules. A complement sequence can also be a sequence of RNA complementary to the DNA sequence or its complement sequence, and can also be a cDNA. The term “substantially complementary” as used herein means that two sequences hybridize under stringent hybridization conditions. The skilled artisan will understand that substantially complementary sequences need not hybridize along their entire length. In particular, substantially complementary sequences comprise a contiguous sequence of bases that do not hybridize to a target or marker sequence, positioned 3′ or 5′ to a contiguous sequence of bases that hybridize under stringent hybridization conditions to a target or marker sequence.

As used herein, the term “hybridize” or “specifically hybridize” refers to a process where two complementary nucleic acid strands anneal to each other under appropriately stringent conditions. Hybridizations are typically conducted with probe-length nucleic acid molecules. Nucleic acid hybridization techniques are well known in the art. Those skilled in the art understand how to estimate and adjust the stringency of hybridization conditions such that sequences having at least a desired level of complementarity will stably hybridize, while those having lower complementarity will not. For examples of hybridization conditions and parameters, see, e.g., Sambrook, et al., 1989, Molecular Cloning: A Laboratory Manual, Second Edition, Cold Spring Harbor Press, Plainview, N.Y.; Ausubel, F. M. et al. 1994, Current Protocols in Molecular Biology. John Wiley & Sons, Secaucus, N.J.

“Primer” as used herein refers to an oligonucleotide that is capable of acting as a point of initiation of synthesis when placed under conditions in which primer extension is initiated (e.g., primer extension associated with an application such as PCR). The primer is complementary to a target nucleotide sequence and it hybridizes to a substantially complementary sequence in the target and leads to addition of nucleotides to the 3′-end of the primer in the presence of a DNA or RNA polymerase. The 3′-nucleotide of the primer should generally be complementary to the target sequence at a corresponding nucleotide position for optimal expression and amplification. An oligonucleotide “primer” can occur naturally, as in a purified restriction digest or can be produced synthetically. The term “primer” as used herein includes all forms of primers that can be synthesized including, peptide nucleic acid primers, locked nucleic acid primers, phosphorothioate modified primers, labeled primers, and the like.

Primers are typically between about 5 and about 100 nucleotides in length, such as between about 15 and about 60 nucleotides in length, such as between about 20 and about 50 nucleotides in length, such as between about 25 and about 40 nucleotides in length. In some embodiments, primers can be at least 8, at least 12, at least 16, at least 20, at least 25, at least 30, at least 35, at least 40, at least 45, at least 50, at least 55, at least 60 nucleotides in length. An optimal length for a particular primer application can be readily determined in the manner described in H. Erlich, PCR Technology. Principles and Application for DNA Amplification (1989).

“Probe” as used herein refers to nucleic acid that interacts with a target nucleic acid via hybridization. A probe can be fully complementary to a target nucleic acid sequence or partially complementary. The level of complementarity will depend on many factors based, in general, on the function of the probe. A probe or probes can be used, for example to detect the presence or absence of a mutation in a nucleic acid sequence by virtue of the sequence characteristics of the target. Probes can be labeled or unlabeled, or modified in any of a number of ways well known in the art. A probe can specifically hybridize to a target nucleic acid.

Probes can be DNA, RNA or a RNA/DNA hybrid. Probes can be oligonucleotides, artificial chromosomes, fragmented artificial chromosome, genomic nucleic acid, fragmented genomic nucleic acid, RNA, recombinant nucleic acid, fragmented recombinant nucleic acid, peptide nucleic acid (PNA), locked nucleic acid, oligomer of cyclic heterocycles, or conjugates of nucleic acid. Probes can comprise modified nucleobases, modified sugar moieties, and modified internucleotide linkages. A probe can be fully complementary to a target nucleic acid sequence or partially complementary. A probe can be used to detect the presence or absence of a target nucleic acid. Probes are typically at least about 10, 15, 21, 25, 30, 35, 40, 50, 60, 75, 100 nucleotides or more in length.

“Detecting” as used herein refers to determining the presence of a nucleic acid of interest in a sample or the presence of a protein of interest in a sample. Detection does not require the method to provide 100% sensitivity and/or 100% specificity.

“Detectable label” as used herein refers to a molecule or a compound or a group of molecules or a group of compounds used to identify a nucleic acid or protein of interest. In some cases, the detectable label can be detected directly. In other cases, the detectable label can be a part of a binding pair, which can then be subsequently detected. Signals from the detectable label can be detected by various means and will depend on the nature of the detectable label. Detectable labels can be isotopes, fluorescent moieties, colored substances, and the like. Examples of means to detect detectable label include but are not limited to spectroscopic, photochemical, biochemical, immunochemical, electromagnetic, radiochemical, or chemical means, such as fluorescence, chemifluorescence, or chemiluminescence, or any other appropriate means.

“TaqMan® PCR detection system” as used herein refers to a method for real time PCR. In this method, a TaqMan® probe which hybridizes to the nucleic acid segment amplified is included in the PCR reaction mix. The TaqMan® probe comprises a donor and a quencher fluorophore on either end of the probe and in close enough proximity to each other so that the fluorescence of the donor is taken up by the quencher. However, when the probe hybridizes to the amplified segment, the 5′-exonuclease activity of the Taq polymerase cleaves the probe thereby allowing the donor fluorophore to emit fluorescence which can be detected.

As used herein, the term “sample” or “test sample” refers to any liquid or solid material containing nucleic acids. In suitable embodiments, a test sample is obtained from a biological source (i.e., a “biological sample”), such as cells in culture or a tissue sample from an animal, preferably, a human. In an exemplary embodiment, the sample is a biopsy sample.

“Target nucleic acid” as used herein refers to segments of a chromosome, a complete gene with or without intergenic sequence, segments or portions a gene with or without intergenic sequence, or sequence of nucleic acids to which probes or primers are designed. Target nucleic acids can include wild type sequences, nucleic acid sequences containing mutations, deletions or duplications, tandem repeat regions, a gene of interest, a region of a gene of interest or any upstream or downstream region thereof. Target nucleic acids can represent alternative sequences or alleles of a particular gene. Target nucleic acids can be derived from genomic DNA, cDNA, or RNA. As used herein, target nucleic acid can be native DNA or a PCR-amplified product.

As used herein the term “stringency” is used in reference to the conditions of temperature, ionic strength, and the presence of other compounds, under which nucleic acid hybridizations are conducted. With high stringency conditions, nucleic acid base pairing will occur only between nucleic acids that have sufficiently long segments with a high frequency of complementary base sequences. Exemplary hybridization conditions are as follows. High stringency generally refers to conditions that permit hybridization of only those nucleic acid sequences that form stable hybrids in 0.018 M NaCl at 65° C. High stringency conditions can be provided, for example, by hybridization in 50% formamide, 5×Denhardt's solution, 5×SSC (saline sodium citrate) 0.2% SDS (sodium dodecyl sulfate) at 42° C., followed by washing in 0.1×SSC, and 0.1% SDS at 65° C. Moderate stringency refers to conditions equivalent to hybridization in 50% formamide, 5×Denhardt's solution, 5×SSC, 0.2% SDS at 42° C., followed by washing in 0.2×SSC, 0.2% SDS, at 65° C. Low stringency refers to conditions equivalent to hybridization in 10% formamide, 5×Denhardt's solution, 6×SSC, 0.2% SDS, followed by washing in 1×SSC, 0.2% SDS, at 50° C.

As used herein the term “substantially identical” refers to a polypeptide or nucleic acid exhibiting at least 50%, 75%, 85%, 90%, 95%, or even 99% identity to a reference amino acid or nucleic acid sequence over the region of comparison. For polypeptides, the length of comparison sequences will generally be at least 20, 30, 40, or 50 amino acids or more, or the full length of the polypeptide. For nucleic acids, the length of comparison sequences will generally be at least 10, 15, 20, 25, 30, 40, 50, 75, or 100 nucleotides or more, or the full length of the nucleic acid.

### DESCRIPTIVE EMBODIMENTS

The disclosure further provides diagnostic, prognostic and therapeutic methods, which are based, at least in part, on determination of the identify of a genotype of interest identified herein.

For example, information obtained using the diagnostic assays described herein is useful for determining if a subject is suitable for cancer treatment of a given type. Based on the prognostic information, a doctor can recommend a therapeutic protocol, useful for reducing the malignant mass or tumor in the patient or treat cancer in the individual.

A patient's likely clinical outcome following a clinical procedure such as a therapy or surgery can be expressed in relative terms. For example, a patient having a particular genotype or expression level can experience relatively longer overall survival than a patient or patients not having the genotype or expression level. The patient having the particular genotype or expression level, alternatively, can be considered as likely to survive. Similarly, a patient having a particular genotype or expression level can experience relatively longer progression free survival, or time to tumor progression, than a patient or patients not having the genotype or expression level. The patient having the particular genotype or expression level, alternatively, can be considered as not likely to suffer tumor progression. Further, a patient having a particular genotype or expression level can experience relatively shorter time to tumor recurrence than a patient or patients not having the genotype or expression level. The patient having the particular genotype or expression level, alternatively, can be considered as not likely to suffer tumor recurrence. Yet in another example, a patient having a particular genotype or expression level can experience relatively more complete response or partial response than a patient or patients not having the genotype or expression level. The patient having the particular genotype or expression level, alternatively, can be considered as likely to respond. Accordingly, a patient that is likely to survive, or not likely to suffer tumor progression, or not likely to suffer tumor recurrence, or likely to respond following a clinical procedure is considered suitable for the clinical procedure.

It is to be understood that information obtained using the diagnostic assays described herein can be used alone or in combination with other information, such as, but not limited to, genotypes or expression levels of other genes, clinical chemical parameters, histopathological parameters, or age, gender and weight of the subject. When used alone, the information obtained using the diagnostic assays described herein is useful in determining or identifying the clinical outcome of a treatment, selecting a patient for a treatment, or treating a patient, etc. When used in combination with other information, on the other hand, the information obtained using the diagnostic assays described herein is useful in aiding in the determination or identification of clinical outcome of a treatment, aiding in the selection of a patient for a treatment, or aiding in the treatment of a patient and etc. In a particular aspect, the genotypes or expression levels of one or more genes as disclosed herein are used in a panel of genes, each of which contributes to the final diagnosis, prognosis or treatment.

The methods are useful in the assistance of an animal, a mammal or yet further a human patient. For the purpose of illustration only, a mammal includes but is not limited to a human, a simian, a murine, a bovine, an equine, a porcine or an ovine subject.

### Diagnostic Methods

Provided, in one embodiment, is a method for selecting a colorectal cancer patient for a therapy comprising, or consisting essentially of, or yet further consisting of irinotecan and bevacizumab, comprising, or consisting essentially of, or yet further consisting of screening a biological sample isolated from the patient for an rs1792689, rs2268753, and/or rs17776182 polymorphism, and selecting the patient for the therapy if the genotype of (G/G) for rs1792689, (C/T) or (C/C) for rs2268753, or (G/G) for rs17776182 is present in the sample. In some aspects, the patient is not selected for the therapy if the genotype of (G/G) for rs1792689, (C/T) or (C/C) for rs2268753, or (G/G) for rs17776182 is not present in the sample. In some aspects, the patient is not selected for the therapy if the genotype of (A/G) or (A/A) for rs1792689, (T/T) for rs2268753, or (A/G) or (A/A) for rs17776182 is present in the sample. In some embodiments, the patient is selected for an irinotecan-free and/or bevacizumab-free therapy if the genotype of (G/G) for rs1792689, (C/T) or (C/C) for rs2268753, or (G/G) for rs17776182 is not present in the sample. In some embodiments, the patient is selected for an irinotecan-free and/or bevacizumab-free therapy if the genotype of (A/G) or (A/A) for rs1792689, (T/T) for rs2268753, or (A/G) or (A/A) for rs17776182 is present in the sample.

Provided in one embodiment, is a method for selecting a colorectal cancer patient for a therapy comprising, or consisting essentially of, or yet further consisting of irinotecan and bevacizumab, comprising, or consisting essentially of, or yet further consisting of screening a biological sample isolated from the patient for an rs7570532 and/or rs4946935 polymorphism, and selecting the patient for the therapy if the genotype of (A/A) for rs7570532 or (A/G) or (G/G) for rs4946935 is present in the sample. In some aspects, the patient is not selected for the therapy if the genotype (A/A) for rs7570532 or (A/G) or (G/G) for rs4946935 is not present in the sample. In some embodiments, the patient is not selected for therapy if the genotype of (A/G) for rs7570532 or (A/A) for rs4946935 is present in the sample. In some embodiments, the patient is selected for an irinotecan-free and/or bevacizumab-free therapy if the genotype of (A/A) for rs7570532 or (A/G) or (G/G) for rs4946935 is not present in the sample. In some embodiments, the patient is selected for an irinotecan-free and/or bevacizumab-free therapy if the genotype of (A/G) for rs7570532 or (A/A) for rs4946935 is present in the sample.

Provided in one embodiment, is a method for classifying a colorectal cancer patient as eligible for a therapy comprising, or consisting essentially of, or yet further consisting of irinotecan and bevacizumab, comprising, or consisting essentially of, or yet further consisting of screening a biological sample isolated from the patient for an rs7570532 and/or rs4946935 polymorphism, and classifying the patient as eligible for the therapy if the genotype of (A/A) for rs7570532 or (A/G) or (G/G) for rs4946935 is present in the sample.

Also provided is a method for identifying whether a colorectal cancer patient is likely to experience a relatively longer or shorter progression free survival following a therapy comprising, or consisting essentially of, or yet further consisting of irinotecan and bevacizumab, comprising, or consisting essentially of, or yet further consisting of screening a biological sample isolated from the patient for a rs1792689, rs2268753, and/or rs17776182 polymorphism, and identifying that the patient is likely to experience a longer progression free survival if genotype of (G/G) for rs1792689, (C/T) or (C/C) for rs2268753, or (G/G) for rs17776182 is present in the sample, relative to a colorectal cancer patient not having the genotype.

In one embodiment, provided is a method for identifying whether a colorectal cancer patient is likely to experience a relatively longer or shorter overall survival following a therapy comprising, or consisting essentially of, or yet further consisting of irinotecan and bevacizumab, comprising, or consisting essentially of, or yet further consisting of screening a biological sample isolated from the patient for an rs7570532 and/or rs4946935 polymorphism, and identifying that the patient is likely to experience a longer overall survival if the genotype of (A/A) for rs7570532 or (A/G) or (G/G) for rs4946935 is present in the sample, relative to a colorectal cancer patient not having the genotype.

In some aspects, therapy comprising, or consisting essentially of, or yet further consisting of irinotecan and bevacizumab further comprises a therapeutically effective amount of folinic acid and/or a pyrimidine analog. In some aspects, the therapy comprises FOLFIRI (leucovorin+Fluorouracil (5-FU)+irinotecan). In some aspects, the therapy further comprises therapeutically effective amounts of oxaliplatin. In some aspects, the therapy comprises FOLFOXFIRI (leucovorin+Fluorouracil (5-FU)+oxaliplatin+irinotecan).

In some aspects, the patient suffers from non-metastatic colorectal cancer or metastatic colorectal cancer. In some aspects, the colorectal cancer is metastatic or non-metastatic colon cancer. In some aspects, the colorectal cancer is metastatic or non-metastatic rectal cancer.

Any suitable method for identifying the genotype in the patient sample can be used and the disclosures described herein are not to be limited to these methods. For the purpose of illustration only, the genotype is determined by a method comprising, or consisting essentially of, or yet further consisting of, or alternatively consisting essentially of, or yet further consisting of, sequencing, hybridization, nucleic acid amplification, including polymerase chain reaction (PCR), real-time PCR, reverse transcriptase PCR (RT-PCR), nested PCR, ligase chain reaction, or PCR-RFLP, or microarray. These methods as well as equivalents or alternatives thereto are described herein.

The methods are useful in the assistance of an animal, a mammal or yet further a human patient. For the purpose of illustration only, a mammal includes but is not limited to a human, a simian, a murine, a bovine, an equine, a porcine or an ovine subject.

Information obtained using the diagnostic assays described herein is useful for determining if a subject will likely, more likely, or less likely to respond to cancer treatment of a given type. Based on the prognostic information, a doctor can recommend a therapeutic protocol, useful for treating reducing the malignant mass or tumor in the patient or treat cancer in the individual.

In addition, knowledge of the identity of a particular allele in an individual (the gene profile) allows customization of therapy for a particular disease to the individual's genetic profile, the goal of “pharmacogenomics”. For example, an individual's genetic profile can enable a doctor: 1) to more effectively prescribe a drug that will address the molecular basis of the disease or condition; 2) to better determine the appropriate dosage of a particular drug and 3) to identify novel targets for drug development. The identity of the genotype or expression patterns of individual patients can then be compared to the genotype or expression profile of the disease to determine the appropriate drug and dose to administer to the patient.

The ability to target populations expected to show the highest clinical benefit, based on the normal or disease genetic profile, can enable: 1) the repositioning of marketed drugs with disappointing market results; 2) the rescue of drug candidates whose clinical development has been discontinued as a result of safety or efficacy limitations, which are patient subgroup-specific; and 3) an accelerated and less costly development for drug candidates and more optimal drug labeling.

### Biological Sample Collection and Preparation

The methods and compositions disclosed herein can be used to detect nucleic acids associated with a rs1792689, rs2268753, rs17776182, rs7570532 and/or rs4946935 polymorphism using a biological sample obtained from a patient. Biological samples can be obtained by standard procedures and can be used immediately or stored, under conditions appropriate for the type of biological sample, for later use. Any liquid or solid biological material obtained from the patient believed to contain nucleic acids comprising, or consisting essentially of, or yet further consisting of the region containing the rs1792689, rs2268753, rs17776182, rs7570532 and/or rs4946935 polymorphism can be an suitable sample.

Methods of obtaining test samples are known to those of skill in the art and include, but are not limited to, aspirations, tissue sections, swabs, drawing of blood or other fluids, surgical or needle biopsies.

In some aspects, the biological sample is a tissue or a cell sample. Suitable patient samples in the methods include, but are not limited to, blood, plasma, serum, a biopsy tissue, fine needle biopsy sample, amniotic fluid, plasma, pleural fluid, saliva, semen, serum, tissue or tissue homogenates, frozen or paraffin sections of tissue or combinations thereof. In some aspects, the biological sample comprises, or alternatively consisting essentially of, or yet further consisting of, at least one of a tumor cell, a normal cell adjacent to a tumor, a normal cell corresponding to the tumor tissue type, a blood cell, a peripheral blood lymphocyte, or combinations thereof. In some aspects, the biological sample is an original sample recently isolated from the patient, a fixed tissue, a frozen tissue, a resection tissue, or a micro dissected tissue. In some aspects, the biological samples are processed, such as by sectioning of tissues, fractionation, purification, nucleic acid isolation, or cellular organelle separation.

In some embodiments, nucleic acid (DNA or RNA) is isolated from the sample according to any methods known to those of skill in the art. In some aspects, genomic DNA is isolated from the biological sample. In some aspects, RNA is isolated from the biological sample. In some aspects, cDNA is generated from mRNA in the sample. In some embodiments, the nucleic acid is not isolated from the biological sample (e.g., the polymorphism is detected directly from the biological sample).

### Detection of Polymorphisms

In some aspects, detection of polymorphisms can be accomplished by molecular cloning of the specified allele and subsequent sequencing of that allele using techniques known in the art, in some aspects, after isolation of a suitable nucleic acid sample. In some aspects, the gene sequences can be amplified directly from a genomic DNA preparation from the biological sample using PCR, and the sequence composition is determined by sequencing the amplified product (i.e., amplicon). Alternatively, the PCR product can be analyzed following digestion with a restriction enzyme, a method known as PCR-RFLP.

In some embodiments, the polymorphism is detected using allele specific hybridization using probes overlapping the polymorphic site. In some aspects, the nucleic acid probes are between 5 and 40 nucleotides in length. In some aspects, the nucleic acid probes are about 5, about 10, about 15, about 20, about 25, about 30, about 35, or about 40 or more nucleotides flanking the polymorphic site. For example, in some embodiments, the nucleic acid specifically binds to a nucleic acid having the sequence of any of SEQ ID NO: 1-3, 17 and 18 and overlaps the polymorphic site. Exemplary probes include nucleic acid probes having about 5, about 10, about 15, about 20, about 25, about 30, about 35, about 40 or more contiguous nucleotides of any of SEQ ID NO: 1-3, 17 and 18 and overlaps the polymorphic site.

In another embodiment of the disclosure, several nucleic acid probes capable of hybridizing specifically to the nucleic acid containing the allelic variant are attached to a solid phase support, e.g., a “chip” or “microarray. Such gene chips or microarrays can be used to detect genetic variations by a number of techniques known to one of skill in the art. In one technique, oligonucleotides are arrayed on a gene chip for determining the DNA sequence by the sequencing by hybridization approach. The probes of the disclosure also can be used for fluorescent detection of a genetic sequence. A probe also can be affixed to an electrode surface for the electrochemical detection of nucleic acid sequences.

In one aspect, “gene chips” or “microarrays” containing probes or primers for the gene of interest are provided alone or in combination with other probes and/or primers. A suitable sample is obtained from the patient extraction of genomic DNA, RNA, or any combination thereof and amplified if necessary. The DNA or RNA sample is contacted to the gene chip or microarray panel under conditions suitable for hybridization of the gene(s) of interest to the probe(s) or primer(s) contained on the gene chip or microarray. The probes or primers can be detectably labeled thereby identifying the polymorphism in the gene(s) of interest. Alternatively, a chemical or biological reaction can be used to identify the probes or primers which hybridized with the DNA or RNA of the gene(s) of interest. The genetic profile of the patient is then determined with the aid of the aforementioned apparatus and methods.

In some aspects, whole genome sequencing, in particular with the “next generation sequencing” techniques, which employ massively parallel sequencing of DNA templates, can be used to obtain genotypes of relevant polymorphisms. Exemplary NGS sequencing platforms for the generation of nucleic acid sequence data include, but are not limited to, Illumina's sequencing by synthesis technology (e.g., Illumina MiSeq or HiSeq System), Life Technologies' Ion Torrent semiconductor sequencing technology (e.g., Ion Torrent PGM or Proton system), the Roche (454 Life Sciences) GS series and Qiagen (Intelligent BioSystems) Gene Reader sequencing platforms.

In some aspects, nucleic acid comprising, or consisting essentially of, or yet further consisting of the polymorphism is amplified to produce an amplicon containing the polymorphism. Nucleic acids can be amplified by various methods known to the skilled artisan. Nucleic acid amplification can be linear or exponential. Amplification is generally carried out using polymerase chain reaction (PCR) technologies. Alternative or modified PCR amplification methods can also be used and include, for example, isothermal amplification methods, rolling circle methods, Hot-start PCR, real-time PCR, Allele-specific PCR, Assembly PCR or Polymerase Cycling Assembly (PCA), Asymmetric PCR, Colony PCR, Emulsion PCR, Fast PCR, Real-Time PCR, nucleic acid ligation, Gap Ligation Chain Reaction (Gap LCR), Ligation-mediated PCR, Multiplex Ligation-dependent Probe Amplification, (MLPA), Gap Extension Ligation PCR (GEXL-PCR), quantitative PCR (Q-PCR), Quantitative real-time PCR (QRT-PCR), multiplex PCR, Helicase-dependent amplification, Intersequence-specific (ISSR) PCR, Inverse PCR, Linear-After-The-Exponential-PCR (LATE-PCR), Methylation-specific PCR (MSP), Nested PCR, Overlap-extension PCR, PAN-AC assay, Reverse Transcription PCR(RT-PCR), Rapid Amplification of cDNA Ends (RACE PCR), Single molecule amplification PCR(SMA PCR), Thermal asymmetric interlaced PCR (TAIL-PCR), Touchdown PCR, long PCR, nucleic acid sequencing (including DNA sequencing and RNA sequencing), transcription, reverse transcription, duplication, DNA or RNA ligation, and other nucleic acid extension reactions known in the art. The skilled artisan will understand that other methods can be used either in place of, or together with, PCR methods, including enzymatic replication reactions developed in the future. See, e.g., Saiki, “Amplification of Genomic DNA” in PCR Protocols, Innis et al., eds., Academic Press, San Diego, Calif., 13-20 (1990); Wharam, et al., 29(11) Nucleic Acids Res, E54-E54 (2001); Hafner, et al., 30(4) Biotechniques, 852-6, 858, 860 passim (2001).

In some aspects, nucleic acid comprising, or consisting essentially of, or yet further consisting of the rs1792689, rs2268753, and/or rs17776182 polymorphism is amplified to produce an amplicon containing the rs1792689, rs2268753, and/or rs17776182 polymorphism. For example, in some aspects, nucleic acid comprising, or consisting essentially of, or yet further consisting of SEQ ID NO: 1, 2, or 3 is amplified to generate an amplicon comprising, or consisting essentially of, or yet further consisting of any of SEQ ID NO: 1, 2, or 3, respectively (e.g. an amplicon having the sequence of SEQ ID NO: 10, 11, or 12, respectively). In some aspects, nucleic acid containing the rs1792689, rs2268753, and/or rs17776182 polymorphism is amplified using a forward primer and a reverse primer the flank the rs1792689, rs2268753, and/or rs17776182 polymorphism. For example, nucleic acid containing the rs1792689 polymorphism is amplified using a forward primer comprising, or consisting essentially of, or yet further consisting of nucleic acid having the sequence of SEQ ID NO: 4 and a reverse primer comprising, or consisting essentially of, or yet further consisting of nucleic acid having the sequence of SEQ ID NO: 5, nucleic acid containing the rs2268753 polymorphism is amplified using a forward primer comprising, or consisting essentially of, or yet further consisting of nucleic acid having the sequence of SEQ ID NO: 6 and a reverse primer comprising, or consisting essentially of, or yet further consisting of nucleic acid having the sequence of SEQ ID NO: 7, and/or nucleic acid containing the rs17776182 polymorphism is amplified using a forward primer comprising, or consisting essentially of, or yet further consisting of nucleic acid having the sequence of SEQ ID NO: 8 and a reverse primer comprising, or consisting essentially of, or yet further consisting of nucleic acid having the sequence of SEQ ID NO: 9. In some aspects, the amplicon containing the rs1792689, rs2268753, and/or rs17776182 polymorphism is detected using a nucleic acid probe. In some aspects, the amplicon containing the rs1792689, rs2268753, and/or rs17776182 polymorphism (e.g. an amplicon having the sequence of SEQ ID NO: 10, 11, or 12, respectively) is detected by hybridizing a nucleic acid probe containing the rs1792689, rs2268753, and/or rs17776182 polymorphism or a complement thereof to the corresponding complementary strand of the amplicon and detecting the hybrid formed between the nucleic acid probe and the complementary strand of the amplicon. In some aspects, amplicon containing the rs1792689, rs2268753, and/or rs17776182 polymorphism is sequenced (e.g., dideoxy chain termination methods (Sanger method and variants thereof), Maxam & Gilbert sequencing, pyrosequencing, exonuclease digestion and next-generation sequencing methods).

In some aspects, nucleic acid comprising, or consisting essentially of, or yet further consisting of the rs7570532 and/or rs4946935 polymorphism is amplified to produce an amplicon containing the rs7570532 and/or rs4946935 polymorphism. For example, in some aspects, nucleic acid comprising, or consisting essentially of, or yet further consisting of SEQ ID NO: 17 and/or 18 is amplified to generate an amplicon comprising, or consisting essentially of, or yet further consisting of any of SEQ ID NO: 17 or 18, respectively. In some aspects, nucleic acid containing the rs7570532 and/or rs4946935 polymorphism is amplified using a forward primer and a reverse primer the flank the rs7570532 and/or rs4946935. For example, nucleic acid containing the rs7570532 polymorphism is amplified using a forward primer comprising, or consisting essentially of, or yet further consisting of nucleic acid having the sequence of SEQ ID NO: 13 and a reverse primer comprising, or consisting essentially of, or yet further consisting of nucleic acid having the sequence of SEQ ID NO: 14, and/or nucleic acid containing the rs4946935 polymorphism is amplified using a forward primer comprising, or consisting essentially of, or yet further consisting of nucleic acid having the sequence of SEQ ID NO: 15 and a reverse primer comprising, or consisting essentially of, or yet further consisting of nucleic acid having the sequence of SEQ ID NO: 16. In some aspects, the amplicon containing the rs7570532 and/or rs4946935 polymorphism is detected using a nucleic acid probe. In some aspects, the amplicon containing the rs7570532 and/or rs4946935 polymorphism is detected by hybridizing a nucleic acid probe containing the rs7570532 and/or rs4946935 polymorphism or a complement thereof to the corresponding complementary strand of the amplicon and detecting the hybrid formed between the nucleic acid probe and the complementary strand of the amplicon. In some aspects, amplicon containing the rs7570532 and/or rs4946935 polymorphism is sequenced (e.g., dideoxy chain termination methods (Sanger method and variants thereof), Maxam & Gilbert sequencing, pyrosequencing, exonuclease digestion and next-generation sequencing methods).

In some embodiments, the amplification includes a labeled primer or probe, thereby allowing detection of the amplification products corresponding to that primer or probe. In particular embodiments, the amplification can include a multiplicity of labeled primers or probes; such primers can be distinguishably labeled, allowing the simultaneous detection of multiple amplification products.

In some embodiments, the amplification products are detected by any of a number of methods such as gel electrophoresis, column chromatography, hybridization with a nucleic acid probe, or sequencing the amplicon.

Detectable labels can be used to identify the primer or probe hybridized to a genomic nucleic acid or amplicon. Detectable labels include but are not limited to fluorophores, isotopes (e.g., ³²P, ³³P, ³⁵S, ³H, ¹⁴C, ¹²⁵I, ¹³¹I) electron-dense reagents (e.g., gold, silver), nanoparticles, enzymes commonly used in an ELISA (e.g., horseradish peroxidase, beta-galactosidase, luciferase, alkaline phosphatase), chemiluminescent compounds, colorimetric labels (e.g., colloidal gold), magnetic labels (e.g., Dynabeads®), biotin, digoxigenin, haptens, proteins for which antisera or monoclonal antibodies are available, ligands, hormones, oligonucleotides capable of forming a complex with the corresponding oligonucleotide complement.

In one embodiment, a primer or probe is labeled with a fluorophore that emits a detectable signal. The term “fluorophore” as used herein refers to a molecule that absorbs light at a particular wavelength (excitation frequency) and subsequently emits light of a longer wavelength (emission frequency). While a suitable reporter dye is a fluorescent dye, any reporter dye that can be attached to a detection reagent such as an oligonucleotide probe or primer is suitable for use in the methods described. Suitable fluorescent moieties include, but are not limited to, the following fluorophores working individually or in combination: 4-acetamido-4′-isothiocyanatostilbene-2,2′disulfonic acid; acridine and derivatives, e.g. acridine, acridine isothiocyanate; Alexa Fluors: Alexa Fluor® 350, Alexa Fluor® 488, Alexa Fluor® 546, Alexa Fluor® 555, Alexa Fluor® 568, Alexa Fluor® 594, Alexa Fluor® 647 (Molecular Probes); 5-(2′-aminoethyl)aminonaphthalene-1-sulfonic acid (EDANS); 4-amino-N-[3-vinylsulfonyl)phenyl]naphthalimide-3,5 disulfonate (Lucifer Yellow VS); N-(4-anilino-1-naphthyl)maleimide; anthranilamide; Black Hole Quencher™ (BHQ™) dyes (biosearch Technologies); BODIPY dyes: BODIPY® R-6G, BOPIPY® 530/550, BODIPY® FL; Brilliant Yellow; coumarin and derivatives: coumarin, 7-amino-4-methylcoumarin (AMC, Coumarin 120), 7-amino-4-trifluoromethylcouluarin (Coumarin 151); Cy2®, Cy3®, Cy3.5®, Cy5®, Cy5.5®; cyanosine; 4′,6-diaminidino-2-phenylindole (DAPI); 5′,5″-dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); Eclipse™ (Epoch Biosciences Inc.); eosin and derivatives: eosin, eosin isothiocyanate; erythrosin and derivatives: erythrosin B, erythrosin isothiocyanate; ethidium; fluorescein and derivatives: 5-carboxyfluorescein (FAM), 5-(4,6-dichlorotriazin-2-yl)aminofluorescein (DTAF), 2′,7′-dimethoxy-4′5′-dichloro-6-carboxyfluorescein (JOE), fluorescein, fluorescein isothiocyanate (FITC), hexachloro-6-carboxyfluorescein (HEX), QFITC (XRITC), tetrachlorofluorescein (TET); fluorescamine; IR144; IR1446; lanthamide phosphors; Malachite Green isothiocyanate; 4-methylumbelliferone; ortho cresolphthalein; nitrotyrosine; pararosaniline; Phenol Red; B-phycoerythrin, R-phycoerythrin; allophycocyanin; o-phthaldialdehyde; Oregon Green®; propidium iodide; pyrene and derivatives: pyrene, pyrene butyrate, succinimidyl 1-pyrene butyrate; QSY® 7; QSY® 9; QSY® 21; QSY® 35 (Molecular Probes); Reactive Red 4 (Cibacron® Brilliant Red 3B-A); rhodamine and derivatives: 6-carboxy-X-rhodamine (ROX), 6-carboxyrhodamine (R6G), lissamine rhodamine B sulfonyl chloride, rhodamine (Rhod), rhodamine B, rhodamine 123, rhodamine green, rhodamine X isothiocyanate, riboflavin, rosolic acid, sulforhodamine B, sulforhodamine 101, sulfonyl chloride derivative of sulforhodamine 101 (Texas Red); terbium chelate derivatives; N,N,N′,N′-tetramethyl-6-carboxyrhodamine (TAMRA); tetramethyl rhodamine; and tetramethyl rhodamine isothiocyanate (TRITC).

In some aspects, the primer or probe is further labeled with a quencher dye such as Tamra, Dabcyl, or Black Hole Quencher®(BHQ), especially when the reagent is used as a self-quenching probe such as a TaqMan® (U.S. Pat. Nos. 5,210,015 and 5,538,848) or Molecular Beacon probe (U.S. Pat. Nos. 5,118,801 and 5,312,728), or other stemless or linear beacon probe (Livak et al., 1995, PCR Method Appl., 4:357-362; Tyagi et al, 1996, Nature Biotechnology, 14:303-308; Nazarenko et al., 1997, Nucl. Acids Res., 25:2516-2521; U.S. Pat. Nos. 5,866,336 and 6,117,635).

In some aspects, methods for real time PCR use fluorescent primers/probes, such as the TaqMan® primers/probes (Heid, et al., Genome Res 6: 986-994, 1996), molecular beacons, and Scorpion™ primers/probes. Real-time PCR quantifies the initial amount of the template with more specificity, sensitivity and reproducibility, than other forms of quantitative PCR, which detect the amount of final amplified product. Real-time PCR does not detect the size of the amplicon. The probes employed in Scorpion®™ and TaqMan® technologies are based on the principle of fluorescence quenching and involve a donor fluorophore and a quenching moiety. The term “donor fluorophore” as used herein means a fluorophore that, when in close proximity to a quencher moiety, donates or transfers emission energy to the quencher. As a result of donating energy to the quencher moiety, the donor fluorophore will itself emit less light at a particular emission frequency that it would have in the absence of a closely positioned quencher moiety. The term “quencher moiety” as used herein means a molecule that, in close proximity to a donor fluorophore, takes up emission energy generated by the donor and either dissipates the energy as heat or emits light of a longer wavelength than the emission wavelength of the donor. In the latter case, the quencher is considered to be an acceptor fluorophore. The quenching moiety can act via proximal (i.e., collisional) quenching or by Forster or fluorescence resonance energy transfer (“FRET”). Quenching by FRET is generally used in TaqMan® primers/probes while proximal quenching is used in molecular beacon and Scorpion™ type primers/probes.

The detectable label can be incorporated into, associated with or conjugated to a nucleic acid primer or probe. Labels can be attached by spacer arms of various lengths to reduce potential steric hindrance or impact on other useful or desired properties. See, e.g., Mansfield, Mol. Cell. Probes (1995), 9:145-156.

Detectable labels can be incorporated into nucleic acid probes by covalent or non-covalent means, e.g., by transcription, such as by random-primer labeling using Klenow polymerase, or nick translation, or, amplification, or equivalent as is known in the art. For example, a nucleotide base is conjugated to a detectable moiety, such as a fluorescent dye, e.g., Cy3™ or Cy5™ and then incorporated into nucleic acid probes during nucleic acid synthesis or amplification. Nucleic acid probes can thereby be labeled when synthesized using Cy3™- or Cy5™-dCTP conjugates mixed with unlabeled dCTP.

Nucleic acid probes can be labeled by using PCR or nick translation in the presence of labeled precursor nucleotides, for example, modified nucleotides synthesized by coupling allylamine-dUTP to the succinimidyl-ester derivatives of the fluorescent dyes or haptens (such as biotin or digoxigenin) can be used; this method allows custom preparation of most common fluorescent nucleotides, see, e.g., Henegariu et al., Nat. Biotechnol. (2000), 18:345-348.

Nucleic acid probes can be labeled by non-covalent means known in the art. For example, Kreatech Biotechnology's Universal Linkage System® (ULS®) provides a non-enzymatic labeling technology, wherein a platinum group forms a co-coordinative bond with DNA, RNA or nucleotides by binding to the N7 position of guanosine. This technology can also be used to label proteins by binding to nitrogen and sulfur containing side chains of amino acids. See, e.g., U.S. Pat. Nos. 5,580,990; 5,714,327; and 5,985,566; and European Patent No. 0539466.

Labeling with a detectable label also can include a nucleic acid attached to another biological molecule, such as a nucleic acid, e.g., an oligonucleotide, or a nucleic acid in the form of a stem-loop structure as a “molecular beacon” or an “aptamer beacon”. Molecular beacons as detectable moieties are described; for example, Sokol (Proc. Natl. Acad. Sci. USA (1998), 95:11538-11543) synthesized “molecular beacon” reporter oligodeoxynucleotides with matched fluorescent donor and acceptor chromophores on their 5′ and 3′ ends. In the absence of a complementary nucleic acid strand, the molecular beacon remains in a stem-loop conformation where fluorescence resonance energy transfer prevents signal emission. On hybridization with a complementary sequence, the stem-loop structure opens increasing the physical distance between the donor and acceptor moieties thereby reducing fluorescence resonance energy transfer and allowing a detectable signal to be emitted when the beacon is excited by light of the appropriate wavelength. See also, e.g., Antony (Biochemistry (2001), 40:9387-9395), describing a molecular beacon consist of a G-rich 18-mer triplex forming oligodeoxyribonucleotide. See also U.S. Pat. Nos. 6,277,581 and 6,235,504.

Aptamer beacons are similar to molecular beacons; see, e.g., Hamaguchi, Anal. Biochem. (2001), 294:126-131; Poddar, Mol. Cell. Probes (2001), 15:161-167; Kaboev, Nucleic Acids Res. (2000), 28:E94. Aptamer beacons can adopt two or more conformations, one of which allows ligand binding. A fluorescence-quenching pair is used to report changes in conformation induced by ligand binding. See also, e.g., Yamamoto et al., Genes Cells (2000), 5:389-396; Smimov et al., Biochemistry (2000), 39:1462-1468.

The nucleic acid primer or probe can be indirectly detectably labeled via a peptide. A peptide can be made detectable by incorporating predetermined polypeptide epitopes recognized by a secondary reporter (e.g., leucine zipper pair sequences, binding sites for secondary antibodies, transcriptional activator polypeptide, metal binding domains, epitope tags). A label can also be attached via a second peptide that interacts with the first peptide (e.g., S—S association).

As readily recognized by one of skill in the art, detection of the complex containing the nucleic acid from a sample hybridized to a labeled probe can be achieved through use of a labeled antibody against the label of the probe. In one example, the probe is labeled with digoxigenin and is detected with a fluorescent labeled anti-digoxigenin antibody. In another example, the probe is labeled with FITC, and detected with fluorescent labeled anti-FITC antibody. These antibodies are readily available commercially. In another example, the probe is labeled with FITC, and detected with anti-FITC antibody primary antibody and a labeled anti-anti FITC secondary antibody.

Nucleic acids can be amplified prior to detection or can be detected directly during an amplification step (i.e., “real-time” methods, such as in TaqMan® and Scorpion™ methods). In some embodiments, the target sequence is amplified using a labeled primer such that the resulting amplicon is detectably labeled. In some embodiments, the primer is fluorescently labeled. In some embodiments, the target sequence is amplified and the resulting amplicon is detected by electrophoresis.

With regard to the exemplary primers and probes, those skilled in the art will readily recognize that nucleic acid molecules can be double-stranded molecules and that reference to a particular site on one strand refers, as well, to the corresponding site on a complementary strand. In defining a variant position, allele, or nucleotide sequence, reference to an adenine, a thymine (uridine), a cytosine, or a guanine at a particular site on one strand of a nucleic acid molecule also defines the thymine (uridine), adenine, guanine, or cytosine (respectively) at the corresponding site on a complementary strand of the nucleic acid molecule. Thus, reference can be made to either strand in order to refer to a particular variant position, allele, or nucleotide sequence. Probes and primers, can be designed to hybridize to either strand and detection methods disclosed herein can generally target either strand.

In some embodiments, the primers and probes comprise additional nucleotides corresponding to sequences of universal primers (e.g., T7, M13, SP6, T3) which add the additional sequence to the amplicon during amplification to permit further amplification and/or prime the amplicon for sequencing.

### Methods of Treatment

The disclosure further provides methods of treating a patient selected by any method of the above embodiments, or identified as likely to experience a more favorable clinical outcome by any of the above methods, following the therapy. In some embodiments, the methods entail administering to the patients such a therapy.

In one aspect provided is a method for treating a colorectal cancer patient with a therapy comprising, or consisting essentially of, or yet further consisting of an effective amount of irinotecan and bevacizumab, wherein a sample isolated from the patient is characterized by a polymorphism of the group of (G/G) for rs1792689, (C/T) or (C/C) for rs2268753, (G/G) for rs17776182, (A/A) for rs7570532, and (A/G) or (G/G) for rs4946935.

In another aspect provided is a method for treating a colorectal cancer patient with a therapy excluding an effective amount of irinotecan and bevacizumab, wherein a sample isolated from the patient is characterized by a polymorphism of the group of (A/G) or (A/A) for rs1792689, (T/T) for rs2268753, (A/G) or (A/A) for rs17776182, (A/G) for rs7570532, and (A/A) for rs4946935.

In another aspect provided is a method for treating a colorectal cancer patient with an effective amount of a therapy comprising, or consisting essentially of, or yet further consisting of irinotecan and bevacizumab, the method comprising, or consisting essentially of, or yet further consisting of determining if the patient's sample comprises a polymorphism from the group of rs1792689, rs2268753, rs17776182, rs7570532 and rs4946935; and if the patient has (G/G) for rs1792689, (C/T) or (C/C) for rs2268753, (G/G) for rs17776182, (A/A) for rs7570532, and (A/G) or (G/G) for rs4946935, then administering an effective amount of the therapy.

In some embodiments, provided are methods for treating a colorectal cancer patient selected for treatment based on the presence of the genotype of (G/G) for rs1792689, (C/T) or (C/C) for rs2268753, or (G/G) for rs17776182 in a biological sample from the patient, comprising, or consisting essentially of, or yet further consisting of administering to the patient a therapy comprising, or consisting essentially of, or yet further consisting of a therapeutically effective amount of irinotecan and bevacizumab. In some embodiments, the patient is treated with an irinotecan-free and/or bevacizumab-free therapy if the genotype of (G/G) for rs1792689, (C/T) or (C/C) for rs2268753, or (G/G) for rs17776182 is not present in the sample. In some embodiments, the patient is treated with an irinotecan-free and/or bevacizumab-free therapy if the genotype of (A/G) or (A/A) for rs1792689, (T/T) for rs2268753, or (A/G) or (A/A) for rs17776182 is present in the sample.

In some aspects, the patient is selected by a method comprising, or consisting essentially of, or yet further consisting of screening a tissue or cell sample isolated from the patient for the rs1792689, rs2268753, and/or rs17776182 polymorphism. Exemplary methods for screening are described in the diagnostic methods provided above and throughout the present disclosure. Any such diagnostic methods disclosed for the detection of a rs1792689, rs2268753, and/or rs17776182 polymorphism can be combined with the treatment methods provided herein.

In one embodiment, provided is a method for treating a colorectal cancer patient selected for treatment based on the presence of the genotype of (A/A) for rs7570532 or (A/G) or (G/G) for rs4946935 in a biological sample from the patient, comprising, or consisting essentially of, or yet further consisting of administering to the patient a therapy comprising, or consisting essentially of, or yet further consisting of a therapeutically effective amount of irinotecan and bevacizumab, or an equivalent of each thereof. In some embodiments, the patient is treated with an irinotecan-free and/or bevacizumab-free therapy if the genotype of (A/A) for rs7570532 or (A/G) or (G/G) for rs4946935 is not present in the sample. In some embodiments, the patient is treated with an irinotecan-free and/or bevacizumab-free therapy if the genotype of (A/G) for rs7570532 or (A/A) for rs4946935 is present in the sample. In some embodiments the patient was selected by a method comprising, consisting essentially of, or yet further consisting of screening a biological sample isolated from the patient for the genotypes.

In some aspects, the patient is selected by a method comprising, or consisting essentially of, or yet further consisting of screening a tissue or cell sample isolated from the patient for the rs7570532 and/or rs4946935 polymorphism. Exemplary methods for screening are described in the diagnostic methods provided above and throughout the present disclosure. Any such diagnostic methods disclosed for the detection of a rs7570532 and/or rs4946935 polymorphism can be combined with the treatment methods provided herein.

In some aspects, therapy comprising, or consisting essentially of, or yet further consisting of irinotecan and bevacizumab further comprises a therapeutically effective amount of folinic acid and/or a pyrimidine analog. In some aspects, the therapy comprises FOLFIRI (leucovorin+Fluorouracil (5-FU)+irinotecan). In some aspects, the therapy further comprises a therapeutically effective amount of oxaliplatin. In some aspects, the therapy comprises FOLFOXFIRI (leucovorin+Fluorouracil (5-FU)+oxaliplatin+irinotecan). In some embodiments the thereapy comprising, or consisting essentially of, or yet further consisting of irinotecan and bevacizumab is a first-line therapy. In some embodiments, the thereapy comprising, or consisting essentially of, or yet further consisting of irinotecan and bevacizumab is therapy subsequent to a first-line therapy.

In some aspects, the patient suffers from non-metastatic colorectal cancer or metastatic colorectal cancer. In some aspects, the colorectal cancer is colon cancer. In some aspects, the colorectal cancer is rectal cancer. In some embodiments, the colorectal cancer patient is RAS wild-type. In some embodiments, the colorectal cancer patient is KRAS and BRAF wild-type. In some embodiments, the patient suffers from non-metastatic colorectal cancer or metastatic colorectal cancer. In some embodiments the patient is a metastatic colorectal cancer patient.

Exemplary dosing schedules for the treatment of colorectal cancer with bevacizumab include but are not limited to 5-10 mg/kg IV every two weeks.

Exemplary dosing schedules for the treatment of colorectal cancer with irinotecan include but are not limited to 125 mg/m² IV infusion over 90 minutes on days 1, 8, 15, 22, then 2 weeks off, then repeat or 350 mg/m² IV infusion over 30-90 minutes once every 3 weeks. Exemplary dosing schedules for the treatment of colorectal cancer with irinotecan as combination therapy include but are not limited to 180 mg/m² IV infusion over 30-90 minutes once on days 1, 15, and 29 IV (infuse over 30-90 min), followed by infusion with leucovorin and 5-fluorouracil; next cycle begins on day 43 (6 week cycle) or 125 mg/m² on days 1, 8, 15, and 22 (infuse over 90 min), followed by bolus doses of leucovorin and 5-fluorouracil.

The methods are useful in the assistance of an animal, a mammal or yet further a human patient. For the purpose of illustration only, a mammal includes but is not limited to a human, a simian, a murine, a bovine, an equine, a porcine or an ovine subject. Accordingly, a formulation comprising, or consisting essentially of, or yet further consisting of the necessary therapy or equivalent thereof is further provided herein. The formulation can further comprise one or more preservatives or stabilizers.

The agents or drugs can be administered as a composition. A “composition” typically intends a combination of the active agent and another carrier, e.g., compound or composition, inert (for example, a detectable agent or label) or active, such as an adjuvant, diluent, binder, stabilizer, buffers, salts, lipophilic solvents, preservative, adjuvant or the like and include pharmaceutically acceptable carriers. Carriers also include pharmaceutical excipients and additives proteins, peptides, amino acids, lipids, and carbohydrates.

Various delivery systems are known and can be used to administer a chemotherapeutic agent of the disclosure, e.g., encapsulation in liposomes, microparticles, microcapsules, expression by recombinant cells, receptor-mediated endocytosis. See e.g., Wu and Wu (1987) J. Biol. Chem. 262:4429-4432 for construction of a therapeutic nucleic acid as part of a retroviral or other vector, etc. Methods of delivery include but are not limited to intra-arterial, intra-muscular, intravenous, intranasal and oral routes. In a specific embodiment, it can be desirable to administer the pharmaceutical compositions of the disclosure locally to the area in need of treatment; this can be achieved by, for example, and not by way of limitation, local infusion during surgery, by injection or by means of a catheter.

The agents identified herein as effective for their intended purpose can be administered to subjects or individuals identified by the methods herein as suitable for the therapy. Therapeutic amounts can be empirically determined and will vary with the pathology being treated, the subject being treated and the efficacy and toxicity of the agent.

Also provided is a therapy or a medicament comprising, or consisting essentially of, or yet further consisting of an effective amount of a chemotherapeutic as described herein for treatment of a human cancer patient having the appropriate expression level of the gene of interest as identified in the experimental examples. Further provided is a therapy comprising, or consisting essentially of, or yet further consisting of a platinum drug, or alternatively a platinum drug therapy, for use in treating a human cancer patient having the appropriate expression level of the gene of interest as identified in the experimental examples.

Methods of administering pharmaceutical compositions are well known to those of ordinary skill in the art and include, but are not limited to, oral, microinjection, intravenous or parenteral administration. The compositions are intended for topical, oral, or local administration as well as intravenously, subcutaneously, or intramuscularly. Administration can be effected continuously or intermittently throughout the course of the treatment. Methods of determining the most effective means and dosage of administration are well known to those of skill in the art and will vary with the cancer being treated and the patient and the subject being treated. Single or multiple administrations can be carried out with the dose level and pattern being selected by the treating physician.

### Kits

Kits or panel for use in detecting the rs1792689, rs2268753, rs17776182, rs7570532 and/or rs4946935 polymorphism in patient biological samples are provided. In some embodiments, a kit comprises at least one reagent necessary to perform the assay. For example, the kit can comprise an enzyme, a buffer or any other necessary reagent (e.g. PCR reagents and buffers). For example, in some aspects, a kit contains, in an amount sufficient for at least one assay, any of the hybridization assay probes, amplification primers, and/or antibodies suitable for detection in a packaging material. In some embodiments, the kit or panel comprises primer and/or probes suitable for screening for the rs1792689, rs2268753, and/or rs17776182 polymorphism. In some embodiments, the kit or panel comprises primer and/or probes suitable for screening for the rs1792689, rs2268753, rs17776182, rs7570532 and/or rs4946935 polymorphism.

The various components of the kit can be provided in a variety of forms. For example, in some aspects, the required enzymes, the nucleotide triphosphates, the probes, primers, and/or antibodies are be provided as a lyophilized reagent. These lyophilized reagents can be pre-mixed before lyophilization so that when reconstituted they form a complete mixture with the proper ratio of each of the components ready for use in the assay. In addition, the kits can contain a reconstitution reagent for reconstituting the lyophilized reagents of the kit. In exemplary kits for amplifying target nucleic acid derived from a colorectal cancer patients, the enzymes, nucleotide triphosphates and required cofactors for the enzymes are provided as a single lyophilized reagent that, when reconstituted, forms a proper reagent for use in the present amplification methods.

In some aspects, the kit or panel is for determining the likely clinical outcome of a colorectal cancer patient receiving a therapy comprising, or consisting essentially of, or yet further consisting of irinotecan and bevacizumab. In some aspects, the kit or panel is for determining the eligibility of a colorectal cancer patient for receiving a therapy comprising, or consisting essentially of, or yet further consisting of irinotecan and bevacizumab.

Typically, the kits will also include instructions recorded in a tangible form (e.g., contained on paper or an electronic medium) for using the packaged probes, primers, and/or antibodies in a detection assay for determining the presence or amount of the rs1792689, rs2268753, rs17776182, rs7570532 and/or rs4946935 polymorphism in a test sample.

In some aspects, the kits further comprise a solid support for anchoring the nucleic acid of interest on the solid support. The target nucleic acid can be anchored to the solid support directly or indirectly through a capture probe anchored to the solid support and capable of hybridizing to the nucleic acid of interest. Examples of such solid support include but are not limited to beads, microparticles (for example, gold and other nano particles), microarray, microwells, multiwell plates. The solid surfaces can comprise a first member of a binding pair and the capture probe or the target nucleic acid can comprise a second member of the binding pair. Binding of the binding pair members will anchor the capture probe or the target nucleic acid to the solid surface. Examples of such binding pairs include but are not limited to biotin/streptavidin, hormone/receptor, ligand/receptor, and antigen/antibody.

In one aspect, the kit further comprises an effective amount of the therapy. In one aspect, the therapy comprises, or alternatively consists essentially of, or yet alternatively consisting of, administration of a therapeutically effective amount of irinotecan and bevacizumab. In some aspects, therapy comprising, or consisting essentially of, or yet further consisting of irinotecan further comprises a therapeutically effective amount of folinic acid and/or a pyrimidine analog. In some aspects, the therapy comprises FOLFIRI (leucovorin+Fluorouracil (5-FU)+irinotecan). In some aspects, the therapy further comprises therapeutically effective amount of oxaliplatin. In some aspects, the therapy comprises FOLFOXFIRI (leucovorin+Fluorouracil (5-FU)+oxaliplatin+irinotecan).

The kit can comprise at least one probe or primer which is capable of specifically hybridizing to the gene of interest and instructions for use. For example, in some aspects, the kits comprise at least one of the above described nucleic acids. Exemplary kits for amplifying at least a portion of the gene of interest comprise two primers. For example, in some embodiments, the kit comprises a forward primer and a reverse primer that flank the polymorphism. For example, a forward primer comprising, or consisting essentially of, or yet further consisting of nucleic acid having the sequence of SEQ ID NO: 4 and a reverse primer comprising, or consisting essentially of, or yet further consisting of nucleic acid having the sequence of SEQ ID NO: 5, a forward primer comprising, or consisting essentially of, or yet further consisting of nucleic acid having the sequence of SEQ ID NO: 6 and a reverse primer comprising, or consisting essentially of, or yet further consisting of nucleic acid having the sequence of SEQ ID NO: 7, and/or a forward primer comprising, or consisting essentially of, or yet further consisting of nucleic acid having the sequence of SEQ ID NO: 8 and a reverse primer comprising, or consisting essentially of, or yet further consisting of nucleic acid having the sequence of SEQ ID NO: 9.

In some embodiments, the kit comprises a forward primer and a reverse primer that flank the polymorphism. In some embodiments, the kit comprises, or consists essentially of, or yet further consists of a forward primer comprising, or consisting essentially of, or yet further consisting of nucleic acid having the sequence of SEQ ID NO: 13 and a reverse primer comprising, or consisting essentially of, or yet further consisting of nucleic acid having the sequence of SEQ ID NO: 14, and/or a forward primer comprising, or consisting essentially of, or yet further consisting of nucleic acid having the sequence of SEQ ID NO: 15 and a reverse primer comprising, or consisting essentially of, or yet further consisting of nucleic acid having the sequence of SEQ ID NO: 16.

In some embodiments, the kit further comprises a nucleic acid probe for the detection of the amplicon. In some embodiments, the nucleic acid probe has about 5, about 10, about 15, about 20, or about 25, or about 30, about 35, about 40 or more contiguous nucleotides of any of SEQ ID NO: 1-3, 17 and 18 and overlaps the polymorphic site. In some aspects, the nucleic acid primers and/or probes are lyophilized.

In some embodiments, at least one of the primers for amplification is capable of hybridizing to the allelic variant sequence. For example, in some embodiments, at least one of the primers for amplification has about 5, about 10, about 15, about 20, or about 25, or about 30, about 35, about 40 or more contiguous nucleotides of any of SEQ ID NO: 1-3, 17 and 18 and overlaps the polymorphic site. Such kits are suitable for detection of genotype by, for example, fluorescence detection, by electrochemical detection, or by other detection.

Oligonucleotides, whether used as probes or primers, contained in a kit can be detectably labeled. Labels can be detected either directly, for example for fluorescent labels, or indirectly. Indirect detection can include any detection method known to one of skill in the art, including biotin-avidin interactions, antibody binding and the like. Fluorescently labeled oligonucleotides also can contain a quenching molecule. Oligonucleotides can be bound to a surface. In one embodiment, the surface is silica or glass. In another embodiment, the surface is a metal electrode.

The test samples used in the diagnostic kits include cells, protein or membrane extracts of cells, or biological fluids such as sputum, blood, serum, plasma, or urine. The test samples can also be a tumor cell, a normal cell adjacent to a tumor, a normal cell corresponding to the tumor tissue type, a blood cell, a peripheral blood lymphocyte, or combinations thereof. The test sample used in the above-described method will vary based on the assay format, nature of the detection method and the tissues, cells or extracts used as the sample to be assayed. Methods for preparing protein extracts or membrane extracts of cells are known in the art and can be readily adapted in order to obtain a sample which is compatible with the system utilized.

The kits can include all or some of the positive controls, negative controls, reagents, primers, sequencing markers, probes and antibodies described herein for determining the subject's genotype in the polymorphic region of the gene of interest or target region.

As amenable, these suggested kit components can be packaged in a manner customary for use by those of skill in the art. For example, these suggested kit components can be provided in solution or as a liquid dispersion or the like.

Typical packaging materials would include solid matrices such as glass, plastic, paper, foil, micro-particles and the like, capable of holding within fixed limits hybridization assay probes, and/or amplification primers. Thus, for example, the packaging materials can include glass vials used to contain sub-milligram (e.g., picogram or nanogram) quantities of a contemplated probe, primer, or antibodies or they can be microtiter plate wells to which probes, primers, or antibodies have been operatively affixed, i.e., linked so as to be capable of participating in an amplification and/or detection methods.

The instructions will typically indicate the reagents and/or concentrations of reagents and at least one assay method parameter which might be, for example, the relative amounts of reagents to use per amount of sample. In addition, such specifics as maintenance, time periods, temperature, and buffer conditions can also be included.

The diagnostic systems contemplate kits having any of the hybridization assay probes, amplification primers, or antibodies described herein, whether provided individually or in one of the combinations described above, for use in determining the presence or amount of rs1792689, rs2268753, rs17776182, rs7570532 and/or rs4946935 polymorphism in a test sample.

The disclosure now being generally described, it will be more readily understood by reference to the following example which is included merely for purposes of illustration of certain aspects and embodiments of the present disclosure, and are not intended to limit the disclosure.

### EXPERIMENTAL EXAMPLES

### Example 1: Irinotecan and Bevacizumab in Patients with rs1792689, rs2268753 and/or rs17776182 SNPs

This example shows that functional significant single nucleotide polymorphisms in genes involved in the degradation pathway predict clinical outcomes of metastatic colorectal cancer treated with irinotecan and bevacizumab therapies.

Results of the example to follow are summarized as follows. In the training cohort, patients with the FOXO3 rs4946935 A/A variant had a significantly shorter OS than those with any G allele (20.1 vs. 24.8 months) in both univariate (HR 1.58, 95% CI 1.03-2.43, P=0.03) and multivariate (HR 1.55, 95% CI 1.01-2.39, P=0.05) analyses. Among patients with RAS mutant tumors, those with the ACVR2B rs2268753 C/C variant had the longest OS (41.9 months) compared to those with the T/C (26.3 months) or T/T genotypes (16.7 months), both in univariable (P=0.045) and multivariable (P=0.036) analysis. Similarly, patients with RAS mutant tumors in the validation cohort and the ACVR2B rs2268753 T/C genotype had a superior RR (70%) and PFS (9.7 months) compared to those with the T/T variant (RR 48%, PFS 9.2 months; multivariable HR 0.51, P=0.019). There were no significant associations between any of the SNPs and outcomes in the FIRE3-cetuximab control cohort.

Genetic variants within cancer cachexia pathways may be prognostic and predictive markers in mCRC patients treated with bevacizumab-based chemotherapy, in a RAS specific manner.

Cancer cachexia is a multifactorial syndrome that commonly affects mCRC patients leading to progressive functional impairment. Cachexia occurs in 70% of pts with advanced cancer. The Activin/TGFbeta/SMAD pathway, known as cachexia pathway, plays a critical role in development and progression of CRC. In this example, single nucleotide polymorphisms (SNPs) of genes involved in the cachexia pathway were evaluated for their ability to predict clinical outcomes in irinotecan and bevacizumab (BV) treated mCRC patients.

Genomic DNA was obtained from mCRC patients receiving irinotecan and bevacizumab plus FOLFIRI as first line treatment and analyzed by using PCR-based direct sequencing. Nine functional SNPs in 5 genes (INHBA, MSTN, ACVR2B, SMAD2 and FOXO3) were tested in a discovery cohort of 294 pts in FIRE3 trial (NCT00433927), then validated in 230 pts in TRIBE trial (NCT00719797).

In the FIRE3 BV arm, main characteristics were the following: male/female=194/100; median age=65; RASwildtype/mutant=198/83; median PFS=10.2 months; median OS=24.2 months, median follow-up time=40.8 months.

PCR and product sequencing were done using standard procedures. Uni- and multivariate analyses, adjusting for age, gender, rash and racial background, were carried out.

Example PCR primers used in the example are provided in the table below.

| | Forward primer | Reverse primer |
|---|---|---|
| SNP | (SEQ ID NO.) | (SEQ ID NO.) |
| SMAD2 | 5′GCCAGGATGGTCTC | 5′TGATCTTATTATTCACCA |
| rs1792689 | AATCTC3′ | TCATCCA3′ |
| | (4) | (5) |
| ACVR2B | GGAGCTCAGGGTAGTG | GGACCCTGCCTCAGGACTAT |
| rs2268753 | CAAA | (7) |
| | (6) | |
| INHBA | TGTGATAGCCACAGCC | TTCCAAACCTCAGTGGCTTC |
| rs17776182 | TCAA | (9) |
| | (8) | |
| MSTN | CATCAGCGGATGAATG | GCATAGCTTAGCTCGCACTT |
| rs7570532 | GATA | G |
| | (13) | (14) |
| FOXO3 | CTCAGTCCGGAAGTCT | AAAATGCTCTGAAGTTGAAA |
| rs4946935 | AGAACAG | AGC |
| | (15) | (16) |

The amplicon generated comprising, or consisting essentially of, or yet further consisting of SMAD2 rs1792689 has the following sequence (Y=C or T):

(SEQ ID NO: 10)
5′GCCAGGATGGTCTCAATCTCTTGACCTTGTGATCCGCCTGCCTCGGCC
TCCCAAAGTGCTGGGCTTACAGGTGTGAGCCACCACGCCTGGCCCTGGCC
TGATATTAATAGTATCTACATTCTCTCTCAGGTGTTCYATTTTGGATGAT
GGTGAATAATAAGATCA 3′

The amplicon generated comprising, or consisting essentially of, or yet further consisting of ACVR2B rs2268753 has the following sequence (Y=C or T):

(SEQ ID NO: 11)
5′GGGAGCTCAGGGTAGTGCAAATGAGAACCAAGGAGTATCGGTTCAGGA
GTTTAGATCCAYTCACGGATACTGACCTGTCACCATGGATTGGGATCTGG
AGGGTTGAGGACTGGGTCTGGATAATATTTTTGCTAGTGACTGTAGATAG
ACTCTAGATAGTCCTGAGGCAGGGTCC3′

The amplicon generated comprising, or consisting essentially of, or yet further consisting of INHBA rs17776182 has the following sequence (R=A or G):

(SEQ ID NO: 12)
5′TGTGATAGCCACAGCCTCAAGGCTGTTTTTAGATGAAGGTGGAAATAC
RATGAAGATGATGCTCTGTTAGTTATCATTGATCAAGCACTCATTTCTGC
TACGCATGGAGCAAAGTGCTTGATATATGTGTAATACCCTTTCACCCTTC
AAATAACAATTTGAAATAGGTTTTCTTATTATTGCTAATTACAGATGAAG
CCACTGAGGTTTGGAA3′

Among RASwildtype patients, SMAD2 rs1792689 A/(N=34) achieved a significantly worse PFS compared to G/G variant carriers (N=157) in the univariate (median PFS 9.2 vs 10.8 months respectively, HR=1.50 [95% CI 1.022.22], p=0.037) and in the multivariate analysis (HR=1.58 [95% CI 1.062.34], p=0.023).

Among RASmutant patients, ACVR2B rs2268753 T/T (N=30) showed a significant worse OS compared to C/variant carriers (N=46) in the univariate (median OS 8.8 vs 11.2 months respectively, HR=1.81 [95% CI 1.063.09], p=0.029) and in the multivariate analysis (HR=2.13 [95% CI 1.193.81], p=0.011).

Among female patients, INHBA rs17776182 A/(N=31) showed a significant worse PFS compared to G/G variant carriers (N=59) in the univariate (median PFS 8.8 vs 10.1 months respectively, HR=1.61 [95% CI 1.012.57], p=0.045) and in the multivariate analysis (HR=1.75 [95% CI 1.082.85], p=0.024).

This study demonstrated that variations in genes regulating cancer cachexia may affect prognosis of mCRC patients treated with BV based chemotherapy.

### Example 2: Irinotecan and Bevacizumab in Patients with rs1792689, rs2268753, rs17776182, rs7570532 and/or rs4946935 SNPs

The research described below is an expansion of the work described in Example 1. Scientific publications are referenced by an Arabic number, the full citation of which is found immediately preceding the claims, the disclosures of which are incorporated herein by reference to more fully describe the state of the art.

Cancer cachexia is characterized by a loss of skeletal muscle mass that cannot be fully reversed by conventional nutritional support and leads to progressive functional decline [1]. Approximately 80% of advanced cancer patients develop cachexia [2] and its associated intolerance to chemotherapy, impaired quality of life, and poor prognosis [3-5].

Studies investigating the metabolic alterations underlying cancer cachexia [6, 7] have revealed roles for cytokine activation and tumor-derived growth factors, the targets of which are skeletal muscle gene products (this is confusing not sure what you mean targets muscle gene products) [8]. Activin and myostatin mediate one of the most critical pathways regulating skeletal muscle degradation [9]. Activin and myostatin are TGFβ superfamily ligands which are increased in serum in patients with cancer cachexia [10, 11]. These ligands bind their respective type I (ACVR1B or 1C) and type II (ACVR2B) receptors and activate the transcriptional factors, SMAD2 or SMAD3, to promote skeletal muscle breakdown. Such metabolic alterations are potentially responsible for not only skeletal muscle wasting, but also cancer progression and resistance to chemotherapy [12].

In addition, accumulating evidence suggests that activin/myostatin signaling may regulate angiogenesis [13]. In myostatin knockout mice, expression of genes involved in angiogenesis is reduced in the tumor [14], and ALK5 overexpression has been shown to promote tumor angiogenesis [15].

Mutations within the RAS oncogene, which are present in 50% of metastatic colorectal cancer (mCRC) patients [16], have been associated with the development of cachexia [17]. Patients with mCRC harboring RAS mutations are generally treated with first-line bevacizumab-based chemotherapy, as anti-epidermal growth factor receptor agents have no clinical benefit [18]. Bevacizumab has been shown to benefit mCRC patients, independent of RAS mutation status [19].

The goal of this example was to test the prognostic and predictive significance of cachexia-related genetic variants in mCRC patients treated with bevacizumab-based chemotherapy and whether these associations are depended on ras mutational status. Associations between single nucleotide polymorphisms (SNPs) in genes regulating cachexia (INHBA, MSTN, ALK4 [ACVR1B], ALK5 [TGFBR1], ALK7 [ACVR1C], ACVR2B, SMAD2, FOXO3) and outcomes in mCRC patients undergoing first-line FOLFIRI plus bevacizumab in two phase III trials, and a control cohort receiving FOLFIRI plus cetuximab were examined as disclosed herein.

A total of 820 patients were included in this study. Patients with sufficient tissue for analysis treated with first-line FOLFIRI plus bevacizumab in the randomized, open-label, phase III FIRE-3 trial [20] served as the training cohort (N=296; 87% of 343 enrolled patients). Patients with sufficient blood for analysis treated with first-line FOLFIRI plus bevacizumab in the randomized, open-label, phase III TRIBE trial [21, 22] served as the validation cohort (N=228; 89% of 256 enrolled patients). Patients treated with first-line FOLFIRI plus cetuximab in FIRE-3 served as the control cohort (N=296; 84% of 353 enrolled patients). Eligible patients had histologically proven metastatic colorectal adenocarcinoma, measurable disease according to Response Evaluation Criteria in Solid Tumors (RECIST) v1.1, and received no prior systemic chemotherapy for metastatic disease. Standard inclusion and exclusion criteria were applied. For the expression analysis, pretreatment tumor tissue samples were obtained from consecutive 43 mCRC patients receiving bevacizumab plus oxaliplatin based chemotherapy were analyzed.

This example was conducted in adherence to the reporting recommendations for tumor marker prognostic studies (REMARK) [23]. The tissue analysis was approved by the University of Southern California (USC) Institutional Review Board of Medical Sciences and conducted at the USC/Norris Comprehensive Cancer Center, in accordance with the Declaration of Helsinki and Good Clinical Practice Guidelines.

Candidate SNPs in INHBA, MSTN, ALK4 (ACVR1B), ALK5 (TGFBR1), ALK7 (ACVR1C), ACVR2B, SMAD2, FOXO3 with a minor allele frequency >10% in Europeans, according to the ENSEMBL database, were selected for analyses. Among these candidate SNPs, 12 polymorphisms were selected whose biologic significance was reported in the literature, or which were considered potentially functional according to the F-SNP database [24]. The characteristics of the selected polymorphisms are shown in the table.

Genomic DNA was extracted from formalin-fixed paraffin-embedded (FFPE) specimens in patients enrolled in FIRE-3, and from blood in patients enrolled in TRIBE using the QIAamp DNeasy Kit (Qiagen, Valencia, Calif., USA), according to the manufacturer's instructions. The primers used for polymerase chain reaction analyses are listed in the table. DNA sequences were analyzed using the ABI Sequencing Scanner version 1.0 (Applied Biosystems, Waltham, Mass., USA). Investigators involved in SNP analyses were blinded to the patients' clinical data. Genotyping was successful in at least 90% of cases in each polymorphism analyzed.

### Expression Analysis

The expression level of two genes (ACVR2B and VEGF) was measured in colorectal tumor tissue by quantitative real-time reverse-transcription polymerase chain reaction (qRT-PCR). Total RNA was isolated from paraffin embedded samples using a RNeasy FFPE Kit (Qiagen) according to the manufacture's protocol. Total RNA was reverse-transcribed into cDNA using qScript cDNA Synthesis Kit (Bio-Rad #170-8891; Quanta Biosciences) according to the manufacturer's instructions. Quantitative real-time PCR was performed in triplicates with primers specific for ACVR2B, VEGF and β-actin using an Applied Biosystems 7500 PCR Detection System (Applied Biosystems, Inc.) with SYBR green. β-actin was used as the internal control and relative gene expression levels were calculated as ΔΔCT. The sequences of the primers used in this study are listed below.

Primers

| | Location of | | | | Forward/Reverse primer |
|---|---|---|---|---|---|
| Gene | polymorphism | Change | Function | MAF^{a} | (5′-3′) |
| INHBA | intron | | | | F: TGTGATAGCCACAGCCTCAA |
| rs17776182 | Chr 7, | G > A | Risk of | 0.16 | R: TTCCAAACCTCAGTGGCTTC |
| | 41718280 | | testicular | | |
| | | | germ line | | |
| | | | tumor | | |
| INHBA | intron | | | | F: |
| | | | | | AAGATTTCACTGTGCCTAGATATGG |
| rs2237432 | Chr 7, | T > C | Risk of | 0.22 | R: |
| | 41695436 | | fertility | | AAGGAGGAATAACCTTAAGACCTC |
| | | | | | A |
| MSTN | intron | | | | F: CATCAGCGGATGAATGGATA |
| rs7570532 | Chr 2, | A > G | Risk of | 0.36 | R: GCATAGCTTAGCTCGCACTTG |
| | 190058686 | | osteopor- | | |
| | | | otic | | |
| | | | fracture | | |
| ALK4 | 3′UTR | | | | F: CTGGTGGAAGTCTTGGGTGT |
| (ACVR1B) | | | | | |
| rs2854464 | Chr12, | A > G | Muscle | 0.37 | R: TCTGGGAGATGAAAGACAGATG |
| | 51995107 | | strength | | |
| ALK5 | intron | | | | F: CTTGCCTTACCATGGGAGAA |
| (TGFBR1) | | | | | |
| rs10760673 | Chr 9, | G > A | Protein | 0.27 | R: ATTTCCTCAGGGACACACCA |
| | 99116340 | | coding | | |
| ALK7 | | | Transcrip- | | F: CCAGAGCTCACCATGTATCCT |
| (ACVR1C) | | | tional | | |
| | | | regulation | | |
| rs13010956 | Chr 2, | T > C | Metabolic | 0.33 | R: TTATGATGTGACCGCCTCTG |
| | 157556030 | | syndrome | | |
| ACVR2B | intron | | Transcrip- | | F: GGAGCTCAGGGTAGTGCAAA |
| | | | tional | | |
| | | | regulation | | |
| rs2268753 | Chr 3, | T > C | Associ- | 0.43 | R: GGACCCTGCCTCAGGACTAT |
| | 38458698 | | ated with | | |
| | | | ovarian | | |
| | | | failure | | |
| ACVR2B | 3′UTR | | | | F: AAGACTGCCAGTGAGGGAAG |
| rs13072731 | Chr 3, | C > A | Transcrip- | 0.39 | R: GGCATTGTTGTGGATTTGTG |
| | 38491844 | | tional | | |
| | | | regulation | | |
| SMAD2 | 3′UTR | | | | F: TGGACACGATTATTCCGCAAAA |
| rs1792671 | Chr 18, | C > T | Tag SNP | 0.47 | R: TGACCTTGTGATCCGCCTG |
| | 47835823 | | | | |
| SMAD2 | intron | | | | F: GCCACAGAGAAAGGAAAACA |
| rs1792689 | Chr 18, | G > A | Risk of | 0.13 | R: TACCCTCCAAACAGTTAACA |
| | 47842216 | | rectal | | |
| | | | cancer | | |
| FOXO3 | intron | | | | F: TCAGTTGGGTTGGAATTGGT |
| rs12212067 | Chr 6, | G > T | Associ- | 0.14 | R: CCCTCTGCGTTAGATTCTGG |
| | 108659993 | | ated with | | |
| | | | Crohn | | |
| | | | disease | | |
| FOXO3 | 3′UTR | | | | F: CTCAGTCCGGAAGTCTAGAACAG |
| rs4946935 | Chr 6, | A > G | Increased | 0.48 | R: |
| | 108682118 | | life span | | AAAATGCTCTGAAGTTGAAAAGC |

Abbreviation: 3′ UTR, 3′-untranslated region.
^{a}Minor allele frequency (MAF), according to the Ensemble database (phase I of the 1000 Genomes Project) for Europeans

Statistical Analysis

The primary objective was to evaluate the associations between genetic polymorphisms with progression-free survival (PFS) and overall survival (OS), defined as the period from the date of trial registration to the first observation of progression or death, and to death, respectively. If events were not observed, the endpoints were censored at the time of last contact or follow-up. With 251 PFS events in the training cohort, there would be 80% power to detect minimum hazard ratios (HRs) ranged from 1.43 to 1.86 for SNPs with the minor allele frequencies from 5% to 45% using a dominant mode of inheritance and a two-sided 0.05 level log-rank test. The power would be greater than 63% in the validation cohort (PFS events=174) and >80% in the control cohort (PFS events=258) for the same SNPs with the same allele frequency using the same test and the same mode of inheritance. The secondary objective was to assess the associations between genetic variants with RECIST-defined response rate in each cohort.

χ² tests or the Kruskal-Wallis test was carried out to examine the differences in baseline patient characteristics among the three cohorts. Kaplan-Meier curves and log-rank tests were carried out in univariable analysis for the association between candidate SNPs and PFS and OS using co-dominant, dominant, or recessive genetic model whenever appropriate. The Cox proportional hazards regression model was fitted to re-evaluate the association between SNPs and outcomes, considering the imbalance in the distributions of baseline characteristics within each cohort. The baseline demographic and clinical characteristics that remained statistically significantly associated with endpoints in multivariable analyses were included in the final models. Fisher's exact test was applied to test the association between the SNPs and tumor response. ACVR2B and VEGF gene expression levels were compared using non-parametric test, Kruskal-Wallis. SAS 9.4 (SAS Institute, Cary, N.C.) was used to perform all analyses. All tests were two-sided at a significance level of 0.05.

### Results

Baseline patient and tumor characteristics are summarized in Table 1. Compared to FIRE-3 cohorts, TRIBE was comprised of patients with younger age, lower ECOG-PS, less primary tumor resection and adjuvant chemotherapy, and more RAS mutant status. The median PFS, OS, and follow-up times were 10.2, 24.2, and 40.8 months in the training cohort (FIRE3-bevacizumab cohort); 9.7, 26.1, and 49.3 months in the validation cohort (TRIBE cohort); 9.6, 27.1, and 40.6 months in the control cohort (FIRE3-cetuximab cohort), respectively. The allelic frequencies for all SNPs were within the probability limits of Hardy-Weinberg Equilibrium (P>0.05).

TABLE 1
Baseline clinical characteristics of patients
in training, validation and control cohorts.

| | Training cohort | | Validation cohort | | Control cohort | | |
|---|---|---|---|---|---|---|---|
| | (n = 296) | | (n = 228) | | (n = 296) | | P |
| Variables | N (%) | | N (%) | | N (%) | | value ^{a} |
| Gender | | | | | | | 0.20 |
| Male | 195 | (66) | 138 | (61) | 201 | (68) | |
| Female | 101 | (34) | 90 | (39) | 95 | (32) | |
| Age | | | | | | | <0.001 |
| Median (range) | 64.5 | (31-76) | 60 | (29-75) | 64 | (38-79) | |
| <65 | 148 | (50) | 154 | (68) | 151 | (51) | |
| ≥65 | 148 | (50) | 74 | (32) | 145 | (49) | |
| Performance status | | | | | | | <0.001 |
| ECOG 0 | 163 | (64) | 188 | (82) | 151 | (51) | |
| ECOG 1-2 | 133 | (13) | 39 | (17) | 145 | (49) | |
| Unknown ^{b} | 0 | (0) | 1 | (0) | 0 | (0) | |
| Tumor site | | | | | | | 0.17 |
| Right side | 74 | (25) | 57 | (25) | 58 | (20) | |
| Left side | 215 | (73) | 156 | (68) | 229 | (77) | |
| Unknown ^{b} | 7 | (2) | 15 | (7) | 9 | (3) | |
| Time to metastases | | | | | | | 0.10 |
| Synchronous | 185 | (63) | 188 | (82) | 184 | (62) | |
| Metachoronous | 63 | (21) | 40 | (18) | 57 | (19) | |
| Unknown ^{b} | 48 | (16) | 0 | (0) | 55 | (19) | |
| Liver only disease | | | | | | | 0.90 |
| Yes | 96 | (32) | 72 | (32) | 99 | (33) | |
| No | 200 | (68) | 156 | (68) | 197 | (67) | |
| Number of metastases | | | | | | | 0.98 |
| <2 | 107 | (36) | 99 | (43) | 104 | (35) | |
| ≥2 | 142 | (48) | 129 | (57) | 140 | (47) | |
| Unknown ^{b} | 47 | (16) | 0 | (0) | 52 | (18) | |
| Primary tumor resection | | | | | | | <0.001 |
| Performed | 256 | (86) | 144 | (63) | 249 | (84) | |
| Unperformed | 40 | (14) | 84 | (37) | 45 | (15) | |
| Unknown ^{b} | 0 | (0) | 0 | (0) | 2 | (1) | |
| Adjuvant chemotherapy | | | | | | | 0.030 |
| Yes | 54 | (18) | 28 | (12) | 62 | (21) | |
| No | 242 | (82) | 200 | (88) | 232 | (78) | |
| Unknown ^{b} | 0 | (0) | 0 | (0) | 2 | (1) | |
| RAS status | | | | | | | <0.001 |
| Wildtype | 200 | (68) | 55 | (24) | 192 | (65) | |
| Mutant | 83 | (28) | 116 | (51) | 90 | (30) | |
| Unknown ^{b} | 13 | (4) | 57 | (25) | 14 | (5) | |

Abbreviations: ECOG, Eastern Cooperative Oncology Group.
^{a} Based on the χ2 test or the Kruskal-Wallis when appropriate.
^{b} Not included in the test.

### Associations Between Cachexia SNPs and Outcomes in the Training and Validation Cohort

First, associations between SNPs within the INHBA, MSTN, ALK4 (ACVR1B), ALK5 (TGFBR1), ALK7 (ACVR1C), ACVR2B, SMAD2, FOXO3 genes and clinical outcomes were examined in the FIRE-3 training cohort. Among the examined SNPs, FOXO3 rs4946935 was significantly associated with survival. Patients with the FOXO3 rs4946935 A/A variant had a significantly shorter OS than those with any G allele (20.1 vs. 24.8 months) in both univariable (HR 1.58, 95% CI 1.03-2.43, P=0.034) and multivariable (HR 1.55, 95% CI 1.01-2.39, P=0.046) analysis (Table 2 and its Supplement). However, these observations were not confirmed in the validation cohort. There were no other significant associations between the other SNPs and RR, PFS, or OS in the training or validation cohorts.

TABLE 2
Association between cachexia-related gene polymorphisms and clinical outcomes
according to RAS mutant status in the training cohort (FIRE3-bevacizumab cohort).

| Gene rs | | | | | Median, | | | | | Median, | | | | |
|---|---|---|---|---|---|---|---|---|---|---|---|---|---|---|
| number | | Tumor response | | | months | Progression-free Survival | | | | months | Overall Survival | | | |
| Genotype | N | PR + CR | SD + PD | P value* | (95% CI) | HR (95% CI)† | P value* | HR (95% CI)‡ | P value* | (95% CI) | HR (95% CI)† | P value* | HR (95% CI)‡ | P value* |

RAS wild-type

| SMAD2 | | | | 0.80 | | | 0.036 | | 0.022 | | | 0.16 | | 0.061 |
|---|---|---|---|---|---|---|---|---|---|---|---|---|---|---|
| rs1792689 | | | | | | | | | | | | | | |
| G/G | 158 | 90 (63%) | 52 (37%) | | 10.8 (9.7, | 1 | | 1 | | 26.4 | 1 | | 1 | |
| | | | | | 12.3) | (Reference) | | (Reference) | | (24.2, | (Reference) | | (Reference) | |
| | | | | | | | | | | 29.1) | | | | |
| G/A^{a} | 33 | 22 (69%) | 10 (31%) | | 9.2 (6.3, | 1.50 (1.02, | | 1.58 (1.07, | | 21.2 | 1.33 (0.87, | | 1.51 (0.98, | |
| | | | | | 12.5) | 2.22) | | 2.35) | | (14.8, | 2.03) | | 2.32) | |
| | | | | | | | | | | 32.4) | | | | |
| A/A^{a} | 1 | 1 (100%) | 0 | | | | | | | | | | | |
| ACVR2B | | | | 0.63 | | | 0.58 | | 0.72 | | | 0.87 | | 0.78 |
| rs2268753 | | | | | | | | | | | | | | |
| T/T | 65 | 35 (58%) | 25 (42%) | | 10.7 (8.6, | 1 | | 1 | | 24.8 | 1 | | 1 | |
| | | | | | 12.5) | (Reference) | | (Reference) | | (21.0, | (Reference) | | (Reference) | |
| | | | | | | | | | | 28.6) | | | | |
| T/C | 78 | 48 (65%) | 26 (35%) | | 10.3 (9.0, | 0.94 (0.66, | | 1.03 (0.72, | | 25.4 | 0.91 (0.61, | | 1.02 (0.68, | |
| | | | | | 12.4) | 1.34) | | 1.48) | | (20.1, | 1.36) | | 1.53) | |
| | | | | | | | | | | 32.4) | | | | |
| C/C | 44 | 25 (68%) | 12 (32%) | | 9.7 (8.3, | 0.80 (0.52, | | 0.86 (0.55, | | 26.1 (17.4, | 0.91 | | 0.86 (0.53, | |
| | | | | | 13.5) | 1.23) | | 1.35) | | 33.2) | (0.56, 1.46) | | 1.41) | |
| MSTN | | | | 0.61 | | | 0.43 | | 0.67 | | | 0.29 | | 0.61 |
| rs7570532 | | | | | | | | | | | | | | |
| A/A | 103 | 58 (62%) | 36 (38%) | | 9.9 (8.7, | 1 | | 1 | | 24.8 | 1 | | 1 | |
| | | | | | 11.9) | (Reference) | | (Reference) | | (21.0, | (Reference) | | (Reference) | |
| | | | | | | | | | | 30.8) | | | | |
| A/G | 67 | 41 (68%) | 19 (32%) | | 10.3 | 0.81 | | 0.86 (0.61, | | 26.4 | 0.93 | | 0.98 (0.66, | |
| | | | | | (9.5, 13.1) | (0.58, 1.13) | | 1.21) | | (21.2, | (0.63, 1.36) | | 1.45) | |
| | | | | | | | | | | 28.8) | | | | |
| G/G | 10 | 5 (56%) | 4 (44%) | | 10.8 (3.1, | 1.05 (0.51, | | 0.85 (0.41, | | 18.4 (5.2, | 1.75 (0.80, | | 1.47 (0.67, | |
| | | | | | 14.9) | 2.18) | | 1.79) | | 28.0) | 3.84) | | 3.26) | |
| FOXO3 | | | | 0.43 | | | 0.35 | | 0.18 | | | 0.024 | | 0.038 |
| rs4946935 | | | | | | | | | | | | | | |
| G/G | 95 | 58 (66%) | 30 (34%) | | 10.0 (9.1, | 1 | | 1 | | 29.0 | 1 | | 1 | |
| | | | | | 11.7) | (Reference) | | (Reference) | | (23.2, | (Reference) | | (Reference) | |
| | | | | | | | | | | 33.3) | | | | |
| A/G | 72 | 44 (67%) | 22 (33%) | | 11.8 | 0.83 | | 0.76 (0.54, | | 23.8 | 1.29 | | 1.21 (0.81, | |
| | | | | | (9.1, 12.9) | (0.59, 1.16) | | 1.08) | | (21.2, | (0.88, 1.91) | | 1.80) | |
| | | | | | | | | | | 28.0) | | | | |
| A/A | 25 | 12 (52%) | 11 (48%) | | 9.2 (6.0, | 1.14 | | 1.13 (0.71, | | 15.1 (9.3, | 1.96 (1.18, | | 1.95 (1.17, | |
| | | | | | 13.0) | (0.72, 1.82) | | 1.82) | | 32.4) | 3.26) | | 3.27) | |

RAS mutant

| SMAD2 | | | | 0.55 | | | 0.53 | | 0.35 | | | 0.30 | | 0.13 |
|---|---|---|---|---|---|---|---|---|---|---|---|---|---|---|
| rs1792689 | | | | | | | | | | | | | | |
| G/G | 61 | 29 (49%) | 30 (51%) | | 10.1 (8.3, | 1 | | 1 (Reference) | | 17.5 | 1 (Reference) | | 1 (Reference) | |
| | | | | | 13.4) | (Reference) | | | | (15.9, | | | | |
| | | | | | | | | | | 22.1) | | | | |
| G/A^{b} | 15 | 8 (62%) | 5 (38%) | | 11.1 (8.3, | 0.83 (0.46, | | 0.74 (0.40, | | 26.5 | 0.71 | | 0.59 (0.30, | |
| | | | | | 17.1) | 1.50) | | 1.39) | | (18.5, | (0.37, 1.37) | | 1.17) | |
| | | | | | | | | | | 28.4) | | | | |
| A/A^{b} | 1 | 1 (100%) | 0 | | | | | | | | | | | |
| ACVR2B | | | | 0.33 | | | 0.83 | | 0.61 | | | 0.045 | | 0.036 |
| rs2268753 | | | | | | | | | | | | | | |
| T/T | 30 | 14 (48%) | 15 (52%) | | 8.8 (7.0, | 1 (Reference) | | 1 (Reference) | | 16.7 | 1 (Reference) | | 1 (Reference) | |
| | | | | | 12.2) | | | | | (13.7, | | | | |
| | | | | | | | | | | 23.1) | | | | |
| T/C | 36 | 18 (53%) | 16 (47%) | | 11.2 (8.5, | 0.88 (0.52, | | 0.75 (0.43, | | 26.3 | 0.64 (0.37, | | 0.50 (0.27, | |
| | | | | | 13.8) | 1.48) | | 1.33) | | (16.5, | 1.12) | | 0.91) | |
| | | | | | | | | | | 31.5) | | | | |
| C/C | 10 | 7 (78%) | 2 (22%) | | 12.0 | 0.82 (0.38, | | 0.92 (0.42, | | 41.9 (7.9, | 0.38 (0.15, | | 0.37 (0.14, | |
| | | | | | (1.3, 15.3) | 1.76) | | 2.02) | | 68.7) | 0.98) | | 1.02) | |
| | | | | 0.47 | | | 0.56 | | 0.38 | | | 0.029 | | 0.011 |
| T/T | 30 | 14 (48%) | 15 (52%) | | 8.8 (7.0, | 1 (Reference) | | 1 (Reference) | | 16.7 | 1 (Reference) | | 1 (Reference) | |
| | | | | | 12.2) | | | | | (13.7, | | | | |
| | | | | | | | | | | 23.1) | | | | |
| Any C | 46 | 25 (58%) | 18 (42%) | | 11.2 | 0.87 (0.53, | | 0.79 (0.46, | | 26.3 | 0.57 (0.34, | | 0.47 (0.26, | |
| | | | | | (8.9, 13.8) | 1.42) | | 1.34) | | (17.4, | 0.98) | | 0.84) | |
| | | | | | | | | | | 31.5) | | | | |
| MSTN | | | | 0.74 | | | 0.071 | | 0.089 | | | 0.020 | | 0.047 |
| rs7570532 | | | | | | | | | | | | | | |
| A/A | 48 | 23 (51%) | 22 (49%) | | 10.1 (8.2, | 1 | | 1 (Reference) | | 26.7 | 1 (Reference) | | 1 (Reference) | |
| | | | | | 13.8) | (Reference) | | | | (16.7, | | | | |
| | | | | | | | | | | 32.8) | | | | |
| A/G | 19 | 11 (61%) | 7 (39%) | | 8.7 (6.1, | 1.70 (0.96, | | 1.75 (0.98, | | 16.5 | 1.85 (1.02, | | 1.91 (1.04, | |
| | | | | | 12.3) | 3.01) | | 3.11) | | (11.2, | 3.33) | | 3.51) | |
| | | | | | | | | | | 20.6) | | | | |
| G/G | 10 | 6 (60%) | 4 (40%) | | 13.9 (8.0, | 0.79 (0.38, | | 0.79 (0.37, | | 22.1 (13.3, | 0.66 | | 0.73 (0.29, | |
| | | | | | 17.2) | 1.63) | | 1.69) | | 68.7) | (0.28, 1.56) | | 1.83) | |
| FOXO3 | | | | 0.12 | | | 0.58 | | 0.32 | | | 0.60 | | 0.30 |
| rs4946935 | | | | | | | | | | | | | | |
| G/G | 38 | 19 (54%) | 16 (46%) | | 10.3 (8.5, | 1 (Reference) | | 1 (Reference) | | 20.6 | 1 (Reference) | | 1 (Reference) | |
| | | | | | 13.8) | | | | | (16.5, | | | | |
| | | | | | | | | | | 29.0) | | | | |
| A/G c | 34 | 19 (58%) | 14 (42%) | | 9.7 (7.8, | 1.14 (0.71, | | 1.30 (0.77, | | 20.6 | 1.14 | | 1.34 (0.77, | |
| | | | | | 12.7) | 1.84) | | 2.19) | | (16.4, | (0.68, 1.93) | | 2.33) | |
| | | | | | | | | | | 26.5) | | | | |
| A/A c | 4 | 0 | 4 (100%) | | | | | | | | | | | |

Abbreviations:
CI, confidence interval;
HR, hazard ratio.
Significant P values are indicated in bold characters.
^{a,b,c}Grouped together for the estimates of HR.
*P value was based on Fisher's exact test for tumor response, log-rank test for progression free survival and overall survival in the univariate analysis (†), and Wald test in the multivariable Cox proportional hazards regression model (‡) adjusted for sex, ECOG performance status, liver metastasis, resection of the primary tumors.

SUPPLEMENTAL TABLE 2

| | | | | | Median, | | | | | Median, | | | | |
|---|---|---|---|---|---|---|---|---|---|---|---|---|---|---|
| Gene rs number | | Tumor response | | | months | Progression-free Survival | | | | months | Overall Survival | | | P |
| Genotype | N | PR + CR | SD + PD | P value* | (95% CI) | HR (95% CI) † | P value* | HR (95% CI) ‡ | P value* | (95% CI) | HR (95% CI) † | P value* | HR (95% CI) ‡ | Value* |
| INHBA | | | | 0.26 | | | 0.25 | | 0.31 | | | 0.25 | | 0.84 |
| rs17776182 | | | | | | | | | | | | | | |
| G/G | 187 | 104 (62%) | 64 (38%) | | 10.3 (9.3, 11.8) | 1 | | 1 (Reference) | | 24.2 (21.2, | 1 (Reference) | | 1 (Reference) | |
| | | | | | | (Reference) | | | | 27.6) | | | | |
| A/G | 71 | 43 (62%) | 26 (38%) | | 10.1 (8.3, 12.0) | 1.20 (0.90, | | 1.22 (0.91, 1.64) | | 24.7 (19.5, | 0.95 (0.69, | | 1.00 (0.72, | |
| | | | | | | 1.61) | | | | 28.4) | 1.32) | | 1.38) | |
| A/A | 12 | 4 (36%) | 7 (64%) | | 8.8 (2.9, 13.1) | 1.50 (0.79, | | 1.38 (0.70, 2.72) | | 23.1 (6.3, | 1.71 (0.86, | | 1.24 (0.60, | |
| | | | | | | 2.86) | | | | 29.0) | 3.38) | | 2.56) | |
| INHBA | | | | 0.46 | | | 0.68 | | 0.88 | | | 0.58 | | 0.67 |
| rs2237432 | | | | | | | | | | | | | | |
| T/T | 168 | 96 (62%) | 58 (38%) | | 10.2 (9.2, 12.3) | 1 | | 1 (Reference) | | 23.7 (21.0, | 1 (Reference) | | 1 (Reference) | |
| | | | | | | (Reference) | | | | 28.0) | | | | |
| T/C | 92 | 51 (59%) | 35 (41%) | | 9.9 (8.8, 11.3) | 1.08 (0.82, | | 1.07 (0.81, 1.42) | | 23.7 (18.9, | 1.08 (0.79, | | 0.97 (0.71, | |
| | | | | | | 1.43) | | | | 26.4) | 1.46) | | 1.32) | |
| C/C | 16 | 7 (47%) | 8 (53%) | | 12.9 (4.4, 16.6) | 0.85 (0.49, | | 0.98 (0.56, 1.71) | | 28.6 (13.9, | 0.77 (0.41, | | 0.75 (0.40, | |
| | | | | | | 1.47) | | | | 43.7) | 1.43) | | 1.41) | |
| MSTN | | | | 0.57 | | | 0.72 | | 0.64 | | | 0.81 | | 0.80 |
| rs7570532 | | | | | | | | | | | | | | |
| A/A | 157 | 85 (59%) | 59 (41%) | | 9.9 (8.9, 11.5) | 1 | | 1 (Reference) | | 24.7 (20.6, | 1 (Reference) | | 1 (Reference) | |
| | | | | | | (Reference) | | | | 28.6) | | | | |
| A/G | 88 | 52 (65%) | 28 (35%) | | 10.3 (9.0, 11.7) | 0.92 (0.69, | | 0.97 (0.73, | | 23.1 (18.4, | 1.03 (0.75, | | 1.09 (0.79, | |
| | | | | | | 1.22) | | 1.29) | | 26.4) | 1.40) | | 1.49) | |
| G/G | 21 | 11 (55%) | 9 (45%) | | 13.5 (8.0, 14.9) | 0.84 (0.51, | | 0.78 (0.47, | | 22.1 (15.9, | 0.86 (0.48, | | 0.91 (0.50, | |
| | | | | | | 1.40) | | 1.30) | | 30.8) | 1.53) | | 1.63) | |
| ALK4rs2854464 | | | | 0.57 | | | 0.58 | | 0.42 | | | 0.26 | | 0.13 |
| T/T | 158 | 84 (59%) | 59 (41%) | | 9.9 (8.8, 10.5) | 1 | | 1 (Reference) | | 22.7 (18.7, | 1 (Reference) | | 1 (Reference) | |
| | | | | | | (Reference) | | | | 25.9) | | | | |
| T/C | 107 | 65 (64%) | 37 (36%) | | 11.3 (9.6, 12.8) | 0.87 (0.67, | | 0.83 (0.64, | | 25.0 (21.2, | 0.86 (0.64, | | 0.83 (0.62, | |
| | | | | | | 1.14) | | 1.09) | | 28.6) | 1.15) | | 1.12) | |
| C/C | 22 | 14 (70%) | 6 (30%) | | 9.5 (7.8, 12.7) | 0.95 (0.59, | | 0.89 (0.56, | | 29.1 (16.5, | 0.67 (0.38, | | 0.58 (0.33, | |
| | | | | | | 1.52) | | 1.44) | | 58.7) | 1.17) | | 1.04) | |
| TGFBR1rs10760673 | | | | 0.51 | | | 0.82 | | 0.95 | | | 0.60 | | 0.72 |
| G/G | 181 | 107 (64%) | 60 (36%) | | 10.2 (9.1, 12.2) | 1 | | 1 (Reference) | | 25.4 (21.0, | 1 (Reference) | | 1 (Reference) | |
| | | | | | | (Reference) | | | | 27.5) | | | | |
| A/G^{a} | 84 | 45 (57%) | 34 (43%) | | 10.3 (9.7, 11.8) | 1.03 (0.78, | | 0.99 (0.75, | | 23.8 (21.2, | 1.09 (0.80, | | 1.06 (0.78, | |
| | | | | | | 1.36) | | 1.31) | | 28.1) | 1.48) | | 1.44) | |
| A/A^{a} | 4 | 2 (50%) | 2 (50%) | | | | | | | | | | | |
| ALK7 | | | | 0.38 | | | 0.24 | | 0.40 | | | 0.15 | | 0.20 |
| rs13010956 | | | | | | | | | | | | | | |
| T/T | 95 | 47 (55%) | 39 (45%) | | 11.1 (10.1, 12.7) | 1 | | 1 (Reference) | | 26.1 (21.3, | 1 (Reference) | | 1 (Reference) | |
| | | | | | | (Reference) | | | | 30.8) | | | | |
| T/C | 127 | 72 (62%) | 44 (38%) | | 9.7 (8.9, 12.2) | 0.99 (0.74, | | 0.97 (0.73, | | 21.2 (18.4, | 1.32 (0.95, | | 1.26 (0.90, | |
| | | | | | | 1.32) | | 1.31) | | 24.7) | 1.83) | | 1.76) | |
| C/C | 52 | 33 (66%) | 33 (66%) | | 9.7 * 8.3, 11.3) | 1361 (0.91, | | 1.24 (0.85, | | 26.7 (23.0, | 0.99 (0.66, | | 0.93 (0.61, | |
| | | | | | | 1.88) | | 1.79) | | 34.6) | 1.50) | | 1.41) | |
| ACVR2Brs13072731 | | | | 0.46 | | | 0.57 | | 0.57 | | | 0.27 | | 0.41 |
| C/C | 107 | 57 (57%) | 43 (43%) | | 10.2 (8.8, 11.7) | 1 | | 1 (Reference) | | 24.7 (19.0, | 1 (Reference) | | 1 (Reference) | |
| | | | | | | (Reference) | | | | 27.4) | | | | |
| C/A | 124 | 71 (61%) | 45 (39%) | | 10.3 (9.0, 12.3) | 0.95 (0.72, | | 0.89 (0.67, | | 23.8 (20.6, | 1.24 (0.90, | | 1.19 (0.86, | |
| | | | | | | 1.25) | | 1.18) | | 28.0) | 1.70) | | 1.63) | |
| A/A | 50 | 30 (68%) | 14 (32%) | | 9.8 (8.9, 14.3) | 0.82 (0.56, | | 0.83 (0.56, | | 23.1 (17.6, | 0.97 (0.63, | | 0.94 (0.61, | |
| | | | | | | 1.20) | | 1.22) | | 41.9) | 1.49) | | 1.44) | |
| ACVR2Brs2268753 | | | | 0.24 | | | 0.52 | | 0.75 | | | 0.39 | | 0.35 |
| T/T | 101 | 52 (55%) | 42 (45%) | | 9.9 (8.6, 11.7) | 1 | | 1 (Reference) | | 23.1 (18.5, | 1 (Reference) | | 1 (Reference) | |
| | | | | | | (Reference) | | | | 25.9) | | | | |
| T/C | 118 | 68 (61%) | 44 (39%) | | 10.3 (9.2, 12.3) | 0.95 (0.72, | | 0.97 (0.73, | | 24.8 (21.5, | 0.90 (0.66, | | 0.96 (0.70, | |
| | | | | | | 1.27) | | 1.30) | | 28.4) | 1.23) | | 1.32) | |
| C/C | 55 | 33 (70%) | 14 (30%) | | 10.1 (8.9, 13.5) | 0.81 (0.56, | | 0.87 (0.60, | | 26.1 (17.6, | 0.75 (0.50, | | 0.74 (0.48, | |
| | | | | | | 1.17) | | 1.26) | | 41.9) | 1.13) | | 1.12) | |
| SMAD2 | | | | 0.97 | | | 0.66 | | 0.54 | | | 0.79 | | 0.61 |
| rs1792671 | | | | | | | | | | | | | | |
| C/C | 83 | 45 (62%) | 28 (38%) | | 9.7 (8.1, 11.3) | 1 | | 1 (Reference) | | 22.3 (17.5, | 1 (Reference) | | 1 (Reference) | |
| | | | | | | (Reference) | | | | 26.1) | | | | |
| C/T | 118 | 69 (61%) | 44 (39%) | | 10.3 (9.2, 12.0) | 0.94 (0.70, | | 0.98 (0.72, | | 24.8 (21.2, | 0.94 (0.67, | | 1.08 (0.76, | |
| | | | | | | 1.28) | | 1.34) | | 28.8) | 1.32) | | 1.53) | |
| T/T | 66 | 38 (63%) | 22 (37%) | | 10.8 (8.5, 13.4) | 0.85 (0.60, | | 0.83 (0.59, | | 23.1 (19.5, | 0.87 (0.59, | | 0.89 (0.60, | |
| | | | | | | 1.21) | | 1.18) | | 30.3) | 1.29) | | 1.32) | |
| SMAD2 | | | | 0.31 | | | 0.27 | | 0.20 | | | 0.38 | | 0.34 |
| rs1792689 | | | | | | | | | | | | | | |
| G/G | 230 | 125 (59%) | 86 (41%) | | 10.5 (9.7, 11.8) | 1 | | 1 (Reference) | | 24.8 (21.9, | 1 (Reference) | | 1 (Reference) | |
| | | | | | | (Reference) | | | | 27.6) | | | | |
| G/A^{b} | 48 | 30 (67%) | 15 (33%) | | 9.7 (8.1, 12.5) | 1.20 (0.86, | | 1.24 (0.89, | | 23.6 (18.4, | 1.17 (0.82, | | 1.19 (0.83, | |
| | | | | | | 1.65) | | 1.72) | | 28.4) | 1.65) | | 1.69) | |
| A/A^{b} | 3 | 3 (100%) | 0 | | | | | | | | | | | |
| FOXO3 | | | | 0.80 | | | 0.27 | | 0.14 | | | 0.80 | | 0.68 |
| rs12212067 | | | | | | | | | | | | | | |
| T/T | 209 | 111 (59%) | 78 (41%) | | 9.8 (8.9, 10.5) | 1 | | 1 (Reference) | | 23.0 (19.4, | 1 (Reference) | | 1 (Reference) | |
| | | | | | | (Reference) | | | | 26.4) | | | | |
| T/G c | 54 | 32 (62%) | 20 (38%) | | 11.2 (9.0, 13.5) | 0.84 (0.62, | | 0.79 (0.58, | | 25.0 (21.2, | 0.96 (0.68, | | 0.93 (0.66, | |
| | | | | | | 1.15) | | 1.08) | | 28.0) | 1.35) | | 1.31) | |
| G/G c | 7 | 5 (71%) | 2 (29%) | | | | | | | | | | | |
| FOXO3 | | | | 0.26 | | | 0.58 | | 0.45 | | | 0.085 | | 0.12 |
| rs4946935 | | | | | | | | | | | | | | |
| G/G | 138 | 80 (63%) | 47 (37%) | | 10.1 (9.2, 11.5) | 1 | | 1 (Reference) | | 26.7 (21.3, | 1 (Reference) | | 1 (Reference) | |
| | | | | | | (Reference) | | | | 30.3) | | | | |
| A/G | 111 | 65 (63%) | 39 (38%) | | 11.2 (8.9, 12.3) | 0.88 (0.67, | | 0.84 (0.64, | | 23.7 (21.2, | 1.11 (0.81, | | 1.08 (0.79, | |
| | | | | | | 1.16) | | 1.12) | | 26.4) | 1.51) | | 1.47) | |
| A/A | 30 | 13 (46%) | 15 (54%) | | 9.5 (6.5, 13.0) | 1.05 (0.69, | | 1.01 (0.66, 1.55) | | 20.1 (11.3, | 1.65 (1.05, | | 1.60 (1.02, | |
| | | | | | | 1.59) | | | | 28.6) | 2.59) | | 2.53) | |
| | | | | 0.10 | | | 0.61 | | 0.66 | | | 0.034 | | 0.046 |
| Any G | 249 | 145 (63%) | 86 (37%) | | 10.3 (9.6, 11.7) | 1 | | 1 (Reference) | | 24.8 (21.9, | 1 (Reference) | | 1 (Reference) | |
| | | | | | | (Reference) | | | | 27.5) | | | | |
| A/A | 30 | 13 (46%) | 15 (54%) | | 9.5 (6.5, 13.0) | 1.11 (0.74, | | 1.10 (0.73, | | 20.1 (11.3, | 1.58 (1.03, | | 1.55 (1.01, | |
| | | | | | | 1.66) | | 1.64) | | 28.6) | 2.43) | | 2.39) | |

### Associations Between Cachexia SNPs and Outcomes Stratified by Tumor RAS Mutation Status in the Training Cohort.

Activation of the RAS pathway has been linked with the development of cachexia in cancer patients [25]. Associations between cachexia related SNPs and outcomes was influenced by tumor RAS mutation status (Table 3).

TABLE 3
Association between cachexia-related gene polymorphisms and clinical outcomes according to RAS mutant status
in the validation cohort (TRIBE cohort).

| Gene rs | | | | | Median, | | | | | Median, | Overall Survival | | | |
|---|---|---|---|---|---|---|---|---|---|---|---|---|---|---|
| number | | Tumor response | | | months | Progression-free Survival | | | | months | HR | | | |
| Genotype | N | PR + CR | SD + PD | P value* | (95% CI) | HR (95% CI)† | P value* | HR (95% CI)‡ | P value* | (95% CI) | (95% CI)† | P value* | HR (95% CI)‡ | P value* |

RAS wild-type

| SMAD2 | | | | 0.44 | | | 0.64 | | 0.55 | | | 0.97 | | 0.59 |
|---|---|---|---|---|---|---|---|---|---|---|---|---|---|---|
| rs1792689 | | | | | | | | | | | | | | |
| G/G | 46 | 25 (57%) | 19 (43%) | | 10.8 | 1 | | 1 | | 25.7 | 1 | | 1 | |
| | | | | | (8.1, 12.2) | (Reference) | | (Reference) | | (18.4, | (Reference) | | (Reference) | |
| | | | | | | | | | | 35.8) | | | | |
| G/A | 8 | 3 (38%) | 5 (63%) | | 12.3 (2.0, | 0.82 (0.36, | | 0.76 (0.30, | | 26.8 (3.5, | 1.01 (0.45, | | 1.28 (0.52, | |
| | | | | | 17.8) | 1.88) | | 1.91) | | 57.7) | 2.28) | | 3.14) | |
| A/A | 1 | 1 (100%) | 0 | | | | | | | | | | | |
| ACVR2B | | | | 0.71 | | | 0.15 | | 0.15 | | | 0.77 | | 0.82 |
| rs2268753 | | | | | | | | | | | | | | |
| T/T | 16 | 10 (63%) | 6 (38%) | | 11.2 (7.9, | 1 | | 1 | | 33.7 | 1 | | 1 | |
| | | | | | 14.3) | (Reference) | | (Reference) | | (18.4, | (Reference) | | (Reference) | |
| | | | | | | | | | | 52.5) | | | | |
| T/C | 27 | 13 (48%) | 14 (52%) | | 10.8 (3.0, | 1.44 (0.68, | | 1.72 (0.67, | | 24.8 | 1.29 (0.63, | | 1.21 (0.51, | |
| | | | | | 12.4) | 3.03) | | 4.45) | | (11.0, | 2.64) | | 2.84) | |
| | | | | | | | | | | 35.8) | | | | |
| C/C | 12 | 6 (60%) | 4 (40%) | | 11.3 (2.8, | 0.71 (0.28, | | 0.64 (0.19, | | 32.7 (2.8, | 1.24 (0.53, | | 0.94 (0.34, | |
| | | | | | 30.8) | 1.83) | | 2.21) | | 48.6) | 2.87) | | 2.62) | |
| MSTN | | | | 0.39 | | | 0.73 | | 0.67 | | | 0.92 | | 0.95 |
| rs7570532 | | | | | | | | | | | | | | |
| A/A | 28 | 14 (54%) | 12 (46%) | | 11.0 | 1 | | 1 | | 31.1 (15.1, | 1 | | 1 | |
| | | | | | (7.9, 12.7) | (Reference) | | (Reference) | | 37.8) | (Reference) | | (Reference) | |
| A/G | 24 | 12 (50%) | 12 (50%) | | 10.8 (7.7, | 1.11 (0.59, | | 1.17 (0.57, | | 24.8 | 1.03 (0.56, | | 0.98 (0.50, | |
| | | | | | 12.6) | 2.09) | | 2.39) | | (11.2, | 1.89) | | 1.92) | |
| | | | | | | | | | | 49.1) | | | | |
| G/G | 3 | 3 (100%) | 0 | | | | | | | | | | | |
| FOXO3 | | | | 0.16 | | | 0.60 | | 0.34 | | | 0.79 | | 0.70 |
| rs4946935 | | | | | | | | | | | | | | |
| G/G | 31 | 18 (60%) | 12 (40%) | | 10.8 (7.9, | 1 | | 1 | | 22.3 | 1 | | 1 | |
| | | | | | 12.4) | (Reference) | | (Reference) | | (11.2, | (Reference) | | (Reference) | |
| | | | | | | | | | | 35.8) | | | | |
| A/G | 18 | 10 (59%) | 7 (41%) | | 11.3 (7.7, | 0.75 (0.37, | | 0.88 (0.40, | | 35.0 | 0.82 (0.42, | | 1.11 (0.53, | |
| | | | | | 14.3) | 1.55) | | 1.94) | | (18.4, | 1.60) | | 2.35) | |
| | | | | | | | | | | 50.2) | | | | |
| A/A | 6 | 1 (17%) | 5 (83%) | | 12.3 (8.1, | 0.68 (0.23, | | 0.38 | | 25.8 | 1.11 (0.42, | | 1.63 (0.52, | |
| | | | | | 13.7) | 2.00) | | (0.10, 1.38) | | (14.6, | 2.95) | | 5.08) | |
| | | | | | | | | | | 40.7) | | | | |

RAS mutant

| SMAD2 | | | | 0.13 | | | 0.80 | | 0.35 | | | 0.82 | | 0.36 |
|---|---|---|---|---|---|---|---|---|---|---|---|---|---|---|
| rs1792689 | | | | | | | | | | | | | | |
| G/G | 91 | 48 (55%) | 40 (45%) | | 9.6 (9.0, | 1 (Reference) | | 1 (Reference) | | 24.0 | 1 | | 1 (Reference) | |
| | | | | | 11.1) | | | | | (20.5, | (Reference) | | | |
| | | | | | | | | | | 31.6) | | | | |
| G/A | 24 | 17 (71%) | 7 (29%) | | 8.7 (7.5, | 1.06 (0.66, | | 1.28 (0.76, | | 25.2 | 1.06 (0.64, | | 1.28 (0.75, | |
| | | | | | 11.5) | 1.72) | | 2.14) | | (16.3, | 1.74) | | 2.17) | |
| | | | | | | | | | | 44.1) | | | | |
| A/A | 1 | 0 | 1 (100%) | | | | | | | | | | | |
| ACVR2B | | | | 0.034 | | | 0.15 | | 0.019 | | | 0.96 | | 0.65 |
| rs2268753 | | | | | | | | | | | | | | |
| T/T | 30 | 14 (48%) | 15 (52%) | | 9.2 (7.6, | 1 (Reference) | | 1 (Reference) | | 26.2 | 1 | | 1 (Reference) | |
| | | | | | 11.1) | | | | | (18.1, | (Reference) | | | |
| | | | | | | | | | | 37.1) | | | | |
| T/C | 55 | 37 (70%) | 16 (30%) | | 9.7 | 0.68 (0.41, | | 0.51 (0.30, | | 24.8 | 0.96 (0.58, | | 0.77 (0.45, | |
| | | | | | (8.8, 12.7) | 1.12) | | 0.87) | | (18.3, | 1.59) | | 1.32) | |
| | | | | | | | | | | 32.5) | | | | |
| C/C | 30 | 13 (43%) | 17 (57%) | | 9.2 (7.5, | 1.05 (0.60, | | 0.97 (0.54, | | 23.4 | 0.92 (0.52, | | 0.85 (0.47, | |
| | | | | | 10.8) | 1.84) | | 1.74) | | (20.0, | 1.62) | | 1.54) | |
| | | | | | | | | | | 38.3) | | | | |
| | | | | 0.28 | | | 0.30 | | 0.068 | | | 0.81 | | 0.38 |
| T/T | 30 | 14 (48%) | 15 (52%) | | 9.2 (7.6, | 1 (Reference) | | 1 (Reference) | | 26.2 | 1 | | 1 (Reference) | |
| | | | | | 11.1) | | | | | (18.1, | (Reference) | | | |
| | | | | | | | | | | 37.1) | | | | |
| Any C | 85 | 50 (60%) | 33 (40%) | | 9.5 (8.8, | 0.78 (0.49, | | 0.64 | | 23.9 | 0.94 (0.59, | | 0.80 (0.49, | |
| | | | | | 11.1) | 1.24) | | (0.39, 1.03) | | (19.8, | 1.51) | | 1.31) | |
| | | | | | | | | | | 28.6) | | | | |
| MSTN | | | | 0.39 | | | 0.43 | | 0.35 | | | 0.21 | | 0.14 |
| rs7570532 | | | | | | | | | | | | | | |
| A/A | 71 | 43 (61%) | 27 (39%) | | 9.7 (8.8, | 1 (Reference) | | 1 (Reference) | | 24.8 | 1 | | 1 (Reference) | |
| | | | | | 11.1) | | | | | (19.8, | (Reference) | | | |
| | | | | | | | | | | 37.1) | | | | |
| A/G | 41 | 19 (49%) | 20 (51%) | | 9.2 | 1.15 (0.75, | | 1.33 (0.84, | | 22.4 | 1.33 (0.87, | | 1.49 (0.94, | |
| | | | | | (7.6, 11.1) | 1.77) | | 2.10) | | (17.8, | 2.05) | | 2.35) | |
| | | | | | | | | | | 28.6) | | | | |
| G/G | 4 | 3 (75%) | 1 (25%) | | 12.7 (6.7, | 0.56 (0.18, | | 0.69 (0.21, | | (12.5, | 0.50 (0.12, | | 0.54 (0.13, | |
| | | | | | 55.0) | 1.80) | | 2.25) | | 55.0) | 2.06) | | 2.24) | |
| FOXO3 | | | | 0.24 | | | 0.77 | | 0.56 | | | 0.35 | | 0.68 |
| rs4946935 | | | | | | | | | | | | | | |
| G/G | 46 | 22 (48%) | 24 (52%) | | 9.0 (7.6, | 1 (Reference) | | 1 (Reference) | | 26.1 (19.0, | 1 | | 1 (Reference) | |
| | | | | | 11.5) | | | | | 42.7) | (Reference) | | | |
| A/G | 59 | 37 (65%) | 20 (35%) | | 9.6 | 1.06 (0.69, | | 0.83 (0.52, | | 23.0 | 1.35 (0.86, | | 0.93 (0.57, | |
| | | | | | (8.6, 11.6) | 1.65) | | 1.33) | | (19.7, | 2.10) | | 1.51) | |
| | | | | | | | | | | 31.6) | | | | |
| A/A | 10 | 6 (60%) | 4 (40%) | | 9.2 (2.8, | 0.80 (0.35, | | 0.64 (0.25, | | 23.2 | 0.96 (0.42, | | 0.65 (0.25, | |
| | | | | | 28.8) | 1.81) | | 1.66) | | (12.6, | 2.17) | | 1.70) | |
| | | | | | | | | | | 65.3) | | | | |

Abbreviations:
CI, confidence interva;
HR, hazard ratio.
Significant P values are indicated in bold characters.
*P value was based on Fisher's exact test for tumor response, log-rank test for progression free survival and overall survival in the univariate analysis (†), and Wald test in the multivariable Cox proportional hazards regression model (‡) adjusted for age, ECOG performance status, primary tumor site, the number of metastatic sites, BRAF mutation status, resection of the primary tumors, and adjuvant therapy

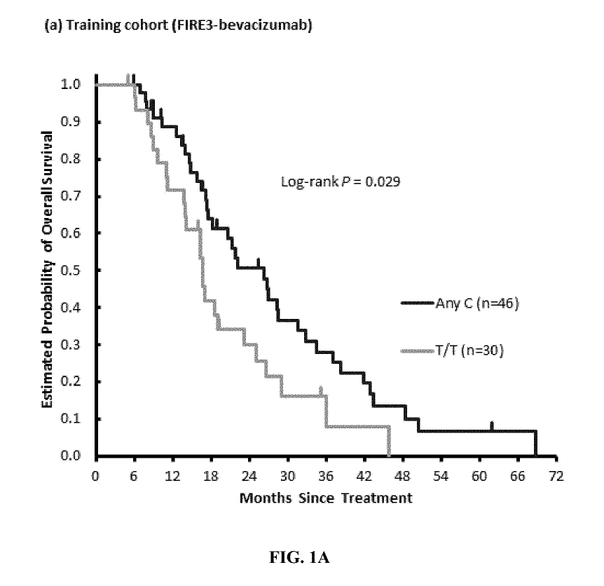

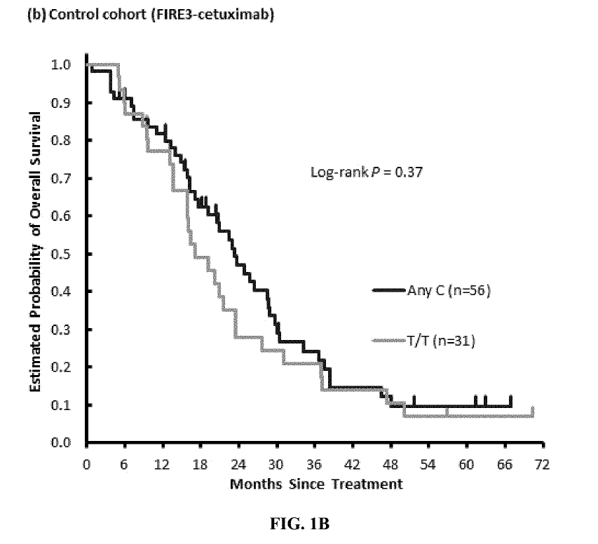

Among patients with RAS mutant tumors in the FIRE-3 training cohort, those with the ACVR2B rs2268753 C/C variant had the longest OS (41.9 months) compared to those with the T/C (26.3 months) or T/T genotypes (16.7 months), both in univariable (P=0.045) and multivariable (P=0.036) analyses. Patients with the ACVR2B rs2268753 any C variant had a significantly longer OS than those with T/T allele (26.3 vs. 16.7 months) in both univariable (HR 0.57, 95% CI 0.34-0.98, P=0.029) and multivariable (HR 0.47, 95% CI 0.26-0.84, P=0.011) analyses (Table 3, FIG. 1A). In addition, patients with RAS mutant tumors of the FIRE-3 training cohort who carried the MSTN rs7570532 A/G variant had a significantly shorter OS (16.5 months) compared to those with the A/A variant (26.7 months), both in univariable (HR 1.85, 95% CI 1.02-3.33, P=0.02) and multivariable (HR 1.91, 95% CI 1.04-3.51, P=0.047) analyses (Table 3). There was no association between ACVR2B rs2268753 or MSTN rs7570532 genotype and outcomes in patients with RAS wildtype cancers of the training cohort.

Among patients with RAS wildtype cancers, those with the SMAD2 rs1792689 G/A variant had an inferior PFS (9.2 months) compared to those with the G/G variant (10.8 months), in univariable (HR 1.50, 95% CI 1.02-2.22, P=0.036) and multivariable (HR 1.58, 95% CI 1.07-2.35, P=0.022) analyses (Table 3). In addition, those carrying the A/A variant of FOXO3 rs4946935 had the shortest OS (15.1 months) compared to those with the A/G (23.8 months) or G/G polymorphisms (29.0 months), both in univariable (P=0.024) and multivariable analysis (P=0.038). These associations were not observed in the RAS mutant subgroup of the training cohort (Table 3).

### Associations Between Cachexia SNPs and Outcomes Stratified by Tumor RAS Mutation Status in the Validation Cohort

Similar to the training cohort, ACVR2B rs2268753 genotype was significantly associated with outcomes in RAS mutant but not wildtype patients within the TRIBE validation cohort (Table 4). Specifically, patients with RAS mutant tumors and the ACVR2B rs2268753 T/C genotype had a superior RR (70%) and PFS (9.7 months) compared to those with the T/T variant (RR 48%, PFS 9.2 months; multivariable HR for PFS 0.51, P=0.019). There was no significant relationship between ACVR2B rs2268753 variants and OS in RAS mutant patients of the TRIBE validation cohort.

TABLE 4
Association between ACVR2B rs2268753 and clinical outcomes in RAS
mutant patients of control cohort (FIRE3-cetuximab cohort).

| Gene rs | | Tumor response | | | Median, | Progression-free Survival | | | |
|---|---|---|---|---|---|---|---|---|---|
| number | | | | P | months | HR | P | | P |
| Genotype | N | PR + CR | SD + PD | value* | (95% CI) | (95% CI)† | value* | HR (95% CI)‡ | value* |
| ACVR2B | | | | 0.76 | | | 0.21 | | 0.29 |
| rs2268753 | | | | | | | | | |
| T/T | 31 | 13 | 15 | | 7.9 (5.7, | 1 | | 1 (Reference) | |
| | | (46%) | (54%) | | 10.0) | (Reference) | | | |
| T/C | 35 | 14 | 17 | | 8.5 (6.0, | 0.81 (0.49, | | 0.80 (0.47, | |
| | | (45%) | (55%) | | 10.9) | 1.34) | | 1.34) | |
| C/C | 21 | 6 | 11 | | 5.3 (3.0, 9.0) | 1.32 (0.74, | | 1.26 (0.69, | |
| | | (35%) | (65%) | | | 2.36) | | 2.28) | |
| | | | | 0.81 | | | 0.82 | | 0.75 |
| T/T | 31 | 13 | 15 | | 7.9 (5.7, | 1 | | 1 (Reference) | |
| | | (46%) | (54%) | | 10.0) | (Reference) | | | |
| Any C | 56 | 20 | 28 | | 6.9 (5.3, 9.6) | 0.95 (0.60, | | 0.93 (0.57, | |
| | | (42%) | (58%) | | | 1.50) | | 1.49) | |

| Gene rs | | Median, | Overall Survival | | | |
|---|---|---|---|---|---|---|
| number | | months | HR | P | HR | P |
| Genotype | N | (95% CI) | (95% CI)† | value* | (95% CI)‡ | value* |
| ACVR2B | | | | 0.33 | | 0.31 |
| rs2268753 | | | | | | |
| T/T | 31 | 17.1 | 1 | | 1 | |
| | | (13.6, | (Reference) | | (Reference) | |
| | | 23.5) | | | | |
| T/C | 35 | 25.7 | 0.71 (0.41, | | 0.66 (0.38, | |
| | | (20.6, | 1.21) | | 1.16) | |
| | | 28.8) | | | | |
| C/C | 21 | 17.7 | 1.04 (0.56, | | 0.94 (0.50, | |
| | | (14.0, | 1.94) | | 1.76) | |
| | | 30.4) | | | | |
| | | | | 0.37 | | 0.26 |
| T/T | 31 | 17.1 | 1 | | 1 | |
| | | (13.6, | (Reference) | | (Reference) | |
| | | 23.5) | | | | |
| Any C | 56 | 23.4 | 0.80 (0.50, | | 0.75 (0.45, | |
| | | (17.7, | 1.30) | | 1.24) | |
| | | 28.7) | | | | |

Abbreviations:
CI, confidence interval;
HR, hazard ratio.
Significant P values are indicated in bold characters.
*P value was based on Fisher's exact test for tumor response, log-rank test for progression free survival and overall survival in the univariate analysis (†), and Wald test in the multivariable Cox proportional hazards regression model (‡) adjusted for sex, ECOG performance status, liver metastasis, resection of the primary tumors.

No significant associations were observed between any of the tested SNPs and outcomes in RAS wildtype patients of the TRIBE validation cohort.

Associations Between Cachexia SNPs and Outcomes in the Control Cohort.

Within the FIRE3-cetuximab control cohort, there were no significant associations between the examined cachexia SNPs and outcomes (Table 3, FIG. 1B).

Association Between ACVR2B rs2268753 Genotype and Gene Expression.

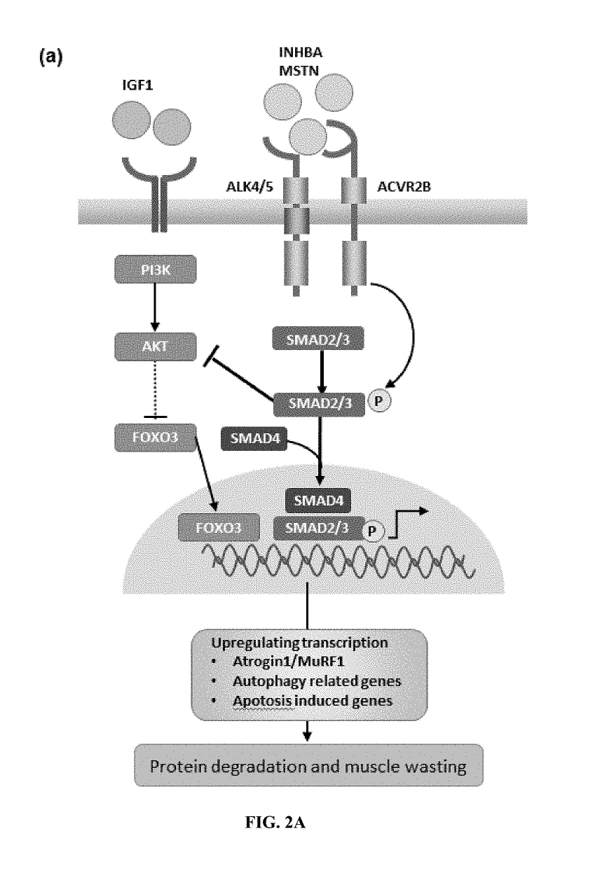

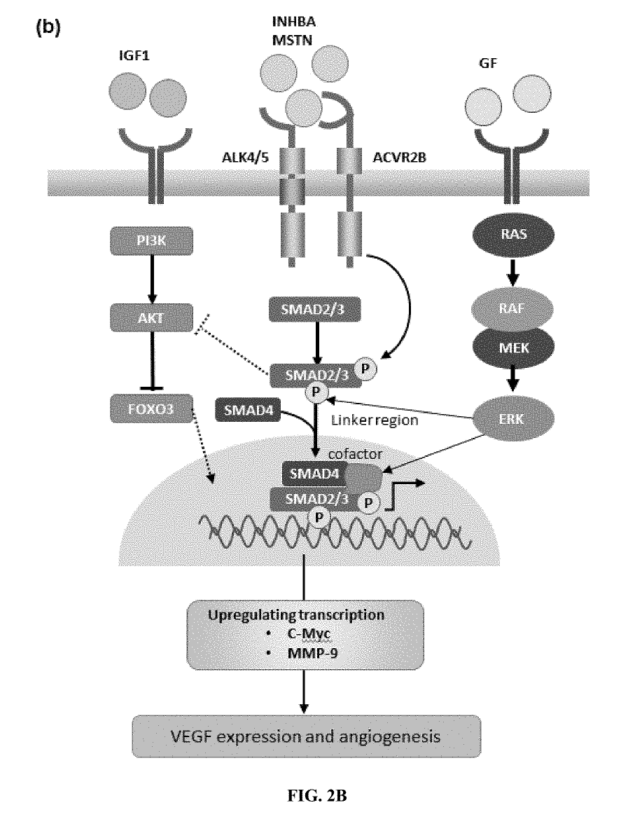

ACVR2B and VEGF gene expressions were evaluated in relation to ACVR2B rs2268753 genotype in tumors from 43 patients. Tumor ACVR2B gene expression was significantly higher in ACVR2B rs2268753 C/C patients than in those with C/T or T/T genotypes (P=0.033) (FIG. 2A). ACVR2B rs2268753 genotype didn't influence tumor VEGF expression (FIG. 2B).

### DISCUSSION

Activin/myostatin signaling serves as a critical pathway linking cancer associated cachexia with angiogenesis with potential impact of bevacizumab efficacy. The clinical relevance of genetic polymorphisms within these pathways and benefit from anti-angiogenic cancer therapy is unknown. This example demonstrated that germline variants within the cancer cachexia pathway are associated with outcomes in mCRC patients treated with bevacizumab-based chemotherapy which is dependend from tumor RAS mutation status and not seen in patients treated with Cetuximab based chemotherapy with wt ras tumors.

Activin/myostatin signaling is directly involved in skeletal muscle degradation [7] by binding to type II receptor (ACVR2B) and inducing its dimerization with the activin type I receptor. Subsequent phosphorylation of Smad2/3 recruits Smad4 [26]. This Smad complex is then translocated into the nucleus to induce transcriptional changes, which result in muscle degradation. In addition, activin/myostatin suppresses Akt activity and as a result, FOXO3 phosphorylation [27]. Dephosphorylated FOXO3 is translocated into the nucleus to further promote muscle degradation what genes? [28] (FIG. 3A).

In addition to cachexia, activin/myostatin signaling has been implicated in malignant angiogenesis regulation in preclinical studies. In a study by Gallot Y S, et al. [14], myostatin knockout mice were found to have significantly lower tumor burden and reduced tumor expression of genes involved in angiogenesis (e.g. vascular endothelial growth factor A [VEGF-A], hypoxia-inducible factor 1α [HIF-1α], etc.), compared to mice with intact myostatin signaling. Furthermore, overexpression of the activin type I receptor, ALK5, has been shown to promote angiogenesis, invasion, and metastatic potential in tumor cells [15]. Conversely, a small molecule inhibitor of the type I activin like receptor (SB431542) has been shown to decrease VEGF expression and inhibit angiogenesis.

Dually phosphorylated Smads upregulate the transcription of c-Myc and MMP-9 [25], which promote angiogenesis in tumor cells (FIG. 2B). Smads with phosphorylated linker-regions also promote the nuclear translocation of FOXO3, which leads to degradation of muscle cells (FIG. 2A).

In this example, the association between genotypes and expression levels of ACVR2B was also evaluated. The expression level of ACVR2B was significantly higher in tumors with C allele compared to those with T allele, indicating that this SNP has a function of expressing ACVR2B mRNA. There was no difference in VEGFA expression among genotypes of ACVR2B.

This example demonstrates associations between genetic variations in cancer cachexia pathways and clinical outcomes of mCRC patients treated with bevacizumab-based chemotherapy. These genetic variants may serve as predictive and/or prognostic biomarkers in mCRC patients treated with anti-angiogenic therapy and inform the future development of drug targeting cancer cachexia.

The disclosure illustratively described herein can suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure claimed.

Thus, it should be understood that although the present disclosure has been specifically disclosed by preferred embodiments and optional features, modification, improvement and variation of the disclosure embodied therein herein disclosed can be resorted to by those skilled in the art, and that such modifications, improvements and variations are considered to be within the scope of this disclosure. The materials, methods, and examples provided here are representative of preferred embodiments, are exemplary, and are not intended as limitations on the scope of the disclosure.

The disclosure has been described broadly and generically herein. Each of the narrower species and subgeneric groupings falling within the generic disclosure also form part of the disclosure. This includes the generic description of the disclosure with a proviso or negative limitation removing any subject matter from the genus, regardless of whether or not the excised material is specifically recited herein.

In addition, where features or aspects of the disclosure are described in terms of Markush groups, those skilled in the art will recognize that the disclosure is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

### REFERENCES

1. Fearon K, Strasser F, Anker S D et al. Definition and classification of cancer cachexia: An international consensus. Lancet Oncol. 2011; 12(5):489-495.
2. Wallengren O, Lundholm K, Bosaeus I. Diagnostic criteria of cancer cachexia: Relation to quality of life, exercise capacity and survival in unselected palliative care patients. Support. Care Cancer 2013; 21(6):1569-1577.
3. Capuano G, Gentile P C, Bianciardi F et al. Prevalence and influence of malnutrition on quality of life and performance status in patients with locally advanced head and neck cancer before treatment. Support. Care Cancer 2010; 18(4):433-437.
4. Morishita S, Kaida K, Tanaka T et al. Prevalence of sarcopenia and relevance of body composition, physiological function, fatigue, and health-related quality of life in patients before allogeneic hematopoietic stem cell transplantation. Support. Care Cancer 2012; 20(12):3161-3168.
5. Miyamoto Y, Baba Y, Sakamoto Y et al. Sarcopenia is a Negative Prognostic Factor After Curative Resection of Colorectal Cancer. Ann. Surg. Oncol. 2015; 22(8):2663-8.
6. Argilés J M, Busquets S, Stemmler B, Lopez-Soriano F J. Cancer cachexia: understanding the molecular basis. Nat. Rev. Cancer 2014; 14(11):754-762.
7. Miyamoto Y, Hanna D L, Zhang W et al. Molecular Pathways: Cachexia Signaling—A Targeted Approach to Cancer Treatment. Clin. cancer Res. 2016; 22(16):3999-4004.
8. Tisdale M J. Reversing cachexia. Cell 2010; 142(4):511-512.
9. Han H Q, Zhou X, Mitch W E, Goldberg A L. Myostatin/activin pathway antagonism: Molecular basis and therapeutic potential. Int. J. Biochem. Cell Biol. 2013; 45(10):2333-2347.
10. Padrao A I, Oliveira P, Vitorino R et al. Bladder cancer-induced skeletal muscle wasting: disclosing the role of mitochondria plasticity. Int. J. Biochem. Cell Biol. 2013; 45(7):1399-1409.
11. Loumaye A, De Barsy M, Nachit M et al. Role of activin A and myostatin in human cancer cachexia. J. Clin. Endocrinol. Metab. 2015; 100(5):2030-2038.
12. Villasenor A, Ballard-Barbash R, Baumgartner K et al. Prevalence and prognostic effect of sarcopenia in breast cancer survivors: the HEAL Study. J. Cancer Surviv. 2012; 6(4):398-406.
13. Jakowlew S B. Transforming growth factor-0 in cancer and metastasis. Cancer Metastasis Rev. 2006; 25(3):435-457.
14. Gallot Y S, Durieux A C, Castells J et al. Myostatin gene inactivation prevents skeletal muscle wasting in cancer. Cancer Res. 2014; 74(24):7344-7356.
15. Safina A, Vandette E, Bakin A V. ALK5 promotes tumor angiogenesis by upregulating matrix metalloproteinase-9 in tumor cells. Oncogene 2007; 26(17):2407-2422.
16. Sorich M J, Wiese M D, Rowland A et al. Extended RAS mutations and anti-EGFR monoclonal antibody survival benefit in metastatic colorectal cancer: a meta-analysis of randomized, controlled trials. Ann. Oncol. 2015; 26(1):13-21.
17. Shiono M, Huang K, Downey R J et al. An analysis of the relationship between metastases and cachexia in lung cancer patients. Cancer Med. 2016:1-8.
18. National Comprehensive Cancer Network. NCCN Guidelines Version 1.2016 Colon Cancer. PubMed Heal. 2016.
19. Garcia-Alfonso P, Grande E, Polo E et al. The role of antiangiogenic agents in the treatment of patients with advanced colorectal cancer according to K-RAS status. Angiogenesis 2014; 17(4):805-821.
20. Heinemann V, Weikersthal L F Von, Decker T et al. FOLFIRI plus cetuximab versus FOLFIRI plus bevacizumab as first-line treatment for patients with metastatic colorectal cancer (FIRE-3): A randomised, open-label, phase 3 trial. Lancet Oncol. 2014; 15(10):1065-1075.
21. Loupakis F, Cremolini C, Masi G et al. Initial Therapy with FOLFOXIRI and Bevacizumab for Metastatic Colorectal Cancer. N. Engl. J. Med. 2014; 371(17):1609-1618.
22. Sunakawa Y, Stintzing S, Cao S et al. Variations in genes regulating tumor-associated macrophages (TAMs) to predict outcomes of bevacizumab-based treatment in patients with metastatic colorectal cancer: Results from TRIBE and FIRE3 trials. Ann. Oncol. 2015; 26(12):2450-2456.
23. McShane L M, Altman D G, Sauerbrei W et al. REporting recommendations for tumor MARKer prognostic studies (REMARK). Nat. Clin. Pract. Oncol. 2005; 2(8):416-422.
24. Lee P H, Shatkay H. F-SNP: computationally predicted functional SNPs for disease association studies. Nucleic Acids Res. 2008; 36(Database issue):D820-4.
25. Matsuzaki K. Smad phosphoisoform signaling specificity: The right place at the right time. Carcinogenesis 2011; 32(11):1578-1588.
26. Cohen S, Nathan J a, Goldberg A L. Muscle wasting in disease: molecular mechanisms and promising therapies. Nat. Rev. Drug Discov. 2015; 14(1):58-74.
27. Bonaldo P, Sandri M. Cellular and molecular mechanisms of muscle atrophy. Dis. Model. Mech. 2013; 6(1):25-39.
28. Frost R a, Lang C H. Protein kinase B/Akt: a nexus of growth factor and cytokine signaling in determining muscle mass. J. Appl. Physiol. 2007; 103(March 2007):378-87.
29. Kretzschmar M, Doody J, Timokhina I, Massague J. A mechanism of repression of TGFbeta/Smad signaling by oncogenic Ras. Genes Dev. 1999; 13(7):804-816.
30. Bakin A V, Tomlinson A K, Bhowmick N A et al. Phosphatidylinositol 3-kinase function is required for transforming growth factor beta-mediated epithelial to mesenchymal transition and cell migration. J. Biol. Chem. 2000; 275(47):36803-36810.
31. Ikushima H, Miyazono K. TGFbeta signalling: a complex web in cancer progression. Nat. Rev. Cancer 2010; 10(6):415-24.
32. Zhou X, Wang J L, Lu J et al. Reversal of cancer cachexia and muscle wasting by ActRIIB antagonism leads to prolonged survival. Cell 2010; 142(4):531-543.
33. Hatakeyama S, Summermatter S, Jourdain M et al. ActRII blockade protects mice from cancer cachexia and prolongs survival in the presence of anti-cancer treatments. Skelet. Muscle 2016; 6(1):26.
34. Jameson G S, Von Hoff D D, Weiss G J et al. Safety of the antimyostatin monoclonal antibody LY2495655 in healthy subjects and patients with advanced cancer. J Clin Oncol 2012; 30:suppl; abstr 2516.

### SEQUENCE LISTING

Region of The following nucleotide sequence represents a region of human DNA comprising, or consisting essentially of, or yet further consisting of the rs1792689 polymorphism in reverse orientation of SMAD2 gene:

(SEQ ID NO: 1)
TATCTACATTCTCTCTCAGGTGTTC[C/T]ATTTTGGATGATGGTGAATA
ATAAG

A region of human DNA comprising, or consisting essentially of, or yet further consisting of the rs2268753 polymorphism in the ACVR2B gene:

(SEQ ID NO: 2)
GTATCGGTTCAGGAGTTTAGATCCA[C/T]TCACGGATACTGACCTGTCA
CCATG

The following nucleotide sequence represents a region of human DNA comprising, or consisting essentially of or yet further consisting of the rs17776182 polymorphism located within the INHBA antisense RNA 1 (INHBA-AS1) gene:

(SEQ ID NO: 3)
TGTTTTTAGATGAAGGTGGAAATAC[A/G]ATGAAGATGATGCTCTGTTA
GTTAT
rs1792689 forward primer:
(SEQ ID NO: 4)
GCCAGGATGGTCTCAATCTC
rs1792689 reverse primer:
(SEQ ID NO: 5)
TGATCTTATTATTCACCATCATCCA
rs2268753 forward primer:
(SEQ ID NO: 6)
GGAGCTCAGGGTAGTGCAAA
rs2268753 reverse primer:
(SEQ ID NO: 7)
GGACCCTGCCTCAGGACTAT
rs17776182 forward primer:
(SEQ ID NO: 8)
TGTGATAGCCACAGCCTCAA
rs17776182 reverse primer:
(SEQ ID NO: 9)
TTCCAAACCTCAGTGGCTTC

The amplicon generated comprising, or consisting essentially of, or yet further consisting of SMAD2 rs1792689 has the following sequence (Y=C or T):

(SEQ ID NO: 10)
5′GCCAGGATGGTCTCAATCTCTTGACCTTGTGATCCGCCTGCCTCGGCC
TCCCAAAGTGCTGGGCTTACAGGTGTGAGCCACCACGCCTGGCCCTGGCC
TGATATTAATAGTATCTACATTCTCTCTCAGGTGTTCYATTTTGGATGAT
GGTGAATAATAAGATCA 3′

The amplicon generated comprising, or consisting essentially of, or yet further consisting of ACVR2B rs2268753 has the following sequence (Y=C or T):

(SEQ ID NO: 11)
5′GGGAGCTCAGGGTAGTGCAAATGAGAACCAAGGAGTATCGGTTCAGGA
GTTTAGATCCAYTCACGGATACTGACCTGTCACCATGGATTGGGATCTGG
AGGGTTGAGGACTGGGTCTGGATAATATTTTTGCTAGTGACTGTAGATAG
ACTCTAGATAGTCCTGAGGCAGGGTCC3′

The amplicon generated comprising, or consisting essentially of, or yet further consisting of INHBA rs17776182 has the following sequence (R=A or G):

(SEQ ID NO: 12)
5′TGTGATAGCCACAGCCTCAAGGCTGTTTTTAGATGAAGGTGGAAATAC
RATGAAGATGATGCTCTGTTAGTTATCATTGATCAAGCACTCATTTCTGC
TACGCATGGAGCAAAGTGCTTGATATATGTGTAATACCCTTTCACCCTTC
AAATAACAATTTGAAATAGGTTTTCTTATTATTGCTAATTACAGATGAAG
CCACTGAGGTTTGGAA3′
rs7570532 forward primer:
(SEQ ID NO: 13)
CATCAGCGGATGAATGGATA
rs7570532 reverse primer:
(SEQ ID NO: 14)
GCATAGCTTAGCTCGCACTTG
rs4946935 forward primer:
(SEQ ID NO: 15)
CTCAGTCCGGAAGTCTAGAACAG
rs4946935 reverse primer:
(SEQ ID NO: 16)
AAAATGCTCTGAAGTTGAAAAGC

A region of human DNA comprising, or consisting essentially of, or yet further consisting of the rs7570532 polymorphism within the MSTN gene:

(SEQ ID NO: 17)
ATACTATTTAACCATAAAAAAGAGT[A/G]AAGGAATGTC
TTTTGCAGCAAATTA

A region of human DNA comprising, or consisting essentially of, or yet further consisting of the rs4946935 polymorphism within the FOXO3 gene:

(SEQ ID NO: 18)
AAGGACCCACCAAAA CACCCCTAAT[A/G]TGGCTTTCTT
TATCTCCCAA

What is claimed is:

1. A method for treating a colorectal cancer patient with a therapy comprising an effective amount of irinotecan and bevacizumab, wherein a sample isolated from the patient is characterized by a polymorphism of the group of (G/G) for rs1792689, (C/T) or (C/C) for rs2268753, (G/G) for rs17776182, (A/A) for rs7570532, and (A/G) or (G/G) for rs4946935.

2. The method of claim 1, wherein the colorectal cancer patient is suffering from metastatic colorectal cancer.

3. The method of claim 1, wherein the therapy further comprises a therapeutically effective amount of folinic acid and/or a pyrimidine analog.

4. The method of claim 3, wherein the therapy further comprises a therapeutically effective amount of leucovorin and/or fluorouracil (5-FU).

5. The method of claim 1, wherein the therapy is a first-line therapy.

6. The method of claim 1, wherein the therapy is subsequent to the first line therapy.

7. A method for treating a colorectal cancer patient with a therapy excluding an effective amount of irinotecan and bevacizumab, wherein a sample isolated from the patient is characterized by a polymorphism of the group of (A/G) or (A/A) for rs1792689, (T/T) for rs2268753, (A/G) or (A/A) for rs17776182, (A/G) for rs7570532, and (A/A) for rs4946935.

8. The method of claim 7, wherein the colorectal cancer patient is suffering from metastatic colorectal cancer.

9. The method of claim 7, wherein the therapy further comprises a therapeutically effective amount of folinic acid and/or a pyrimidine analog.

10. The method of claim 9, wherein the therapy further comprises a therapeutically effective amount of leucovorin and/or fluorouracil (5-FU).

11. The method of claim 7, wherein the therapy is a first-line therapy.

12. The method of claim 7, wherein the therapy is subsequent to the first line therapy.

13. A method for treating a colorectal cancer patient with an effective amount of a therapy comprising irinotecan and bevacizumab, the method comprising determining if the patient's sample comprises a polymorphism from the group of rs1792689, rs2268753, rs17776182, rs7570532 and rs4946935; and if the patient has (G/G) for rs1792689, (C/T) or (C/C) for rs2268753, (G/G) for rs17776182, (A/A) for rs7570532, and (A/G) or (G/G) for rs4946935, then administering an effective amount of the therapy.

14. The method of claim 13, wherein the colorectal cancer patient is suffering from metastatic colorectal cancer.

15. The method of claim 13, wherein the therapy further comprises a therapeutically effective amount of folinic acid and/or a pyrimidine analog.

16. The method of claim 15, wherein the therapy further comprises a therapeutically effective amount of leucovorin and/or fluorouracil (5-FU).

17. The method of claim 13, wherein the therapy is a first-line therapy.

18. The method of claim 13, wherein the therapy is subsequent to the first line therapy.


SEQUENCE LISTING (34 sequences)

SEQ ID NO: 1 - DNA, 51 bp, Homo sapiens
tatctacattctctctcaggtgttcyattttggatgatggtgaataataag

SEQ ID NO: 2 - DNA, 51 bp, Homo sapiens
gtatcggttcaggagtttagatccaytcacggatactgacctgtcaccatg

SEQ ID NO: 3 - DNA, 51 bp, Homo sapiens
tgtttttagatgaaggtggaaatacratgaagatgatgctctgttagttat

SEQ ID NO: 4 - DNA, 20 bp, Artificial Sequence
  Description of Artificial Sequence Synthetic primer
gccaggatggtctcaatctc

SEQ ID NO: 5 - DNA, 25 bp, Artificial Sequence
  Description of Artificial Sequence Synthetic primer
tgatcttattattcaccatcatcca

SEQ ID NO: 6 - DNA, 20 bp, Artificial Sequence
  Description of Artificial Sequence Synthetic primer
ggagctcagggtagtgcaaa

SEQ ID NO: 7 - DNA, 20 bp, Artificial Sequence
  Description of Artificial Sequence Synthetic primer
ggaccctgcctcaggactat

SEQ ID NO: 8 - DNA, 20 bp, Artificial Sequence
  Description of Artificial Sequence Synthetic primer
tgtgatagccacagcctcaa

SEQ ID NO: 9 - DNA, 20 bp, Artificial Sequence
  Description of Artificial Sequence Synthetic primer
ttccaaacctcagtggcttc

SEQ ID NO: 10 - DNA, 165 bp, Artificial Sequence
  Description of Artificial Sequence Synthetic polynucleotide
gccaggatggtctcaatctcttgaccttgtgatccgcctgcctcggcctcccaaagtgctgggcttacaggtgtgagccaccacgcctggccctggcctgatattaatagtatctacattctctctcaggtgttcyattttggatgatggtgaataataagatca

SEQ ID NO: 11 - DNA, 175 bp, Artificial Sequence
  Description of Artificial Sequence Synthetic polynucleotide
gggagctcagggtagtgcaaatgagaaccaaggagtatcggttcaggagtttagatccaytcacggatactgacctgtcaccatggattgggatctggagggttgaggactgggtctggataatatttttgctagtgactgtagatagactctagatagtcctgaggcagggtcc

SEQ ID NO: 12 - DNA, 214 bp, Artificial Sequence
  Description of Artificial Sequence Synthetic polynucleotide
tgtgatagccacagcctcaaggctgtttttagatgaaggtggaaatacratgaagatgatgctctgttagttatcattgatcaagcactcatttctgctacgcatggagcaaagtgcttgatatatgtgtaataccctttcacccttcaaataacaatttgaaataggttttcttattattgctaattacagatgaagccactgaggtttggaa

SEQ ID NO: 13 - DNA, 20 bp, Artificial Sequence
  Description of Artificial Sequence Synthetic primer
catcagcggatgaatggata

SEQ ID NO: 14 - DNA, 21 bp, Artificial Sequence
  Description of Artificial Sequence Synthetic primer
gcatagcttagctcgcacttg

SEQ ID NO: 15 - DNA, 23 bp, Artificial Sequence
  Description of Artificial Sequence Synthetic primer
ctcagtccggaagtctagaacag

SEQ ID NO: 16 - DNA, 23 bp, Artificial Sequence
  Description of Artificial Sequence Synthetic primer
aaaatgctctgaagttgaaaagc

SEQ ID NO: 17 - DNA, 51 bp, Homo sapiens
atactatttaaccataaaaaagagtraaggaatgtcttttgcagcaaatta

SEQ ID NO: 18 - DNA, 46 bp, Homo sapiens
aaggacccaccaaaacacccctaatrtggctttctttatctcccaa

SEQ ID NO: 19 - DNA, 25 bp, Artificial Sequence
  Description of Artificial Sequence Synthetic primer
aagatttcactgtgcctagatatgg

SEQ ID NO: 20 - DNA, 25 bp, Artificial Sequence
  Description of Artificial Sequence Synthetic primer
aaggaggaataaccttaagacctca

SEQ ID NO: 21 - DNA, 20 bp, Artificial Sequence
  Description of Artificial Sequence Synthetic primer
ctggtggaagtcttgggtgt

SEQ ID NO: 22 - DNA, 22 bp, Artificial Sequence
  Description of Artificial Sequence Synthetic primer
tctgggagatgaaagacagatg

SEQ ID NO: 23 - DNA, 20 bp, Artificial Sequence
  Description of Artificial Sequence Synthetic primer
cttgccttaccatgggagaa

SEQ ID NO: 24 - DNA, 20 bp, Artificial Sequence
  Description of Artificial Sequence Synthetic primer
atttcctcagggacacacca

SEQ ID NO: 25 - DNA, 21 bp, Artificial Sequence
  Description of Artificial Sequence Synthetic primer
ccagagctcaccatgtatcct

SEQ ID NO: 26 - DNA, 20 bp, Artificial Sequence
  Description of Artificial Sequence Synthetic primer
ttatgatgtgaccgcctctg

SEQ ID NO: 27 - DNA, 20 bp, Artificial Sequence
  Description of Artificial Sequence Synthetic primer
aagactgccagtgagggaag

SEQ ID NO: 28 - DNA, 20 bp, Artificial Sequence
  Description of Artificial Sequence Synthetic primer
ggcattgttgtggatttgtg

SEQ ID NO: 29 - DNA, 22 bp, Artificial Sequence
  Description of Artificial Sequence Synthetic primer
tggacacgattattccgcaaaa

SEQ ID NO: 30 - DNA, 19 bp, Artificial Sequence
  Description of Artificial Sequence Synthetic primer
tgaccttgtgatccgcctg

SEQ ID NO: 31 - DNA, 20 bp, Artificial Sequence
  Description of Artificial Sequence Synthetic primer
gccacagagaaaggaaaaca

SEQ ID NO: 32 - DNA, 20 bp, Artificial Sequence
  Description of Artificial Sequence Synthetic primer
taccctccaaacagttaaca

SEQ ID NO: 33 - DNA, 20 bp, Artificial Sequence
  Description of Artificial Sequence Synthetic primer
tcagttgggttggaattggt

SEQ ID NO: 34 - DNA, 20 bp, Artificial Sequence
  Description of Artificial Sequence Synthetic primer
ccctctgcgttagattctgg